(19) United States
(12) Patent Application Publication
(10) Pub. No.: US 2013/0251731 A1
(43) Pub. Date: Sep. 26, 2013

(54) Tau Acetylation in the Pathogenesis of Alzheimers Disease and Other Related Tauopathies

(71) Applicant: THE TRUSTEES OF THE UNIVERSITY OF PENNSYLVANIA, Philadelphia, PA (US)

(72) Inventors: Virginia M.Y. Lee, Philadelphia, PA (US); Todd J. Cohen, Philadelphia, PA (US); John Q. Trojanowski, Philadelphia, PA (US)

(73) Assignee: The Trustees of the University of Pennsylvania, Philadelphia, PA (US)

(21) Appl. No.: 13/799,842

(22) Filed: Mar. 13, 2013

Related U.S. Application Data
(60) Provisional application No. 61/614,323, filed on Mar. 22, 2012.

(51) Int. Cl. C12Q 1/48

(52) U.S. Cl. CPC C12Q 1/48
USPC 424/158.1; 435/7.5; 435/7.1; 435/7.4; 530/389.1; 536/23.1; 514/17.7; 514/44 R

(57) ABSTRACT

The present invention relates to biomarkers and diagnostic methods for Alzheimer's disease and other neurodegenerative disorders. The invention also provides compositions for detecting the biomarker as well as compositions and methods useful for treating Alzheimer's disease and other neurodegenerative disorders.

### CROSS-REFERENCES TO RELATED APPLICATIONS

This application claims priority under 35 U.S.C. §119(e)(1) to U.S. Provisional Application No. 61/614,323, filed Mar. 22, 2012, which is incorporated herein by reference in its entirety.

### STATEMENT REGARDING FEDERALLY SPONSORED RESEARCH OR DEVELOPMENT

This invention was made with government support under AG17586 and AG010124 awarded by the National Institutes of Health (NIH). The government has certain rights in the invention.

### BACKGROUND OF THE INVENTION

Tau proteins are expressed primarily in the central nervous system where the critical functions of promoting microtubule (MT) assembly and stability are mediated by six tau isoforms generated by mRNA alternative splicing containing either three (3R-tau) or four (4R-tau) MT-binding repeats (Andreadis, et al., 1992, Biochemistry 31:10626-10633; Goedert, et al., 1989, Neuron 3:519-526). Regulation of tau binding to MTs is mediated by serine/threonine phosphorylation, which decreases tau—MT-binding affinity (Biernat, et al., 1993, Neuron 11:153-163; Drechsel, et al., 1992, Mol. Biol. Cell 3:1141-1154; Bramblett, et al., 1993, Neuron 10:1089-1099). However, aberrant accumulation of unbound hyperphosphorylated insoluble tau as neurofibrillary tangles (NFTs) is implicated in the pathogenesis of neurodegenerative tauopathies including Alzheimer's disease (AD; for review see Lee, et al., 2001, Annu Rev. Neurosci. 24:1121-1159; and Goedert & Jakes, 2005, Biochim. Biophys. Acta 1739:240-250). Genetic studies demonstrated that missense mutations that alter tau binding and MT assembly (for example, ΔK280, V337M, P301L, R406W) are pathogenic for frontotemporal dementia and parkinsonism linked to chromosome 17 (FTDP-17), which is characterized by prominent NFTs and neurodegeneration (Hong, et al., 1998, Science 282:1914-1917; Hutton et al., 1998, Nature 393, 702-705; Rizzu, et al., 1999, Am. J. Hum. Genet. 64:414-421; Poorkaj, et al., 1998, Ann. Neurol. 43:815-825).

The mechanism that causes normal soluble tau to become hyperphosphorylated and disengaged from MTs to form tau inclusions remains unknown and post-translational modifications other than phosphorylation could regulate tau function and aggregation. Notably, reversible lysine acetylation has emerged as a potential regulatory modification implicated in AD and other neurodegenerative disorders, and a recent study demonstrated that tau is modified by lysine acetylation (Min, et al., 2010, Neuron 67:953-966). However, the functional significance and implications of tau acetylation in neurodegenerative tauopathies are not clear.

The identification of those afflicted, or those at risk of becoming afflicted, with AD or other neurodegenerative disease remains an elusive goal in the field. The discovery of biomarkers that can identify and/or discriminate neurodegenerative diseases is therefore critical for the diagnosis and prognosis of a patient. There is a need in the art for methods of diagnosing a neurodegenerative disorder. The present invention addresses this unmet need.

### SUMMARY OF THE INVENTION

The invention relates to a method of diagnosing a neurodegenerative disorder in a subject. The method comprises determining the level of at least one biomarker in a biological sample obtained from the subject, wherein an elevated level of the biomarker in the sample compared to the level of the biomarker in a control sample is an indication of a neurodegenerative disorder, and further wherein the at least one biomarker comprises acetylated tau. In one embodiment, the neurodegenerative disorder is at least one disorder selected from the group consisting of Alzheimer's Disease (AD), corticobasal degeneration (CBD), Pick's disease (PiD), progressive supranuclear palsy, argyrophilic grain disease, tangle predominant senile dementia, Guam parkinsonism-dementia complex, frontotemporal dementia, frontotemporal lobar degeneration, FTDP-17, Lytico-Bodig disease, and Parkinson's disease. In another embodiment, acetylated tau is acetylated on at least one lysine residue selected from the group consisting of K150, K163, K174, K234, K240, K259, K274, K280, K281, K290, K311, K369, and K395. In yet another embodiment, the biomarker is detected by a method selected from the group consisting of immunohistochemistry, immunocytochemistry, immunofluorescence, immunoprecipitation, western blotting, and ELISA.

The invention also includes a method of diagnosing a neurodegenerative disorder in a subject. The method comprises determining the level of at least one biomarker in a biological sample obtained from the subject, wherein the biomarker differentially discriminates between neurodegenerative disorders, and wherein the biomarker is acetylated tau. In one embodiment, the biomarker differentially discriminates between neurodegenerative disorders associated with 4R-tau and neurodegenerative disorders not associated with 4R-tau. In another embodiment, acetylated tau is acetylated on at least one lysine residue selected from the group consisting of K150, K163, K174, K234, K240, K259, K274, K280, K281, K290, K311, K369, and K395. In yet another embodiment, the biomarker is detected by at least one method selected from the group consisting of immunohistochemistry, immunofluorescence, western blotting, and ELISA.

Also included in the invention is a composition that specifically recognizes a biomarker associated with a neurodegenerative disorder, wherein the biomarker is acetylated tau. In one embodiment, acetylated tau is acetylated on at least one lysine residue selected from the group consisting of K150, K163, K174, K234, K240, K259, K274, K280, K281, K290, K311, K369, and K395. In embodiment, the neurodegenerative disorder Alzheimer's Disease (AD), corticobasal degeneration (CBD), Pick's disease (PiD), progressive supranuclear palsy, argyrophilic grain disease, tangle predominant senile dementia, Guam parkinsonism-dementia complex, frontotemporal dementia, frontotemporal lobar degeneration, FTDP-17, Lytico-Bodig disease, and Parkinson's disease. In one embodiment, the composition comprises an antibody or fragment thereof.

The invention also includes a composition for treating a neurodegenerative disorder in a subject, wherein the composition reduces the level of tau acetylation. In one embodiment, the composition enhances the activity of at least one deacetylase. In another embodiment, the composition reduces the activity of at least one acetyltransferase. In yet another embodiment, the composition is selected from the group consisting of a peptide, a nucleic acid, an antibody, and a small molecule.

Further included in the invention is a method of treating a neurodegenerative disorder in a subject. The method comprises administering to the subject an effective amount of a composition, wherein the composition reduces the level of tau acetylation. In one embodiment, the composition enhances the activity of at least one deacetylase. In another embodiment, the composition reduces the activity of at least one acetyl-transferase. In yet another embodiment, the composition is selected from the group consisting of a peptide, a nucleic acid, an antibody, and a small molecule.

### BRIEF DESCRIPTION OF THE DRAWINGS

The following detailed description of preferred embodiments of the invention will be better understood when read in conjunction with the appended drawings. For the purpose of illustrating the invention, there are shown in the drawings embodiments which are presently preferred. It should be understood, however, that the invention is not limited to the precise arrangements and instrumentalities of the embodiments shown in the drawings.

FIG. 1, comprising FIG. 1A through FIG. 1F, depicts the results of experiments which demonstrate that tau acetylation impairs microtubule assembly and promotes tau fibrillization in vitro. Recombinant full-length T40, 4R-tau MT-binding domain K18, or tau fibrils were acetylated by incubation with [¹⁴C]-labeled acetyl-CoA in the presence or absence of CBP. FIG. 1A illustrates that the reaction products were analyzed by SDS-PAGE and Coomassie blue staining followed by overnight radiographic exposure using STORM phosphor-imager software. FIG. 1B depicts the results of an experiment where T40 or K18 proteins were incubated with acetyl-CoA and/or recombinant CBP and reaction products were subjected to immunoblot analysis using a polyclonal anti-tau-specific antibody for MT repeat domain (E10) and an anti-acetyl-lysine antibody. FIG. 1C is a graph depicting the MT assembly activities of K18 proteins as evaluated in light-scattering assays. Tubulin monomers (30 μM) were mixed with 40 μM tau proteins in MT assembly buffer supplemented with 2 mM guanosine triphosphate. MT assembly was determined by monitoring absorbance every minute at 350 nm using a SpectraMax plate reader. Colour key is as follows: native unmodified K18 (pink squares), acetylated K18 (blue triangles), K18 containing K311D mutation (turquoise X's) and no tau control (black diamonds). FIG. 1D is a graph illustrating the results of experiments where Tau proteins (10 μM) were evaluated in fibrillization reactions using 10 μM heparin to induce assembly. At each time point, samples were incubated with 12.5 μM ThT and excitation/emission wavelengths were set to 450 and 510 nm, respectively. Legend key is as follows: native unmodified K18 (squares), acetylated K18 (diamonds) and K18-containing K311D mutation (triangles). Both MT assembly and fibrillization reactions were confirmed from n=4 independent experiments. Error bars indicate standard error of the mean. FIG. 1E depicts the results of experiments where Tau proteins at indicated fibrillization time points (T=0, 1, 3 h) were centrifuged at 100,000 g for 30 min to generate a pellet fraction (P) containing tau fibrils and supernatants (S) containing unassembled tau protein. Samples were analysed by SDS-PAGE and Coomassie staining to monitor fibril formation. FIG. 1F illustrates the results of experiments where four-hour time points from fibrillization reactions were analysed by negative-staining EM. Note, at the concentration of 10 μM, native K18 did not fibrillize, whereas acetylated K18 had fibrillized extensively with morphologies similar to AD-like PHFs. Scale bar, 200 nm.

FIG. 2, comprising FIG. 2A through FIG. 2K, depicts the results of experiments which illustrate that tau acetylation in cells inhibits tau-mediated MT stabilization. FIG. 2A depicts the results of an experiment where Doxycycline (Dox)-treated HEK-T40 cells were cultured in the presence of either trichostatin A (TSA), nicotinamide (NCA) or both and pulse labelled with [³H]-acetate for 2 h. Tau was immunoprecipitated, treated with λ-phosphatase where indicated, and analyzed by SDS-PAGE followed by autoradiography and exposure to film for 2 weeks. Total cellular lysates were analyzed by immunoblotting using anti-tau (T14/T46), PHF-1 and GAPDH antibodies. FIG. 2B illustrates the results of an experiment where cells transfected with CBP were treated with TSA and acetylated tau levels were determined by immunoprecipitation/immunoblot analysis using anti-acetylated lysine antibody. Cellular lysates were immunoblotted using T14/46, PHF-1, AT8 and GAPDH antibodies. FIG. 2C depicts the results of an experiment where cells were transfected with CBP and either wild-type (WT) HDAC6 or the catalytically dead (CD) HDAC6-H611A mutant and tau acetylation was determined by immunoblotting similar to FIG. 2B above. FIG. 2D depicts the results of an experiment where QBI-293 cells were transfected with WT T40, 2KR (K280/281R), 3KR (K163/280/281R) or 4KR (K163/280/281/369R) mutant T40 and cell lysates were immunoblotted using anti-acetyl-lysine and total tau (T14/T46) antibodies. FIG. 2E through FIG. 2G are images illustrating the results of an experiment where QBI-293 cells were transfected with WT T40 (FIG. 2E) and total MT bundling was determined by immunofluorescence using anti-acetylated tubulin antibodies as shown for WT (FIG. 2F). Merged images indicate stabilized microtubules as shown for WT (FIG. 2G). FIG. 2H through FIG. 2J are images illustrating the results of an experiment where QBI-293 cells were transfected with 4KQ (K163/280/281/369Q) (FIG. 2H) and MT bundling was determined by immunofluorescence using anti-acetylated tubulin antibodies as shown for 4KQ (FIG. 2I) and merged images (FIG. 2J). Representative images are shown from n=4 independent experiments. Note, WT tau (FIG. 2E-FIG. 2G), but not 4KQ (FIG. 2H-FIG. 2J), promotes robust MT bundling. Scale bar, 50 nm. FIG. 2K is a graph illustrating the results of an experiment where QBI-293 cells were transfected with WT T40, K280Q, 4KQ (K163/280/281/369Q) or 4KR mutant plasmids and MT bundling was determined from tau-expressing cells by quantification of ten fields/transfection and values are represented as % MT bundling from tau-expressing cells. Error bars indicate standard error of the mean. *P value=0.006, ***P value=8.46×10⁻⁶ as determined by Student's t-test.

FIG. 3, comprising FIG. 3A through FIG. 3N, depicts results of experiments which demonstrate tau acetylation in Tg mouse models with tau pathology. (a-f) FIG. 3A through FIG. 3F are images where immunohistochemistry (IHC) was used to analyze cortical sections from the following lines of mice: wild-type (WT) mice using anti-ac-K280 (FIG. 3A) or AT8 (FIG. 3B) antibodies, PS19 monogenic mice using anti-ac-K280 (FIG. 3C) or AT8 antibodies (FIG. 3D) and PS19; PDAPP bigenic mice using anti-ac-K280 (FIG. 3E) and AT8 antibodies (FIG. 3F). FIG. 3G through FIG. 3I are images illustrating the results of an experiment where double-labeling immunofluorescence microscopy using anti-ac-K280 (FIG. 3G) and AT8 (FIG. 3H) antibodies on hippocampal sections from PS19; PDAPP mice showed extensive co-localization of acetylated and hyperphosphorylated tau inclusions (merged image, FIG. 3I). FIG. 3J through FIG. 3L are images illustrating the results of an experiment where double labeling using anti-ac-K280 (FIG. 3J) and ThS (FIG. 3K), an amyloid-binding dye, also demonstrated co-localization of acetylated tau with Thioflavin-S-positive NFTs (merged image, FIG. 3L). Scale bars, 100 μm. FIG. 3M and FIG. 3N depict the results of an experiment where sequential biochemical fractionation was performed on WT, PS19 and PS19/PDAPP mouse cortices using RIPA buffer followed by urea extraction. Soluble (FIG. 3M) and insoluble (FIG. 3N) extracted tau proteins were analyzed by western blotting using anti-ac-K280, PHF-1 and total tau (T14/T46) antibodies.

FIG. 4, comprising FIG. 4A through FIG. 4P, depicts the results of an experiment which illustrates that tau acetylation is associated with tau aggregation in human tauopathies. FIG. 4A through FIG. 4F are images depicting the results of an experiment where immunohistochemistry (IHC) was used to analyze cortical sections from the following tauopathies: Alzheimer's disease (AD) using anti-ac-K280 (FIG. 4A) or PHF-1 (FIG. 4B) antibodies, corticobasal degeneration (CBD) using anti-ac-K280 (FIG. 4C) or PHF-1 (FIG. 4D) antibodies and Pick's disease (PiD) using anti-ac-K280 (FIG. 4E) or PHF-1 (FIG. 4F) antibodies. Shown are representative images of tau inclusions with anti-ac-K280 and PHF-1 immunoreactivity. AD and 4R-tau predominant tauopathies (for example, corticobasal degeneration) were ac-K280 immuno-positive (FIG. 4A and FIG. 4C), whereas 3R-tau predominant Pick's disease was negative (FIG. 4E). FIG. 4G through FIG. 4I are images depicting the results of an experiment where double-labeling immunofluorescence was performed on cortical sections from AD using anti-ac-K280 (FIG. 4G) and PHF-1 (FIG. 4H) antibodies (merged image, FIG. 4I). FIG. 4J through FIG. 4L are images depicting the results of an experiment using a similar analysis performed using sections from CBD using anti-ac-K280 (FIG. 4J) and PHF-1 (FIG. 4K) antibodies (merged image, FIG. 4L). FIG. 4M through FIG. 4O are images depicting the results of an experiment where double labeling of neurofibrillary tangles (NFTs) in AD cortex was detected using anti-ac-K280 (FIG. 4M) and Thioflavin S (FIG. 4N) and the corresponding merged image is shown in FIG. 4O. Insets represent ×60 magnification. Scale bar, 50 μm. FIG. 4P depicts the results of an experiment where biochemical isolation of enriched PHFs was performed on frontal cortex from indicated tauopathies and solubilized PHF-tau was analysed by immunoblotting using anti-ac-K280, PHF-1 and total tau (T14/T46) antibodies. Ac-K280 and PHF-1 immunoreactivity was quantified using Multi Gauge v2.3 and raw intensities are represented below as percent modified tau species/total tau intensities to compare acetylated versus phosphorylated tau ratios.

FIG. 5, comprising FIG. 5A and FIG. 5B, depicts the results of experiments illustrating that Tau T40 acetylation impairs MT assembly and promotes PHF aggregation in vitro. FIG. 5A is a graph illustrating MT assembly activities of either unmodified or acetylated T40 proteins as evaluated in light scattering assays similar to that shown in FIG. 1. 30 μM tubulin monomers were mixed with 15 μM tau proteins in MT assembly (RA) buffer supplemented with 2 mM guanosine triphosphate (GTP). MT assembly was determined by monitoring absorbance every minute at 350 nm using a SpectraMax plate reader. FIG. 5B depicts a graph illustrating the results of an experiment where 15 μM tau proteins were evaluated in fibrillization reactions using 10 μM heparin to induce assembly for 3 days, at which point samples were incubated with 12.5 μM ThT. These results were confirmed from n=4 independent experiments. Error bars indicate standard error of the mean (SEM).

FIG. 6, comprising FIG. 6A through FIG. 6C, depicts the results of experiments which illustrate that mass spectrometry analysis identified K280 as a tau acetylation site in HEK-T40 cells. FIG. 6A illustrates the results of an experiment where HEK-T40 cells treated with Dox were transfected with vector alone or CBP and T40 was immunoprecipitated with T14/T46 antibodies and was separated by SDS-PAGE followed by gel excision and mass spectrometry analysis. In the absence of CBP, no acetylation was detected. FIG. 6B illustrates that in the presence of CBP, acetylation was detected on the four indicated lysine residues with significant ion scores and p-values. Acetylated K280 was detected on the peptide sequence VQIINKK (SEQ ID NO. 3). FIG. 6C illustrates the corresponding m/z spectrum. Ion scores are represented in FIG. 10.

FIG. 7, comprising FIG. 7A through FIG. 7D, depicts the results of experiments which illustrate the characterization of the ac-K280 site-specific acetylated tau antibody. FIG. 7A illustrates the results of an experiment where recombinant WT-K18 or ΔK280-K18 proteins (1 μg) were incubated in the presence or absence of acetyl-CoA or CBP as indicated and K280 acetylation was determined by immunoblotting using anti-ac-K280 and a polyclonal antibody raised to exon 10 of tau protein (E10). FIG. 7B illustrates the results of an experiment where QBI-293 cells were co-transfected with WT-T40 tau or ΔK280-T40 mutant tau expression plasmids along with CBP to promote tau acetylation. 1, 2, and 5 μg total cell lysates were analyzed by western blotting using anti-ac-K280, total tau (T46), and GAPDH antibodies. FIG. 7C is a series of images illustrating the results of an experiment where QBI-293 cells were transfected with WT-40, WT-CBP-HA, or enyzyme-inactive CBP-LD-HA expression plasmids and analyzed by immunofluroescence using anti-tau (T46), monoclonal HA (Santa Cruz), or anti-ac-K280 antibodies. Note, ac-K280 reactivity is specifically detected in the presence of WT-CBP, but not the inactive mutant CBP-LD. Scale bar represents 50 μm. FIG. 7D illustrates the results of an experiment where control or HDAC6 siRNA expressing HEK-T40 cells were analyzed by immunoblotting using ac-K280, total tau, HDAC6, and GAPDH antibodies.

FIG. 8, comprising FIG. 8A through FIG. 8C, depicts the results of experiments illustrating that Ac-K280 pathology accumulates with age in PS19/PDAPP mice. FIG. 8A through FIG. 8C are images depicting the results of an experiment where cortical brain sections from PS19/PDAPP mice at 4-month (FIG. 8A), 8-month (FIG. 8B), or 11-months (FIG. 8C) were analyzed by IHC using anti-ac-K280 antibody. Shown are representative images highlighting increased ac-K280 immuno-reactivity in older mice. Scale bar represents 100 μM.

FIG. 9, comprising FIG. 9A through FIG. 9H, depicts the results of experiments illustrating that Ac-K280-positive tau inclusions are detected in a diverse set of human tauopathies. FIG. 9A through FIG. 9H are images depicting the results of an experiment where cortical brain sections from the indicated tauopathies were analyzed by IHC using anti-ac-K280 (FIG. 9A through FIG. 9D) and PHF-1 (FIG. 9E through FIG. 9H) antibodies. Shown are representative images highlighting NFTs and tau inclusions in each tauopathy. Scale bar represents 50 μM, insets depict 60× magnification.

FIG. 10 depicts the ion scores from acetylated VQIINNK peptide on residue Lys-280 identified by mass spectrometry analysis of tau in cells. Tau-T40 was immunoprecipitated with T14/T46 antibodies and separated by SDS-PAGE followed by gel excision and mass spectrometry analysis. Listed are the ion scores that correspond to the m/z spectrum depicted in FIG. 6. The numbers in red bold are statistically significant and confirm the presence of acetylation on Lys-280 (K280). These data were generated using Mascot software (Matrix Science).

FIG. 11, comprising FIGS. 11a-11e, depicts the results of a series of experiments showing that Tau proteins containing MT-binding repeats possess robust acetyltransferase activity. FIG. 11a is a schematic representation of the purified tau proteins used the present invention: tau proteins with 3 or 4 MT-binding repeats (3R- or 4R-tau) and a variable number of N-terminal inserts (0-2N), and tau lacking all MT-binding repeats but containing both N-terminal inserts (no repeat tau). Location of C291A and C322A mutations in 3R- and 4R-tau are highlighted. FIG. 11b is an image of an SDS-PAGE and Coomassie blue (top) gel of tau proteins incubated with [¹⁴C]-labeled acetyl-CoA followed by autoradiography (bottom) to detect acetylated tau proteins. FIG. 11c shows the same acetyltransferase assay as in FIG. 11b, with 3R-tau or 4R-tau proteins. FIG. 11d is an image of an immunoblot of 3R or 4R-tau isoforms incubated in the absence or presence of cold CoA or acetyl-CoA, using acetyl-lysine, Ac-K280, and total tau (T46) antibodies. FIG. 11e depicts the results of a gel acetyltransferase assay with Tau-K18, 0N4R-tau, 2N4R-tau, and tau-K18(−), followed by Commassie staining (top) and autoradiography (bottom).

FIG. 12, comprising FIGS. 12a and 12b, depicts the results of experiments showing that mouse and human tau proteins possess acetyltransferase activity. FIG. 12a is an image of an immunoblot analysis of heat stable brain extracts from wild-type (WT) and tau knock-out (KO) mice incubated with CoA or acetyl-CoA, using anti-acetyl-lysine, Ac-K280, total tau (T49), and GAPDH antibodies. Unboiled brain extracts were used to normalize protein loading using GAPDH immunoblotting (bottom panel). FIG. 12b is an image of an immunoblot analysis of high-salt extracted tau proteins from control and Alzheimer brain similar to FIG. 12a.

FIG. 13, comprising FIGS. 13a-13g, depicts the results of experiments showing that cysteine residues are required for tau-mediated acetyltransferase activity. FIG. 13a is the amino acid sequence alignment of a minimal region of tau repeats 2 and 3 with MYST family acetyltransferases ESA1 and Tip60; identical or conserved amino acid residues are in red. Putative tau catalytic cysteine residues are marked by the blue triangle. Disease-associated tau mutations (L315R, K317M, and S320F) are represented by blue circles. FIG. 13b is an image showing cell-based acetyltransferase assays using [¹⁴C]-labeled acetyl-CoA that identifies acetylated tau proteins visualized by Coomassie staining (top) and autoradiography (bottom). Deletions of individual repeat regions correspond to the following amino acid residues in full-length 2N4R tau: ΔR1: 244-274, ΔR2: 275-305, ΔR3: 306-336, ΔR4: 337-372, ΔR2-3: 275-336. FIG. 13c is an image where wild-type 2N4R-tau, single C291A or C322A mutants, and the double C291A C322A (2CA) mutant were evaluated in acetyltransferase assays similar to FIG. 13b. FIG. 13d is an image showing cysteine mutant proteins in full-length 2N4R-tau, tau-K18, or tau-K19 that were analyzed in acetyltransferase activity assays. FIG. 13e is an image showing acetylation immunoblotting of WT and cysteine-mutant tau-K18 and tau-K19 fragments determined by using Ac-K280, Ac-K369, Ac-lysine, and total tau (K9JA) antibodies. FIG. 13f is an image showing acetyltransferase assays of WT and cysteine mutants performed in the absence (lanes 1-4) or presence (lanes 5-8) of active recombinant Creb-binding protein (CBP). FIG. 13g is an image of an immunoblot analysis using Ac-K280 (ac-tau) and total tau antibodies (K9JA). Samples are lysates from QBI-293 cells transfected with tau-K18 containing a P301L mutation (K18-PL) or a comparable mutant lacking Cys291 and Cys322 (K18-PL-2CA) followed by treatment with 3-methyladenine (3MA) or sodium arsenite. Quantification of acetylated tau-K18 was determined as a ratio of acetylated tau (ac-tau) to total tau.

FIG. 14, comprising FIGS. 14a-14c, is a series of images depicting the results of experiments showing that cysteine blocking agents inhibit tau-mediated acetyltransferase activity. FIG. 14a is an image showing that Tau proteins pre-incubated with N-ethyl-maleimide (NEM) or iodoacetamide (IA) exhibited minimal acetyltransferase activity. FIG. 14b is an image showing that 2N4R-tau pre-incubated with increasing concentrations of either tubulin or pre-formed microtubules exhibited inhibition of tau acetylation in acetyltransferase activity assays. FIG. 14c is an image showing bovine brain derived microtubule associated protein (MAP-rich) fractions containing all CNS tau isoforms that were evaluated in acetyltransferase assays in the presence of increased tubulin concentrations followed by immunoblotting with Ac-K280 and total tau (T46) antibodies.

FIG. 15, comprising FIGS. 15a-15e, is a series of images showing kinetic and functional analysis of tau acetyltransferase activity. FIG. 15a is a schematic of the tau-K18 MT-binding region. K18 mutants containing sequential deletions are numbered 1-13 spanning the solid black lines. Catalytic cysteine residues are highlighted in green, phosphorylated KXGS serines in red, and the auto-acetylated tau lysine-280 (K280) in purple. FIG. 15b is a graph depicting Tau-K18 or the cysteine-deficient 2CA and 2CS mutants that were analyzed using filter-binding acetyltransferase assays in the presence of increasing concentrations of [³H]-labeled acetyl-CoA. A saturation plot analysis yielded Kcat and Km values for tau-K18 acetyltransferase activity. FIG. 15c is a graph depicting cofactor inhibition analysis that was performed using increasing concentrations of CoA, acetonyl-CoA, or pantothenic acid and the percent remaining tau acetyltransferase activity was determined followed by assessment of IC₅₀ values for CoA and acetonyl-CoA-mediated inhibition. FIG. 15d is a graph depicting wild-type tau-K18 (black bar), individual S262E (light gray) or S356E (dark gray) phospho-mimic mutants, or the double S262E S356E mutant (2SE, white bar) that were analyzed using filter-binding acetyltransferase assays. The velocities at 800 μM acetyl-CoA were normalized to wild-type to determine fold induction of tau acetyltransferase activity. FIG. 15e is an image of an immunoblot analysis of soluble and insoluble lysates using Ac-K280 (ac-tau) and total tau antibodies (K9JA). Samples are lysates from QBI-293 cells transfected with K18-P301L (K18-PL), the phospho-mimic mutant (K18-PL-2SE), or a control K18-PL containing serine to alanine substitutions (K18-PL-2SA) and treated with 3MA or sodium arsenite, where indicated. All error bars represent standard error of the mean (SEM) from N=3 independent biological replicates. Statistical analysis was performed using an unpaired t-test (*P<0.0125 and ***P<0.0002).

FIG. 16, comprising FIGS. 16a-16d, is a series of images depicting that tau auto-acetylation occurs via intra- and inter-molecular mechanisms. FIG. 16a is a plot of initial rate of auto-acetylation vs. tau-K18 concentration shown for a 30-min reaction. Error bars represent standard error of the mean (SEM) from N=3 independent experiments (see also FIG. 20). FIG. 16b is a schematic of tau-K18-mediated acetylation of full-length 2N4R-tau; the K18-2CA mutant displays impaired acetyltransferase activity towards full-length tau substrates. FIG. 16c is an immunoblot analysis of 2N4R-tau and 2N4R-tau-2CA incubated with increasing concentrations of tau-K18 or tau-K18-2CA. Intermolecular tau acetylation products for full-length 2N4R-tau and tau-K18 were analyzed with the indicated tau antibodies. FIG. 16d is a collection of immunofluorescence images showing double-labeling immunofluorescence using Ac-K280 or total mouse tau (mTau) antibodies used to assess the ability of exogenously added 3R-tau to acetylate endogenous 4R-tau present in mature mouse neurons. Trans-acetylation immunofluorescence was performed by incubating purified tau-K19 (top and middle rows) or tau-K19-C322A mutant (bottom row), with fixed and permeabilized hippocampal neurons. White arrows indicate a few regions of prominent acetylated tau immunoreactivity. Immunofluroescence images are shown in low magnification (top and bottom rows, scale bar=100 μm) and high magnification (middle row, scale bar=25 μm).

FIG. 17, comprising FIGS. 17a-17d, shows the results of an analysis of CBP-mediated acetylation of tau. FIGS. 17 a and 17 b are images of immunoblots where QBI-293 cells were co-transfected with plasmids expressing wild-type tau-K18 or tau-2CA mutant as well as a panel of the indicated HATs (histone acetyltransferases). Cell lysates were analyzed by immunoblotting using antibodies detecting acetylated tau (ac-K280), total tau (K9JA), acetylated tubulin, and acetylated histone H3 (ac-H3K18). Tip60, GCN5, and PCAF proteins are FLAG-tagged and their expression was confirmed using anti-FLAG antibody (M2, Sigma). Note that only CBP and p300 were capable of acetylating wild-type tau-K18 or tau-2CA. The decreased efficiency of p300 compared to CBP was likely due to low level full-length p300 over-expression observed in these cells. FIG. 17c is a graph where the semi-synthetic p300 (ss-p300) catalytic domain was analyzed for its ability to acetylate tau-K18 using filter-binding acetyltransferase assays in the presence of 25 μM [³H]-acetyl-CoA. A saturation plot analysis generated Kcat and Km values for ss-p300 acetyltransferase activity towards tau-K18 substrate. Error bars represent standard error of the mean (SEM). FIG. 17d is a series of images of immunoblots where cells were transfected with K18-PL or K18-PL-2CA plasmids followed by overnight treatment with 10 mM 3-methyladenine (3MA) to induce tau acetylation and 20 mM C646 to block CBP/p300 activity, where indicated. Cell lysates were analyzed by immunoblotting as described in FIG. 17a. To confirm C646 inhibition of CBP/p300, cells were treated overnight with C646 followed by 5 hr incubation with 200 nM Trichostatin-A (TSA), where indicated. Acetylation of the CBP/p300 substrate, histone H3, was analyzed by immunoblotting using the acetylated histone antibodies ac-H3K9 and ac-H3K18.

FIG. 18 is a graph showing kinetic analysis of phospho-mimic tau-K18 mutants. Phospho-mimic serine to glutamic acid substitutions at residues 262, 356, or both (2SE) were assayed for acetyltransferase activity in vitro. An Ac-CoA titration was performed ranging from 2.5 to 800 uM to calculate catalytic parameters. Experiments were performed in triplicate and error bars represent standard error of the mean (SEM).

FIG. 19, comprising FIG. 19a-19i, is a series of images showing that tau is insufficient to promote microtubule acetylation in PTK2 cells. PTK2 epithelial cells were transfected with plasmids expressing wild-type tau-2N4R (FIG. 19a-FIG. 19c and FIG. 19d-FIG. 19f) or HA-tagged human tubulin acetyltransferase a-TAT-1 (FIG. 19g-FIG. 19i) followed by immunostaining analysis using anti-HA, anti-tubulin or anti-acetylated tubulin antibodies. Merged panels indicate extensive a-TAT-1-mediated acetylation of tubulin, as expected, while tau is unable to promote tubulin acetylation in PTK2 cells.

FIG. 20 is a graph showing that Tau-K18 displays first-order reaction kinetics. Filter-binding assays were performed with tau-K18 in the presence of [3H]-acetyl-CoA at the indicated timepoints. A plot of acetylated tau vs. tau concentration at all timepoints was generated to evaluate intra- or inter-molecular acetylation mechanism, as detailed in the methods described in Example 2. Error bars indicate standard error of the mean (SEM) from N=3 independent experiments.

FIG. 21, comprising FIGS. 21a-21i, is a series of images depicting inter-molecular tau auto-acetylation in cultured QBI-293 cells. QBI-293 cells adhered to cover slips were transfected with a 4R-tau (2N4R) expression plasmid and fixed/permeabilized followed by control incubation (a-c) or exposure to purified 3R-tau (0N3R) (d-i) in the presence of either CoA (g-i) or acetyl-CoA (d-f). Cells were subsequently analyzed by immunostaining using Ac-K280, which detects acetylated cellular 4R-tau, and RD4, which detects total ectopically expressed 4R-tau, but not the exogenously added recombinant 3R-tau. Only 3R-tau enzyme in presence of acetyl-CoA was capable of acetylating intracellular 4R-tau substrate.

### DETAILED DESCRIPTION

The present invention relates generally to diagnostic methods and markers, prognostic methods and markers, and therapy evaluators for neurodegenerative disorders. Non-limiting examples of neurodegenerative disorders include, but are not limited to Alzheimer's Disease (AD), corticobasal degeneration (CBD), Pick's disease (PiD), progressive supranuclear palsy, argyrophilic grain disease, tangle predominant senile dementia, Guam parkinsonism-dementia complex, frontotemporal dementia, frontotemporal lobar degeneration, FTDP-17, Lytico-Bodig disease, and Parkinson's disease.

In one embodiment, the biomarker of the present invention is acetylated tau. In one embodiment, the biomarker of the present invention is tau acetylated at one or more of the lysine residues, K150, K163, K174, K234, K240, K259, K274, K280, K281, K290, K311, K369, and K395. It is noted that the residue identifiers of the lysines described herein are in reference to full-length wild type tau. However, as would be understood by those skilled in the art, the biomarkers of the invention are not limited to acetylated residues on full-length wild type tau. Rather, the biomarkers of the invention also include acetylation of like residues, analogous to K150, K163, K174, K234, K240, K259, K274, K280, K281, K290, K311, K369, and K395 of full-length wild type tau, found on other tau isoforms. In one embodiment, a biomarker of the invention is acetylated tau, where tau comprises SEQ ID NO: 4 and the lysine in SEQ ID NO: 4 is acetylated. In one embodiment, a biomarker of the invention is acetylated tau, where tau comprises SEQ ID NO: 3 and the first lysine in SEQ ID NO: 3 is acetylated. In one embodiment, a biomarker of the invention is acetylated tau, where tau comprises SEQ ID NO: 3 and the second lysine in SEQ ID NO: 3 is acetylated. In one embodiment, a biomarker of the invention is acetylated tau, where tau comprises SEQ ID NO: 5 and the first lysine in SEQ ID NO: 5 is acetylated.

In one embodiment, the present invention relates to biomarkers of Alzheimer's Disease, methods for diagnosis of Alzheimer's Disease, methods of determining predisposition to Alzheimer's Disease, methods of monitoring progression/regression of Alzheimer's Disease, methods of assessing efficacy of compositions for treating Alzheimer's Disease, methods of screening compositions for activity in modulating biomarkers of Alzheimer's Disease, methods of treating Alzheimer's Disease, as well as other methods based on biomarkers of Alzheimer's Disease.

In another embodiment, the invention relates to biomarkers that distinguish pathologically confirmed AD from cognitively normal subjects and patients with other neurodegenerative disorders. In one embodiment, the biomarker of the present invention distinguishes between neurodegenerative disorders that differ in their patterns of insoluble tau isoforms present in the disorder. In one embodiment the biomarkers of the present invention discriminate between disorders associated with 4R-tau pathology and disorders without 4R-tau pathology.

In one embodiment, the present invention is related to compositions that detect a biomarker associated with a neurodegenerative disorder. In one embodiment, the compositions of the invention detect acetylated tau. In one embodiment, the compositions detect tau acetylated at one or more of K150, K163, K174, K234, K240, K259, K274, K280, K281, K290, K311, K369, and K395. As discussed elsewhere herein, the biomarkers of the invention also include acetylation of like residues, analogous to K150, K163, K174, K234, K240, K259, K274, K280, K281, K290, K311, K369, and K395 of full-length wild type tau, found on other tau isoforms.

According to the present invention, certain biomarkers are associated with an elevated risk of having or developing a neurodegenerative disorder. Persons so identified have an elevated risk of having or developing a neurodegenerative disorder. Kits useful in practicing embodiments of the inventive methods are also provided.

The invention also provides a method for permitting refinement of disease diagnosis, disease risk prediction, and clinical management of patients associated with a neurodegenerative disorder. That is, the biomarkers of the invention can be used as a marker for the disease state or disease risk. For example, the presence of the selective biomarkers of the invention permits refinement of disease diagnosis, disease risk prediction, and clinical management of patients being treated with agents that are associated with a particular neurodegenerative disorder.

In still further embodiments, the invention provides methods of monitoring a particular biomarker to evaluate the progress of a therapeutic treatment of a neurodegenerative disorder.

The invention also provides methods for screening an individual to determine if the individual is at increased risk of having a neurodegenerative disorder. Individuals found to be at increased risk can be given appropriate therapy and monitored using the methods of the invention.

The invention also provides compositions useful for treating and preventing a neurodegenerative disease. In one embodiment, such therapeutic compounds reduce or prevent tau acetylation. The invention also provides for methods of treating or preventing a neurodegenerative disease. In one embodiment, the methods comprise administering to a subject an effective amount of a therapeutic composition that reduces or prevents tau acetylation?).

### DEFINITIONS

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

The term “abnormal” when used in the context of organisms, tissues, cells or components thereof, refers to those organisms, tissues, cells or components thereof that differ in at least one observable or detectable characteristic (e.g., age, treatment, time of day, etc.) from those organisms, tissues, cells or components thereof that display the “normal” (expected) respective characteristic. Characteristics which are normal or expected for one cell or tissue type, might be abnormal for a different cell or tissue type.

“Amplification” refers to any means by which a polynucleotide sequence is copied and thus expanded into a larger number of polynucleotide sequences, e.g., by reverse transcription, polymerase chain reaction or ligase chain reaction, among others.

The term “biomarker” is a characteristic that is objectively measured and evaluated as an indicator of normal biological processes, pathological processes, or pharmacological responses to a therapeutic intervention. The biomarker can for example describe a substance whose detection indicates a particular disease state. The biomarker may be a peptide that causes disease or is associated with susceptibility to disease. In some instances, the biomarker may refer to a specific post-translational modification, or pattern of modifications, of a peptide (i.e phosphorylation, glycosylation, or acetylation) which distinguishes itself from an otherwise identical peptide. In some instances, the biomarker may be a gene that causes disease or is associated with susceptibility to disease. In other instances, the biomarker is a metabolite. In any event, the biomarker can be differentially present (i.e., increased or decreased) in a biological sample from a subject or a group of subjects having a first phenotype (e.g., having a disease) as compared to a biological sample from a subject or group of subjects having a second phenotype (e.g., not having the disease). A biomarker is preferably differentially present at a level that is statistically significant (i.e., a p-value less than 0.05 and/or a q-value of less than 0.10 as determined using either Welch's T-test or Wilcoxon's rank-sum Test).

The term “body fluids” includes any fluids which can be obtained from a mammalian body. Thus, the term “body fluids” also includes homogenates of any tissues and other body matter. More particularly, however, the term “body fluids” includes fluids that are normally or abnormally secreted by or excreted from the body. The respective fluids may include, but are not limited to: blood, plasma, lymph, urine, and cerebrospinal fluid, blood, plasma, and cerebrospinal fluid.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

A disease or disorder is “alleviated” if the severity of a symptom of the disease or disorder, the frequency with which such a symptom is experienced by a patient, or both, is reduced.

An “effective amount” or “therapeutically effective amount” of a compound is that amount of compound which is sufficient to provide a beneficial effect to the subject to which the compound is administered. An “effective amount” of a delivery vehicle is that amount sufficient to effectively bind or deliver a compound.

As used herein, an “instructional material” includes a publication, a recording, a diagram, or any other medium of expression which can be used to communicate the usefulness of a compound, composition, vector, or delivery system of the invention in the kit for effecting alleviation of the various diseases or disorders recited herein. Optionally, or alternately, the instructional material can describe one or more methods of alleviating the diseases or disorders in a cell or a tissue of a mammal. The instructional material of the kit of the invention can, for example, be affixed to a container which contains the identified compound, composition, vector, or delivery system of the invention or be shipped together with a container which contains the identified compound, composition, vector, or delivery system. Alternatively, the instructional material can be shipped separately from the container with the intention that the instructional material and the compound be used cooperatively by the recipient.

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell or a test tube.

The term “microarray” refers broadly to both “DNA microarrays” and “DNA chip(s),” and encompasses all art-recognized solid supports, and all art-recognized methods for affixing nucleic acid molecules thereto or for synthesis of nucleic acids thereon.

“Naturally occurring” as used herein describes a composition that can be found in nature as distinct from being artificially produced. For example, a nucleotide sequence present in an organism, which can be isolated from a source in nature and which has not been intentionally modified by a person in the laboratory, is naturally occurring.

As used herein, “phenotypically distinct” is used to describe organisms, tissues, cells or components thereof, which can be distinguished by one or more characteristics, observable and/or detectable by current technologies. Each of such characteristics may also be defined as a parameter contributing to the definition of the phenotype. Wherein a phenotype is defined by one or more parameters an organism that does not conform to one or more of the parameters shall be defined to be distinct or distinguishable from organisms of the phenotype.

The terms “patient,” “subject,” “individual,” and the like are used interchangeably herein, and refer to any animal, or cells thereof whether in vitro or in situ, amenable to the methods described herein. In certain non-limiting embodiments, the patient, subject or individual is a human.

A “prophylactic” treatment is a treatment administered to a subject who does not exhibit signs of a disease or exhibits only early signs of the disease for the purpose of decreasing the risk of developing pathology associated with the disease.

The term “protein” typically refers to large polypeptides.

“Sample” or “biological sample” as used herein means a biological material isolated from a subject. The biological sample may contain any biological material suitable for detecting the desired biomarkers, and may comprise cellular and/or non-cellular material from the subject. The sample can be isolated from any suitable biological tissue or fluid such as, for example, blood, blood plasma, urine, or cerebral spinal fluid (CSF).

The terms “marker” and “epigenetic marker” are used interchangeably herein to refer to a distinguishing or characteristic substance that may be found in a biological material. The substance may, for example, be a protein, an enzyme, an RNA molecule or a DNA molecule. Non-limiting examples of such a substance include a kinase, a methylase, and an acetylase. The terms also refer to a specific characteristic of the substance, such as, but not limited to, a specific phosphorylation, methylation, or acetylation event or pattern, making the substance distinguishable from otherwise identical substances. The terms further refer to a specific modification, event or step occurring in a signaling pathway or signaling cascade, such as, but not limited to, the deposition or removal of a specific phosphate, methyl, or acetyl group.

The term to “treat,” as used herein, means reducing the frequency with which symptoms are experienced by a patient or subject or administering an agent or compound to reduce the frequency with which symptoms are experienced.

A “therapeutic” treatment is a treatment administered to a subject who exhibits signs of pathology, for the purpose of diminishing or eliminating those signs.

As used herein, “treating a disease or disorder” means reducing the frequency with which a symptom of the disease or disorder is experienced by a patient. Disease and disorder are used interchangeably herein.

The phrase “therapeutically effective amount,” as used herein, refers to an amount that is sufficient or effective to prevent or treat (delay or prevent the onset of, prevent the progression of, inhibit, decrease or reverse) a disease or condition associated with tau aggregation or other tau pathology, including alleviating symptoms of such diseases.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### Description

The present invention relates to the identification of biomarkers that are associated with neurodegenerative disorders. In one embodiment, the biomarkers of the present invention are useful for diagnosing a neurodegenerative disorder. In another embodiment, the biomarkers of the present invention are useful for assessing the risk of a subject for developing a neurodegenerative disorder. In yet another embodiment, the biomarkers of the present invention are useful to distinguish between neurodegenerative disorders. Non-limiting examples of neurodegenerative disorders in which the present invention is useful include Alzheimer's Disease (AD), corticobasal degeneration (CBD), Pick's disease (PiD), progressive supranuclear palsy, argyrophilic grain disease, tangle predominant senile dementia, Guam parkinsonism-dementia complex, frontotemporal dementia, frontotemporal lobar degeneration, FTDP-17, Lytico-Bodig disease, and Parkinson's disease.

In one embodiment, the biomarkers of the present invention comprise acetylated tau. In one embodiment, the biomarkers of the present invention comprise tau acetylated at one or more of the following lysine residues: K150, K163, K174, K234, K240, K259, K274, K280, K281, K290, K311, K369, and K395. As discussed elsewhere herein, the biomarkers of the invention also include acetylation of like residues, analogous to K150, K163, K174, K234, K240, K259, K274, K280, K281, K290, K311, K369, and K395 of full-length wild type tau, found on other tau isoforms).

Such biomarkers could be used for neurodegenerative disorder screening and diagnosis, as well as potentially for assessing response to new therapies. Given the probability of multiple underlying pathogenic mechanisms of some neurodegenerative disorders, the present invention provides novel biomarkers present in the bodily fluid of a subject. The biomarkers of the invention allow a more accurate diagnosis or prognosis of a neurodegenerative disorder.

The biomarkers of the invention may also allow the monitoring of a neurodegenerative disorder, such that a comparison of biomarker levels allows an evaluation of disease progression in subjects that have been diagnosed with a neurodegenerative disorder, or that do not yet show any clinical signs of the neurodegenerative disorder. Moreover, the biomarkers of the invention may be used in concert with known biomarkers such that a more accurate diagnosis or prognosis of the neurodegenerative disorder may be made.

Generally, the invention provides that biomarkers are determined for biological samples from human subjects diagnosed with a neurodegenerative disorder, for example Alzheimer's Disease, as well as from one or more other groups of human subjects (e.g., healthy control subjects not diagnosed with Alzheimer's Disease). The biomarkers for a particular neurodegenerative disorder are compared to the biomarkers for biological samples from the one or more other groups of subjects. Those molecules differentially present, including those molecules differentially present at a level that is statistically significant, in the profile of a neurodegenerative disorder sample as compared to another group (e.g., healthy control subjects not diagnosed with Alzheimer's Disease) are identified as biomarkers to distinguish those groups.

The biomarkers disclosed herein may be used in combination with existing clinical diagnostic measures of Alzheimer's Disease and/or other neurodegenerative diseases. Combinations with clinical diagnostics may facilitate the disclosed methods, or confirm results of the disclosed methods (for example, facilitating or confirming diagnosis, monitoring progression or regression, and/or determining predisposition to Alzheimer's Disease and/or neurodegenerative disorders).

The biomarkers of the invention can be used to facilitate the optimum selection of treatment protocols, and open new venues for the development of effective therapy for a desired neurodegenerative disorder. Biomarkers of the invention can be used to guide treatment selection for individual patients, as well as to guide the development of new therapies specific to each type of neurodegenerative disorder.

### Tau

Neuritic plaques, neurofibrillary tangles (NFTs), and neuropil threads are hallmark lesions of Alzheimer's disease (AD) that contain filamentous intraneuronal inclusions of tau protein (Buee et al., Brain Res. Rev. 33: 95-130 (2000)). Because tau filaments form in brain regions associated with memory retention, and because their appearance correlates well with the degree of dementia, they have emerged as robust markers of disease progression (Braak et al., Acta. Neuropathol. (Berl) 87: 554-567 (1991); Braak et al., Acta Neuropathol. (Berl) 87: 554-567 (1994)). Tau filaments also appear in other neurodegenerative tauopathies, including Pick's disease and corticobasal degeneration, with the neuronal populations affected being disease dependent (Feany et al., Ann. Neurol. 87: 554-567 (1996)). Thus tau filament formation coincides with cytoskeletal disorganization that could occur in degenerating neurons, and may represent a fundamental pathophysiological response of neurons to various insults.

NFTs have long been recognized as the hallmarks of Alzheimer's disease and the existence of a close correlation between the presence and distribution of NFTs and the degree of cognitive impairment in Alzheimer's disease further emphasizes the critical role of tau pathology in the development of the disease. Hyperphosphorylated tau proteins tend to dissociate from microtubule and assemble into paired helical filaments. Other factors proposed to facilitate the aggregation of tau include oxidation, polyanions, and nucleation. In vitro tests have demonstrated that all tau isoforms are able to aggregate; however, tau fragments containing the MT-binding repeat regions exhibit faster kinetics using in vitro assembly assays. Thus, not wishing to be bound by a theory, N- and C-terminal truncation of tau could be a significant factor that enhances the aggregation of tau and causes the generation of tangle like structures.

Accumulation of early-stage oligomeric aggregated tau species is associated with the development of functional deficits during the pathogenic progression of tauopathy, and accumulation of pre-fibril granular oligomers correlates with Braak staging in post-mortem analysis of AD brains (Berger Z, et al. (2007) J. Neurosci. 27(14): 3650-62; Maeda S, et al. (2006) Neuroscience Res. 54:197-201). The role of misfolded tau in AD has been shown in a number of studies using antibodies specific for tau conformational epitopes. Levels of conformationally altered tau in postmortem neocortical specimen increased with progressing dementia in AD (Haroutunian V, et al. (2007) Neurobiology of Aging, 28: 1-7). The involvement of soluble tau as the primary causative factor of neurotoxicity in AD is supported by these findings. Tau has also been found to associate with Aβ (beta amyloid plaque) in brain tissue, and this interaction could facilitate the co-aggregation of these proteins (Guo J-P et al. (2006) PNAS 103:1953-1958).

Tau protein exists in six isoforms in the adult brain (Goedert et al. (1989) Neuron, 3, 519-526). The term “tau protein” refers to any protein of the tau protein family including, but not limited to, native tau protein monomer, precursor tau proteins, tau peptides, tau intermediates, metabolites, tau derivatives that can be antigenic, or antigenic fragments thereof. Fragments include less than entire tau protein provided the fragment is antigenic and will cause antibodies or antibody binding fragments to react with the tau fragment.

The amino acid sequences of tau protein isoforms are also described in Ballatore C, Lee V M, Trojanowski J Q, Nat Rev Neurosci. 2007 September; 8(9):663-72. Review; Hasegawa M., Neuropathology. 2006 October; 26(5):484-90; and D'Souza I, and Schellenberg G D, Biochim Biophys Acta. 2005 Jan. 3; 1739(2-3):104-15. These references, mentioned in the specification, are incorporated herein by reference for all that they disclose.

In some cases, mutations in the gene encoding Tau (sometimes called MAPT) cause tauopathies, particularly in frontotemporal dementia and Parkinsonism linked to chromosome 17 (FTDP-17) and other frontotemporal dementias. Many FTDP-17 mutations decrease binding to microtubules in vitro and/or increase their propensity to form fibrils. Other tauopathy-associated mutations alter the splice pattern of Tau to generate predominantly 3R or 4R Tau. Yet another class of Tau mutations on the N-terminus alters the ability to bind to dynactin. All of these mutations have the potential to interfere with normal functions of Tau.

The present invention provides compositions and methods useful for diagnosing a neurodegenerative disorder, assessing the risk for a subject to develop a neurodegenerative disorder, distinguishing between neurodegenerative disorders, assessing the efficacy of a treatment for a neurodegenerative disorder, and the like. The present invention is based on the discovery that acetylated tau is a specific biomarker detected in AD and other neurodegenerative disorders. It is further demonstrated herein, that tau acetylation inhibits tau function via impaired tau-microtubule interactions and promotes pathological tau aggregation. Specific lysine residues, including K150, K163, K174, K234, K240, K259, K274, K280, K281, K290, K311, K369, and K395, were identified as the major sites of tau acetylation. It is noted that the residue identifiers of the lysines described herein are in reference to full-length wild type tau. However, as would be understood by those skilled in the art, the biomarkers of the invention are not limited to acetylated residues on full-length wild type tau. Rather, the biomarkers of the invention also include acetylation of like residues, analogous to K150, K163, K174, K234, K240, K259, K274, K280, K281, K290, K311, K369, and K395 of full-length wild type tau, found on other tau isoforms. In one embodiment, a biomarker of the invention is acetylated tau, where tau comprises SEQ ID NO: 4 and the lysine in SEQ ID NO: 4 is acetylated. In one embodiment, a biomarker of the invention is acetylated tau, where tau comprises SEQ ID NO: 3 and the first lysine in SEQ ID NO: 3 is acetylated. In one embodiment, a biomarker of the invention is acetylated tau, where tau comprises SEQ ID NO: 3 and the second lysine in SEQ ID NO: 3 is acetylated. In one embodiment, a biomarker of the invention is acetylated tau, where tau comprises SEQ ID NO: 5 and the first lysine in SEQ ID NO: 5 is acetylated.

The present invention is also based upon the development of an antibody that specifically recognizes tau K280 acetylation. Immunohistochemical and biochemical studies of brains from tau transgenic mice and patients with AD and related tauopathies showed that acetylated K280 tau pathology is specifically associated with insoluble, Thioflavin-positive tau aggregates. It was found that tau K280 acetylation was only detected in diseased tissue, suggesting it may have a role in pathological tau transformation and is a specific biomarker for AD and other neurodegenerative disorders.

### Compositions

In one aspect, the present invention relates to compositions that specifically detect a biomarker associated with a neurodegenerative disorder. As detailed elsewhere herein, the present invention is based upon the finding that acetylated tau is a specific biomarker for AD and other neurodegenerative disorders. In one embodiment, the compositions of the invention specifically bind to and detect acetylated tau. In one embodiment the compositions of the invention specifically bind to and detect tau acetylated at one or more of K150, K163, K174, K234, K240, K259, K274, K280, K281, K290, K311, K369, and K395. As discussed elsewhere herein, the biomarkers of the invention also include acetylation of like residues, analogous to K150, K163, K174, K234, K240, K259, K274, K280, K281, K290, K311, K369, and K395 of full-length wild type tau, found on other tau isoforms.

In one embodiment, the compositions of the invention specifically bind to tau acetylated at K280 (K280 tau acetylation). The composition of the present invention can comprise an antibody, a peptide, a small molecule, a nucleic acid, and the like.

In one embodiment, the composition comprises an antibody, where the antibody specifically binds to a biomarker of the invention. The term “antibody” as used herein is intended to include fragments thereof which are also specifically reactive with a biomarker. Antibodies can be fragmented using conventional techniques and the fragments screened for utility in the same manner as described above for whole antibodies. For example, F(ab)₂ fragments can be generated by treating antibody with pepsin. The resulting F(ab)₂ fragment can be treated to reduce disulfide bridges to produce Fab fragments. Antigen-binding portions may also be produced by recombinant DNA techniques or by enzymatic or chemical cleavage of intact antibodies. Antigen-binding portions include, inter alia, Fab, Fab′, F(ab′)2, Fv, dAb, and complementarity determining region (CDR) fragments, single-chain antibodies (scFv), single domain antibodies, bispecific antibodies, chimeric antibodies, humanized antibodies, diabodies and polypeptides that contain at least a portion of an immunoglobulin that is sufficient to confer specific antigen binding to the polypeptide. In certain embodiments, the antibody further comprises a label attached thereto and able to be detected (e.g., the label can be a radioisotope, fluorescent compound, enzyme or enzyme co-factor).

In certain embodiments, an antibody of the invention is a monoclonal antibody, and in certain embodiments, the invention makes available methods for generating novel antibodies that specifically bind the biomarker of the invention. For example, a method for generating a monoclonal antibody that specifically binds a biomarker, may comprise administering to a mouse an amount of an immunogenic composition comprising the biomarker, or fragment thereof, effective to stimulate a detectable immune response, obtaining antibody-producing cells (e.g., cells from the spleen) from the mouse and fusing the antibody-producing cells with myeloma cells to obtain antibody-producing hybridomas, and testing the antibody-producing hybridomas to identify a hybridoma that produces a monocolonal antibody that binds specifically to the biomarker. Once obtained, a hybridoma can be propagated in a cell culture, optionally in culture conditions where the hybridoma-derived cells produce the monoclonal antibody that binds specifically to the biomarker. The monoclonal antibody may be purified from the cell culture.

The term “specifically reactive with” as used in reference to an antibody is intended to mean, as is generally understood in the art, that the antibody is sufficiently selective between the antigen of interest (e.g., a biomarker) and other antigens that are not of interest. In certain methods employing the antibody, such as therapeutic applications, a higher degree of specificity in binding may be desirable. Monoclonal antibodies generally have a greater tendency (as compared to polyclonal antibodies) to discriminate effectively between the desired antigens and cross-reacting polypeptides. One characteristic that influences the specificity of an antibody:antigen interaction is the affinity of the antibody for the antigen. Although the desired specificity may be reached with a range of different affinities, generally preferred antibodies will have an affinity (a dissociation constant) of about 10⁻⁶, 10⁻⁷, 10⁻⁸, 10⁻⁹ or less.

In addition, the techniques used to screen antibodies in order to identify a desirable antibody may influence the properties of the antibody obtained. A variety of different techniques are available for testing interaction between antibodies and antigens to identify particularly desirable antibodies. Such techniques include ELISAs, surface plasmon resonance binding assays (e.g., the Biacore binding assay, Biacore AB, Uppsala, Sweden), sandwich assays (e.g., the paramagnetic bead system of IGEN International, Inc., Gaithersburg, Md.), western blots, immunoprecipitation assays, immunocytochemistry, and immunohistochemistry.

In one aspect, the present invention relates to compositions used for treating or preventing a neurodegenerative disorder. As detailed elsewhere herein, the present invention is based upon the findings that tau acetylation impairs microtubule stabilization, and promotes tau aggregation and fibrillization, all of which are known pathological events in the development of a variety of neurodegenerative disorders. Therefore, in one embodiment, the present invention provides compositions that prevent tau acetylation. In one embodiment, the compositions prevent tau acetylation at one or more of K150, K163, K174, K234, K240, K259, K274, K280, K281, K290, K311, K369, and K395. In another embodiment, the present invention provides compositions that reduce tau acetylation. In one embodiment, the compositions reduce tau acetylation at one or more of K150, K163, K174, K234, K240, K259, K274, K280, K281, K290, K311, K369, and K395. As discussed elsewhere herein, the biomarkers of the invention also include acetylation of like residues, analogous to K150, K163, K174, K234, K240, K259, K274, K280, K281, K290, K311, K369, and K395 of full-length wild type tau, found on other tau isoforms.

Compositions useful for preventing and/or reducing tau acetylation include proteins, peptides, nucleic acids, small molecules, and the like. For example, in one embodiment, the composition is a nucleic acid (e.g. siRNA) targeted to reduce the expression of one or more acetyl-transferases. In another embodiment, the composition is a nucleic acid that increases the expression of deacetylases (e.g. HDACs). In another embodiment, the composition inhibits or reduces the activity of acetyl-transferases. In yet another embodiment, the composition increases the activity of deacetylases (e.g. HDACs).

### Methods

In one aspect, the present invention relates to methods of assessing a neurodegenerative disorder. As detailed elsewhere herein, the present invention is based upon the finding that acetylated tau is a specific biomarker for AD and other neurodegenerative disorders.

Non-limiting examples of neurodegenerative disorders in which the methods of the present invention are useful include Alzheimer's Disease (AD), corticobasal degeneration (CBD), Pick's disease (PiD), progressive supranuclear palsy, argyrophilic grain disease, tangle predominant senile dementia, Guam parkinsonism-dementia complex, frontotemporal dementia, frontotemporal lobar degeneration, FTDP-17, Lytico-Bodig disease, and Parkinson's disease.

In one embodiment, the biomarkers of the invention are useful for diagnosis of a neurodegenerative disorder and permits refinement of disease diagnosis, disease risk prediction, and clinical management of patients. Thus, the invention provides a method of improving the choice of treatment options and prognosis of a patient having a neurodegenerative disorder. In some instances, the biomarkers are useful for evaluating the effects of potential and applied therapies.

The biomarkers of the invention provide a method of early diagnosis of a neurodegenerative disorder. In some instances, the biomarkers of the invention can distinguish between the possible underlying diseases responsible for neurodegeneration.

In still further embodiments, the invention provides methods of monitoring a particular biomarker to evaluate the progress of a therapeutic treatment of a neurodegenerative disorder.

The invention also provides methods for screening an individual to determine if the individual is at increased risk of having a neurodegenerative disorder, or of developing a neurodegenerative disorder. Individuals found to be at increased risk can be given appropriate therapy and monitored using the methods of the invention.

In one embodiment, a biomarker of the invention is typically a protein, the level of which varies with disease state and may be readily quantified. In one embodiment, the biomarker is a post-translational modification of a protein, for example the phosphorylation, acetylation, glycosylation, and the like, of a protein. The quantified level may then be compared to a known value. The comparison may be used for several different purposes, including but not limited to, diagnosis of a disorder, prognosis of a disorder, and monitoring treatment of a disorder.

In one embodiment, the present invention includes methods of distinguishing between neurodegenerative disorders. For example, the present invention provides that K280 tau acetylation is present in disorders with 4R-tau present in the insoluble fraction and/or in tau inclusions (e.g. AD, CBD). Therefore, based upon the known, or yet to be known, patterns of tau isoforms present in the pathology of various neurodegenerative disorders, the detection of K280 tau acetylation can distinguish between disorders. Since K280 tau acetylation is not detectable in disorders with predominately 3R-tau pathology (e.g. PiD), as described elsewhere herein, the presence or absence of K280 tau acetylation can be used to refine or rule out individual neurodegenerative disorders.

Generally, any suitable method may be used to analyze the biological sample in order to determine the level(s) of the one or more biomarkers in the sample. Suitable methods include those described elsewhere herein, as well as chromatography (e.g., HPLC, gas chromatography, liquid chromatography), mass spectrometry (e.g., MS, MS-MS), enzyme-linked immunosorbent assay (ELISA), antibody linkage, other immunochemical techniques, and combinations thereof. Further, the level(s) of the one or more biomarkers may be measured indirectly, for example, by using an assay that measures the level of a compound (or compounds) that correlates with the level of the biomarker(s) that are desired to be measured.

The methods of the present invention comprise detecting the level of a biomarker in a biological sample from a subject. In one embodiment, the biological sample is central nervous system tissue. Central nervous system tissue includes brain tissue, spinal cord tissue, cerebral spinal fluid (CSF). Brain tissue can comprise any tissue of the brain, including but not limited to tissues of the cortex, frontal lobe, prefrontal lobe, parietal lobe, occipital lobe, hippocampus, cerebrum, cerebellum, thalamus, pons, hypothalamus, and amygdala. In one embodiment, the biological sample is a bodily fluid. Non-limiting examples of bodily fluid include whole blood, plasma, serum, bile, lymph, pleural fluid, semen, saliva, sweat, urine, and CSF.

As will be appreciated by a skilled artisan, the method of obtaining a biological sample from a subject can and will vary depending upon the nature of the biological sample. Any of a variety of methods generally known in the art may be utilized to obtain a biological sample from a subject. Generally speaking, the method preferably maintains the integrity of the biomarkers of the invention such that it can be accurately quantified in the biological sample.

A biological sample may be tested from any mammal known to suffer from a neurodegenerative disorder (e.g., AD) or used as a disease model for a neurodegenerative disorder (e.g., AD). In one embodiment, the subject is a rodent including, but is not limited to, mice, rats, and guinea pigs. In another embodiment, the subject is a primate including, but is not limited to monkeys, apes, and humans. In an exemplary embodiment, the subject is a human. In some embodiments, the subject has no clinical signs of a neurodegenerative disorder (e.g., AD). In other embodiments, the subject has mild clinical signs of a neurodegenerative disorder (e.g., AD). In yet other embodiments, the subject may be at risk for a neurodegenerative disorder (e.g., AD). In still other embodiments, the subject has been diagnosed with a neurodegenerative disorder (e.g., AD).

The level of the biomarker may encompass the amount of protein, the concentration of protein, or the amount of enzymatic activity. In either embodiment, the level is quantified, such that a value, an average value, or a range of values is determined. In one embodiment, the level of protein concentration of the AD biomarker is quantified. There are numerous known methods and kits for measuring the amount or concentration of a protein in a sample, including ELISA, western blot, absorption measurement, colorimetric determination, Lowry assay, Bicinchoninic acid assay, or a Bradford assay.

The amount or concentration of a protein in a sample can also be analyzed using the methods disclosed herein. For example, the biomarkers of the invention can be identified by logistic regression (LR) analysis, random forest (RF) classification, and predictive analysis of microarrays (PAM). In some instances, biomarkers of the invention can be identified using Multi-Analyte Profile (MAP) analysis. The methods of the invention provides for high sensitivity and specificity with increased accuracy for detecting neurodegenerative disorders.

In another embodiment, the level of enzymatic activity of the biomarker is quantified. Generally, enzyme activity may be measured by means known in the art, such as measurement of product formation, substrate degradation, or substrate concentration, at a selected point(s) or time(s) in the enzymatic reaction. There are numerous known methods and kits for measuring enzyme activity. For example, see U.S. Pat. No. 5,854,152. Some methods may require purification of the biomarker prior to measuring the enzymatic activity of the biomarker. A pure biomarker constitutes at least about 90%, preferably, 95% and even more preferably, at least about 99% by weight of the total protein in a given sample. Biomarkers of the invention may be purified according to methods known in the art, including, but not limited to, ion-exchange chromatography, size-exclusion chromatography, affinity chromatography, differential solubility, differential centrifugation, and HPLC.

In one embodiment, the invention encompasses a method for detecting a neurodegenerative disorder comprising quantifying the level of a neurodegenerative disorder biomarker in a bodily fluid of a subject and subsequently determining if the quantified level of the biomarker is elevated or depressed in comparison to the average level of the biomarker for an otherwise normal subject. The subject may have no clinical signs of a neurodegenerative disorder, the subject might be at risk for a neurodegenerative disorder, or alternatively, the subject might show mild dementia.

An elevated or depressed biomarker level may lead to either a diagnosis or prognosis of a neurodegenerative disorder. In one embodiment, an elevated biomarker level indicates a diagnosis of a neurodegenerative disorder. In another embodiment, an elevated biomarker level indicates a prognosis of a neurodegenerative disorder. In yet another embodiment, a depressed biomarker level indicates a diagnosis of a neurodegenerative disorder. In still yet another embodiment, a depressed biomarker level indicates a prognosis of a neurodegenerative disorder.

The percent elevation or depression of a neurodegenerative disorder biomarker compared to the average level of the biomarker for a normal subject is typically greater than 15% to indicate a diagnosis or prognosis of a neurodegenerative disorder. In some instances, the percent elevation or depression is 15%, 16%, 17%, 18%, 19%, 20%, 21%, or 22%. In other instances, the percent elevation or depression is 23%, 24%, 25%, 26%, 27%, 28%, 29% or 30%. In still other instances, the percent elevation or depression is greater than 30%. In alternative instances, the percent elevation or depression is greater than 50%.

Another embodiment of the invention encompasses a method for monitoring a neurodegenerative disorder comprising quantifying the level of a neurodegenerative disorder biomarker in a biological sample of a subject and comparing the quantified level of the biomarker to a previously quantified biomarker level of the subject to determine if the quantified level is elevated or depressed in comparison to the previous level. The subject may be diagnosed with a neurodegenerative disorder, or alternatively, may have no clinical signs of a neurodegenerative disorder. The comparison may give an indication of disease progression. Therefore, the comparison may serve to measure the effectiveness of a chosen therapy. Alternatively, the comparison may serve to measure the rate of disease progression.

In the context of monitoring a neurodegenerative disorder, the percent elevation or depression of a neurodegenerative disorder biomarker compared to a previous level may be from 0% to greater than about 50%. In one embodiment, the percent elevation or depression is from about 1% to about 10%. In another embodiment, the percent elevation or depression is from about 10% to about 20%. In yet another embodiment, the percent elevation or depression is from about 20% to about 30%. In still another embodiment, the percent elevation or depression is from about 30% to about 40%. In yet still another embodiment, the percent elevation or depression is from about 40% to about 50%. In a further embodiment, the percent elevation or depression is greater than 50%.

Another aspect of the invention encompasses kits for detecting or monitoring a neurodegenerative disorder in a subject. A variety of kits having different components are contemplated by the current invention. Generally speaking, the kit will include the means for quantifying one or more biomarkers in a subject. In another embodiment, the kit will include means for collecting a biological sample, means for quantifying one or more biomarkers in the biological sample, and instructions for use of the kit contents. In certain embodiments, the kit comprises a means for quantifying biomarker enzyme activity. Preferably, the means for quantifying biomarker enzyme activity comprises reagents necessary to detect the biomarker enzyme activity. In certain aspects, the kit comprises a means for quantifying the amount of a biomarker. Preferably, the means for quantifying the amount of a biomarker comprises reagents necessary to detect the amount of a biomarker.

In another aspect, the present invention provides methods for treating or preventing a neurodegenerative disorder. In one embodiment, the methods comprise administering an effective amount of a therapeutic composition to a subject, where the therapeutic composition prevents or reduces tau acetylation. In one embodiment, the methods comprise administering an effective amount of a therapeutic composition to a subject, where the therapeutic composition prevents or reduces tau acetylation at one or more of K150, K163, K174, K234, K240, K259, K274, K280, K281, K290, K311, K369, and K395 as well as acetylation of like residues, analogous to K150, K163, K174, K234, K240, K259, K274, K280, K281, K290, K311, K369, and K395 of full-length wild type tau, found on other tau isoforms. Non-limiting examples of neurodegenerative disorders where compositions that reduce or prevent tau acetylation would be effective include Alzheimer's Disease (AD), corticobasal degeneration (CBD), progressive supranuclear palsy, argyrophilic grain disease, tangle predominant senile dementia, Guam parkinsonism-dementia complex, frontotemporal dementia, frontotemporal lobar degeneration, FTDP-17, Lytico-Bodig disease, Pick's Disease, and Parkinson's disease.

Therapeutic compositions of the invention can be administered to a subject or patient through any means known in the art. Administration of the therapeutic composition in accordance with the present invention may be continuous or intermittent, depending, for example, upon the recipient's physiological condition, whether the purpose of the administration is therapeutic or prophylactic, and other factors known to skilled practitioners. The administration of the compositions of the invention may be essentially continuous over a preselected period of time or may be in a series of spaced doses. Both local and systemic administration is contemplated. The amount administered will vary depending on various factors including, but not limited to, the composition chosen, the particular disease, the weight, the physical condition, and the age of the mammal, and whether prevention or treatment is to be achieved. Such factors can be readily determined by the clinician employing animal models or other test systems which are well known to the art

As contemplated elsewhere herein, compositions of the invention that prevent or reduce tau acetylation may comprise nucleic acids, including DNA and RNA sequences. Pharmaceutical formulations, dosages and routes of administration for nucleic acids are generally disclosed, for example, in Felgner et al., 1997, U.S. Pat. No. 5,580,859. Further, administration of proteins, peptides, siRNA and other compositions that display therapeutic benefit may be accomplished through administration of nucleic acid molecules that encode for such compositions (see, for example, Felgner et al. 1987, U.S. Pat. No. 5,580,859 and Molling et al. 2006, 7,141,550). One or more suitable unit dosage forms having the therapeutic composition(s) of the invention, which, as discussed below, may optionally be formulated for sustained release (for example using microencapsulation, see WO 94/07529, and U.S. Pat. No. 4,962,091 the disclosures of which are incorporated by reference herein), can be administered by a variety of routes including parenteral, including by intravenous and intramuscular routes, as well as by direct injection into the diseased tissue. For example, the therapeutic composition may be directly injected into the brain. The formulations may, where appropriate, be conveniently presented in discrete unit dosage forms and may be prepared by any of the methods well known to pharmacy. Such methods may include the step of bringing into association the therapeutic agent with liquid carriers, solid matrices, semi-solid carriers, finely divided solid carriers or combinations thereof, and then, if necessary, introducing or shaping the product into the desired delivery system.

When the therapeutic compositions of the invention are prepared for administration, they are preferably combined with a pharmaceutically acceptable carrier, diluent or excipient to form a pharmaceutical formulation, or unit dosage form. The total active ingredients in such formulations include from 0.1 to 99.9% by weight of the formulation. A “pharmaceutically acceptable” is a carrier, diluent, excipient, and/or salt that is compatible with the other ingredients of the formulation, and not deleterious to the recipient thereof. The active ingredient for administration may be present as a powder or as granules; as a solution, a suspension or an emulsion.

Pharmaceutical formulations containing the therapeutic compositions of the invention can be prepared by procedures known in the art using well known and readily available ingredients. The therapeutic compositions of the invention can also be formulated as solutions appropriate for parenteral administration, for instance by intramuscular, subcutaneous or intravenous routes.

The pharmaceutical formulations of the therapeutic compositions of the invention can also take the form of an aqueous or anhydrous solution or dispersion, or alternatively the form of an emulsion or suspension.

Thus, the therapeutic composition may be formulated for parenteral administration (e.g., by injection, for example, bolus injection or continuous infusion) and may be presented in unit dose form in ampules, pre-filled syringes, small volume infusion containers or in multi-dose containers with an added preservative. The active ingredients may take such forms as suspensions, solutions, or emulsions in oily or aqueous vehicles, and may contain formulatory agents such as suspending, stabilizing and/or dispersing agents. Alternatively, the active ingredients may be in powder form, obtained by aseptic isolation of sterile solid or by lyophilization from solution, for constitution with a suitable vehicle, e.g., sterile, pyrogen-free water, before use.

It will be appreciated that the unit content of active ingredient or ingredients contained in an individual aerosol dose of each dosage form need not in itself constitute an effective amount for treating the particular indication or disease since the necessary effective amount can be reached by administration of a plurality of dosage units. Moreover, the effective amount may be achieved using less than the dose in the dosage form, either individually, or in a series of administrations.

The pharmaceutical formulations of the present invention may include, as optional ingredients, pharmaceutically acceptable carriers, diluents, solubilizing or emulsifying agents, and salts of the type that are well-known in the art. Specific non-limiting examples of the carriers and/or diluents that are useful in the pharmaceutical formulations of the present invention include water and physiologically acceptable buffered saline solutions, such as phosphate buffered saline solutions pH 7.0-8.0.

The expression vectors, transduced cells, polynucleotides and polypeptides (active ingredients) of this invention can be formulated and administered to treat a variety of disease states by any means that produces contact of the active ingredient with the agent's site of action in the body of the organism. They can be administered by any conventional means available for use in conjunction with pharmaceuticals, either as individual therapeutic active ingredients or in a combination of therapeutic active ingredients. They can be administered alone, but are generally administered with a pharmaceutical carrier selected on the basis of the chosen route of administration and standard pharmaceutical practice.

In general, water, suitable oil, saline, aqueous dextrose (glucose), and related sugar solutions and glycols such as propylene glycol or polyethylene glycols are suitable carriers for parenteral solutions. Solutions for parenteral administration contain the active ingredient, suitable stabilizing agents and, if necessary, buffer substances. Antioxidizing agents such as sodium bisulfate, sodium sulfite or ascorbic acid, either alone or combined, are suitable stabilizing agents. Also used are citric acid and its salts and sodium ethylenediaminetetraacetic acid (EDTA). In addition, parenteral solutions can contain preservatives such as benzalkonium chloride, methyl- or propyl-paraben and chlorobutanol. Suitable pharmaceutical carriers are described in Remington's Pharmaceutical Sciences, a standard reference text in this field.

The active ingredients of the invention may be formulated to be suspended in a pharmaceutically acceptable composition suitable for use in mammals and in particular, in humans. Such formulations include the use of adjuvants such as muramyl dipeptide derivatives (MDP) or analogs that are described in U.S. Pat. Nos. 4,082,735; 4,082,736; 4,101,536; 4,185,089; 4,235,771; and 4,406,890. Other adjuvants, which are useful, include alum (Pierce Chemical Co.), lipid A, trehalose dimycolate and dimethyldioctadecylammonium bromide (DDA), Freund's adjuvant, and IL-12. Other components may include a polyoxypropylene-polyoxyethylene block polymer (Pluronic®), a non-ionic surfactant, and a metabolizable oil such as squalene (U.S. Pat. No. 4,606,918).

Additionally, standard pharmaceutical methods can be employed to control the duration of action. These are well known in the art and include control release preparations and can include appropriate macromolecules, for example polymers, polyesters, polyamino acids, polyvinyl, pyrolidone, ethylenevinylacetate, methyl cellulose, carboxymethyl cellulose or protamine sulfate. The concentration of macromolecules as well as the methods of incorporation can be adjusted in order to control release. Additionally, the agent can be incorporated into particles of polymeric materials such as polyesters, polyamino acids, hydrogels, poly (lactic acid) or ethylenevinylacetate copolymers. In addition to being incorporated, these agents can also be used to trap the compound in microcapsules.

Accordingly, the pharmaceutical composition of the present invention may be delivered via various routes and to various sites in a mammal body to achieve a particular effect. One skilled in the art will recognize that although more than one route can be used for administration, a particular route can provide a more immediate and more effective reaction than another route. Local or systemic delivery can be accomplished by administration comprising application or instillation of the formulation into body cavities, inhalation or insufflation of an aerosol, or by parenteral introduction, comprising intramuscular, intravenous, peritoneal, subcutaneous, intradermal, as well as topical administration.

The active ingredients of the present invention can be provided in unit dosage form wherein each dosage unit, e.g., a teaspoonful, tablet, solution, or suppository, contains a predetermined amount of the composition, alone or in appropriate combination with other active agents. The term “unit dosage form” as used herein refers to physically discrete units suitable as unitary dosages for human and mammal subjects, each unit containing a predetermined quantity of the compositions of the present invention, alone or in combination with other active agents, calculated in an amount sufficient to produce the desired effect, in association with a pharmaceutically acceptable diluent, carrier, or vehicle, where appropriate. The specifications for the unit dosage forms of the present invention depend on the particular effect to be achieved and the particular pharmacodynamics associated with the pharmaceutical composition in the particular host.

These methods described herein are by no means all-inclusive, and further methods to suit the specific application will be apparent to the ordinary skilled artisan. Moreover, the effective amount of the compositions can be further approximated through analogy to compounds known to exert the desired effect.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the compounds of the present invention and practice the claimed methods. The following working examples therefore, specifically point out the preferred embodiments of the present invention, and are not to be construed as limiting in any way the remainder of the disclosure.

### Example 1

### The Acetylation of Tau Inhibits its Function and Promotes Pathological Tau Aggregation

The microtubule associated protein tau promotes neuronal survival through binding and stabilization of MTs. Phosphorylation regulates tau-microtubule interactions and hyperphosphorylation contributes to the aberrant formation of insoluble tau aggregates in Alzheimer's disease (AD) and related tauopathies (Lee, et al., 2001, Annu Rev. Neurosci. 24:1121-1159). However, other pathogenic post-translational tau modifications have not been well characterized. Here, it is demonstrated that tau acetylation inhibits tau function via impaired tau-microtubule interactions and promotes pathological tau aggregation. Mass spectrometry analysis identified specific lysine residues, including lysine 280 (K280) within the microtubule-binding motif as the major sites of tau acetylation. Immunohistochemical and biochemical studies of brains from tau transgenic mice and patients with AD and related tauopathies showed that acetylated tau pathology is specifically associated with insoluble, Thioflavin-positive tau aggregates. Thus, tau K280 acetylation in the studies presented herein was only detected in diseased tissue, suggesting it may have a role in pathological tau transformation. This study indicates that tau K280 acetylation is a potential target for drug discovery and biomarker development for AD and related tauopathies.

To determine whether tau acetylation is linked to tau aggregation and disease pathogenesis, tau acetylation was assessed in vitro, in cell-based models, in tau Tg mouse models, and in a wide spectrum of human tauopathies including AD. Evidence that tau acetylation at specific lysine residues impairs tau-mediated stabilization of MTs and enhances tau aggregation is provided herein. Furthermore, tau acetylation is associated with insoluble, thioflavin-positive NFTs in tau Tg mouse models and human tauopathies, implicating tau acetylation as a potential factor in the pathogenesis of neurodegenerative tauopathies.

The materials and methods employed in these experiments are now described.

Plasmids and Cell Culture

WT-CBP and CBP-LD inactive mutants as well as WT-HDAC6 and HDAC6-H803A enzyme-inactive mutant expression plasmids were obtained. The longest human tau isoform (designated Tau-T40) was cloned into pCDNA5/TO vector (Invitrogen) and site-directed mutagenesis (Quikchange kit; Stratagene) was used to create K→Q or K→R mutations at residues K163, K280, K281, K369 as indicated. Indicated plasmids were transfected into either QBI-293 cells or stably expressing HEK-T40 cells using Fugene (Roche). HEK-T40 cells were generated using 293 T-Rex cells (Invitrogen) transfected with pcDNA5/TO WT-T40 and selected with 200 μg ml⁻¹ of hygromycin and 5 μg ml⁻¹ blasticidin. Resistant clones were isolated from single cells and screened for tau expression by IF and immunoblotting. IF and MT bundling analysis of transiently transfected QBI-293 cells were performed as previously described (Vogelsberg-Ragaglia, et al., 2000, Mol. Biol. Cell. 11:4093-4104). HDAC6 siRNA (Invitrogen) sequence was as follows: 5′-AAAGUUGGAACUCUCACGGUGCAGC-3′ (SEQ ID NO: 1) and was transfected using RNAi Max reagent (Invitrogen) following manufacturer protocols. Monoclonal antibodies used for western analysis were as follows: T46 (1:1000), T14 (1:1000), PHF-1 (1:1000) and AT-8 (1:500). Polyclonal antibodies used were as follows: ac-K280 (1:1000), anti-acetyl-lysine (1:1000, Cell Signaling), E10 (1:500), HDAC6 (1:1000, Santa Cruz).

Recombinant Tau In Vitro Methods

Tau K18 protein was either mock acetylated or acetylated in vitro using acetyl-CoA substrate as indicated elsewhere herein. Acetylation reaction samples were pooled together, concentrated to 1 ml using Amicon Ultra centrifugal filter units (Millipore Corporation) and ten-fold diluted with 100 mM sodium acetate buffer (pH 7.0) three times followed by concentration to 100 μl of concentrated tau protein. Protein concentrations were determined using the bicinchoninic acid protein assay (Pierce) with bovine serum albumin as the standard. Acetylated K18 and mock-treated K18 were assessed by a light-scattering assay as described elsewhere herein. For fibrillization reactions, 10-20 μM K18 proteins were incubated at 37° C. with equal molar ratio of heparin in 100 mM sodium acetate buffer (pH 7.0) containing 2 mM dithiothreitol (DTT). The extent of fibrillization was assessed at different time points by ThT fluorescence assay, sedimentation analysis and negative staining EM as described elsewhere herein. For ThT fluorescence assay, ThT diluted in 100 mM glycine buffer (pH 8.5) was added and fluorometric readings were taken at 450 nm (excitation) and 510 nm (emission). The assay was performed in triplicate. For sedimentation test, 40 μl fibrillization mixtures were centrifuged at 100,000 g for 30 min at 4° C. to separate fibrils soluble supernatant fractions. Pellets were washed once with 100 mM sodium acetate buffer (pH 7.0) and resuspended in 40 μl of the same buffer. Equal volume of fractions were analysed by 15% SDS-PAGE gels followed by Coomassie staining For negative staining EM, 6 μl of fibrillization mixture from each time point were placed on 300 meshed Formvar/carbon film-coated copper grids (Electron Microscopy Sciences) for 3 min, washed twice with 50 mM Tris buffer (pH 7.6) for 5 min each, stained with 10-15 drops of 2% uranyl acetate and visualized with Jeol 1010 transmission electron microscope (Jeol).

Peptide and Antibody Generation

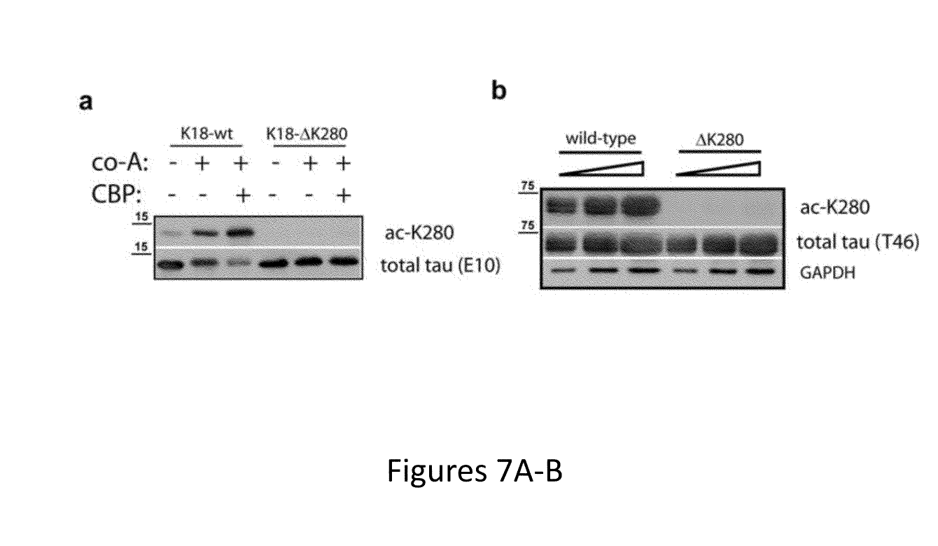

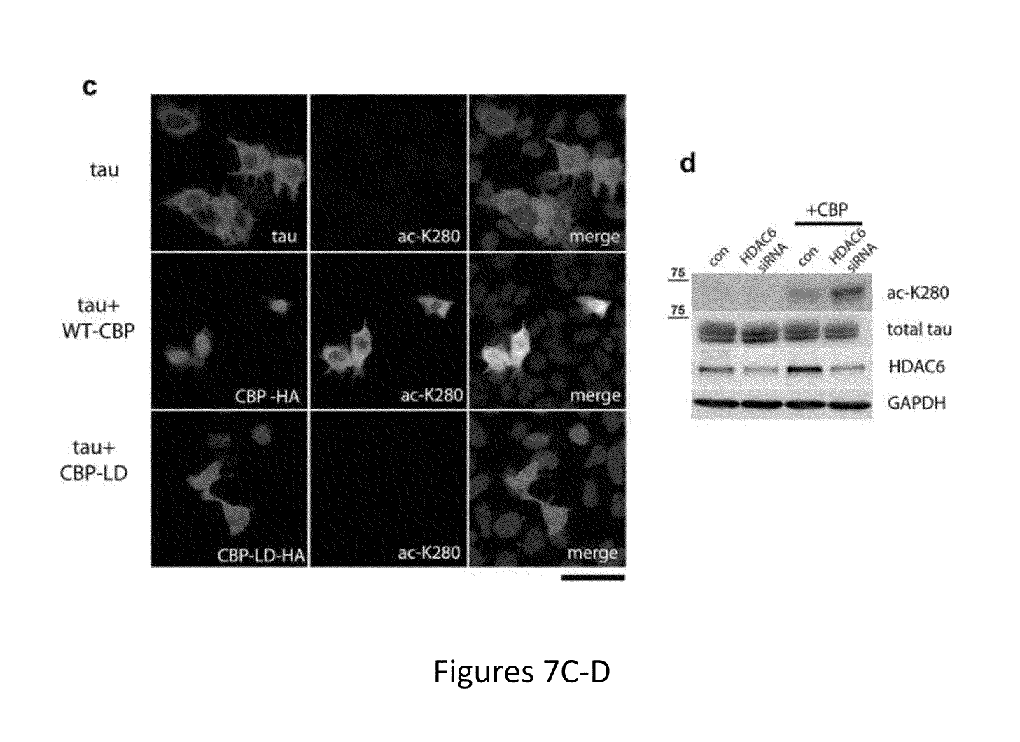

Polyclonal anti-acetyl tau K280 antibodies were generated using the acetylated tau peptide C-QIINKKLDLSN (SEQ ID NO. 2) containing K280 acetylation to immunize rabbits (Pocono Rabbit Farm and Laboratory). Double affinity purification was performed using native and acetylated peptides sequentially using Sulfolink columns (Pierce Biotechnology). Site specificity of ac-K280 was confirmed in vitro and in cells using tau proteins lacking residue K280 (FIG. 7).

Histology and IHC

Tissue preparation and IHC were performed on monogenic PS19 and bigenic PS19; PDAPP transgenic mice at 4, 8 and 11 months of age as described. Mice were anaesthetized and transcardially perfused with phosphate buffer saline, pH 7.0 in accordance with protocols approved by the Institutional Animal Care and Use Committee of the University of Pennsylvania. Brains were surgically removed and fixed in 4% neutral-buffered formalin. Coronal slabs were fixed in neutral-buffered formalin and paraffin embedded. Paraffin blocks were sectioned at 6 μm through the entire block. Tissues sections were stained using a Polymer horseradish peroxidase detection system (Biogenex) and automatically stained using the i6000 Automated Staining System (Biogenex). For human studies, fixed, paraffin-embedded tissue blocks were obtained from the Center for Neurodegenerative Disease Research Brain Bank at the University of Pennsylvania. Consent for autopsy was obtained from legal representatives for all subjects in accordance with local institutional review board requirements. Briefly, fresh tissues from each brain were fixed with 70% ethanol in 150 mmol l⁻¹ NaCl, infiltrated with paraffin and cut into 6-μm serial sections. IHC was performed using the avidin-biotin complex detection system (Vector Laboratories) and 3,3′-diaminobenzidine. Briefly, sections were deparaffinized and rehydrated, antigen retrieval was done by incubating section in 88% formic acid, endogenous peroxidases were quenched with 5% H₂O₂ in methanol for 30 min and sections were blocked in 0.1 mol l⁻¹ Tris with 2% fetal bovine serum for 5 min. Primary antibodies were incubated for either 1 or 2 h at room temperature or overnight at 4° C. The following primary antibodies were used for IHC analysis: affinity purified rabbit anti-acetyl-tau (ac-K280; 1:250-1:2000), mouse anti-phospho-tau Ser202/Thr205 (ATB; 1:15,000), mouse anti-phospho-tau Ser396/Ser404 (PHF-1; 1:1000). After washing, sections were sequentially incubated with biotinylated secondary antibodies for 1 h and avidin-biotin complex for 1 h. Bound antibody complexes were visualized by incubating sections in a solution containing 100 mM TrisHCl, pH 7.6, 0.1% Triton X-100, 1.4 mM diaminobenzidine, 10 mM imidazole and 8.8 mM H₂O₂. Sections were then lightly counterstained with hematoxylin, dehydrated and coverslipped.

Double-Labelling IF

Double-labelling IF analyses were performed using Alexa Fluor 488- and 594-conjugated secondary antibodies (Molecular Probes), treated for autofluorescence with Sudan Black solution and coverslipped with Vectashield mounting medium (Vector Laboratories). Double-labelling ThS and IF was performed through sequential methods initially staining sections with ThS followed by IF. Briefly, sections were deparaffinized and hydrated to dH₂O, immersed in PBS pH7.3 for 5 min followed by immersion in 0.05% KMnO₄/PBS for 20 min. Sections were rinsed in PBS and destained in 0.2% K₂S₂O₅/0.2% oxalic acid/PBS and immersed in 0.0125% ThS/40% EtOH/60% PBS and differentiated in 50% EtOH/50% PBS for 10-15 min. After differentiation, sections were incubated with rabbit anti-ac-K280 overnight at 4° C. using standard IF techniques. Digital images were obtained using an Olympus BX 51 microscope (Olympus) equipped with bright-field and fluorescence light sources using a ProgRes C14 digital camera (Jenoptik AG) and Adobe Photoshop, version 9.0 (Adobe Systems) or digital camera DP71 (Olympus) and DP manager (Olympus).

Biochemical Extraction

PHFs were isolated from normal and tauopathy brains as previously described (Lee, et al., 1999, Methods Enzymol. 309:81-89). Briefly, grey matter was homogenized in 3 vol per g of cold High Salt RAB buffer (0.75 M NaCl, 100 mM Tris, 1 mM EGTA, 0.5 mM MgSO₄, 0.02 M NaF, 2 mM DTT, pH 7.4). All buffers were supplemented with protease inhibitor cocktail, phosphatase inhibitor and HDAC inhibitors (2 μM TSA and 10 mM nicotinamide). Homogenates were incubated at 4° C. for 20 min to depolymerize MTs, then, centrifuged at 100,000 g for 30 min at 4° C. Pellets were re-homogenized and centrifuged in 3 vol per g of cold High Salt RAB buffer. Resultant pellets were homogenized in 5 vol per g of cold PHF extraction buffer (10 mM Tris pH 7.4, 10% sucrose, 0.85 M NaCl, 1 mM EGTA, pH 7.4) and centrifuged at 15,000 g for 20 min at 4° C. Sarkosyl was added to the supernatant (1% final concentration) and incubated at 4° C. overnight followed by centrifugation for 30 min at 100,000 g to generate crude PHF-tau fractions, which were solubilized by formic acid extraction. Fractionation of mouse cortices was performed by sequential extraction using buffers of increasing strength. Brain tissue was homogenized in 5 vol per g of low-salt buffer (10 mM Tris pH 8.0, 2 mM EDTA, 1 mM DTT) and centrifuged at 100,000 g for 30 min to generate low-salt fractions. Myelin floatation was performed on pellets re-extracted in low-salt buffer supplemented with 20% sucrose. Resulting pellets were re-extracted in 5 vol per g of RIPA buffer (50 mM Tris pH 8.0, 150 mM NaCl, 1% NP-40, 5 mM EDTA, 0.5% sodium deoxycholate, 0.1% SDS). Finally, resultant insoluble pellets were extracted in 1 vol per g of tissue in urea buffer (7 M urea, 2 M Thiourea, 4% CHAPS, 30 mM Tris, pH 8.5). Soluble and insoluble fractions were analysed by SDS-PAGE electrophoresis and western blotting using the indicated antibodies to detect total, acetylated or phosphorylated tau proteins.

The results of the experiments are now described.

### Acetylation Impairs Tau Function and Promotes Tau Fibrillization

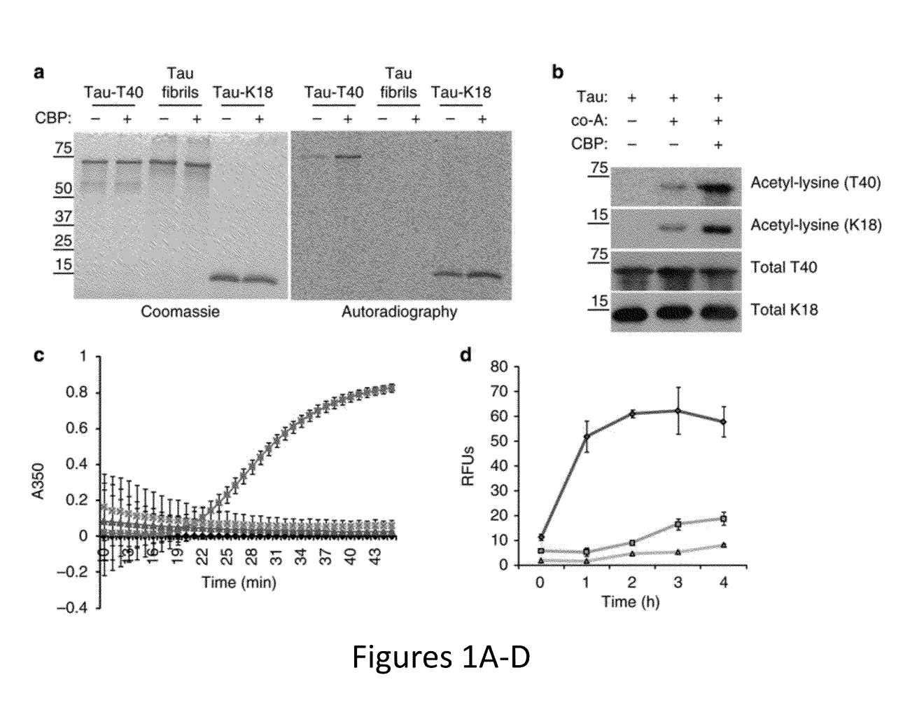

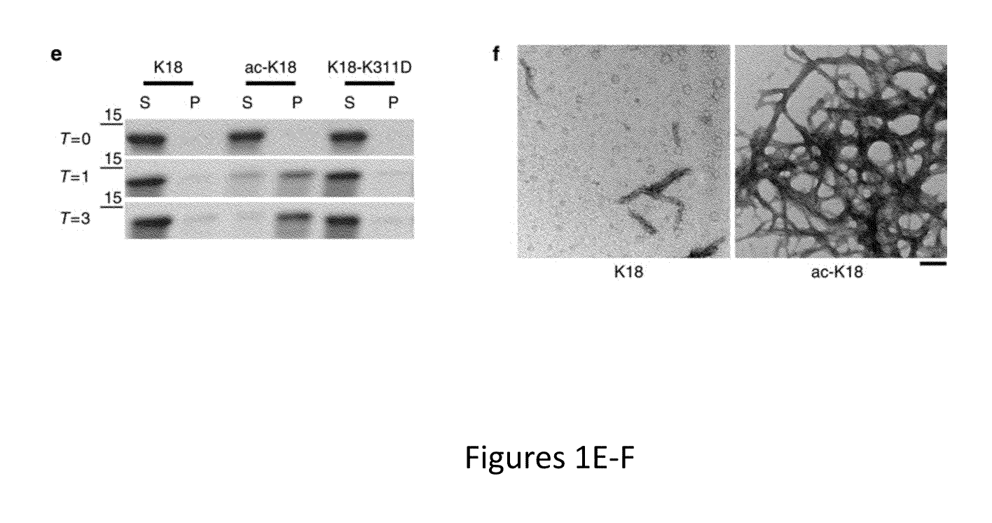

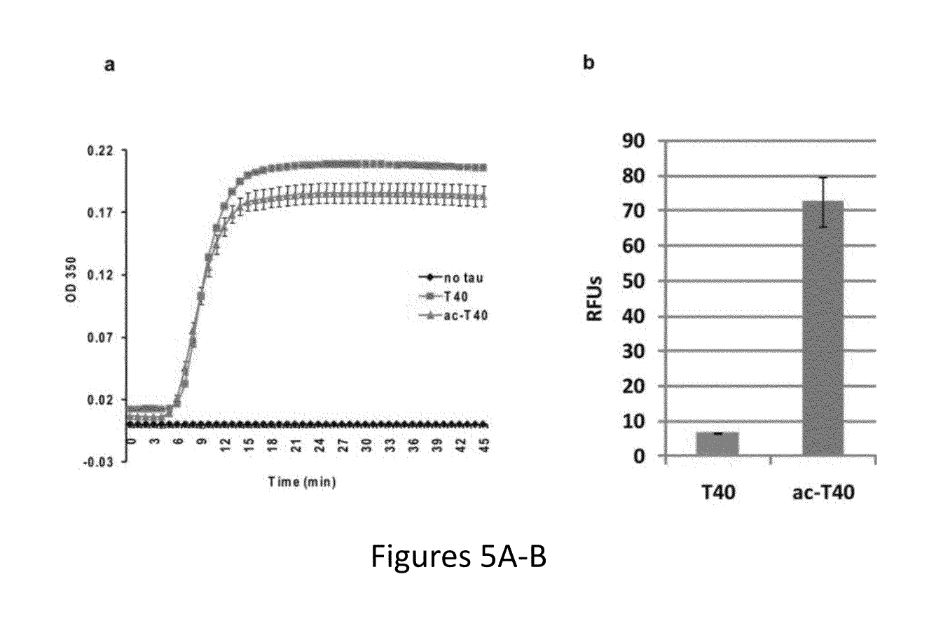

To fully characterize tau acetylation, recombinant full-length tau (T40) or a fragment encompassing all four MT-binding repeats (K18), were incubated with [¹⁴C]-acetyl Co-A in the presence of the acetyltransferase Creb-binding protein (CBP) (Chan & La Thangue, 2001, J. Cell Sci. 114: 2363-2373). Although tau-K18 and tau-T40 displayed low-level auto-acetylation frequently seen with high-affinity substrates, CBP enhanced the acetylation of both full-length T40 and the K18 fragment, suggesting acetylated lysine residues lie within the MT-binding region (FIG. 1A). Tau-T40 was fibrillized in vitro to generate tau fibrils in which the MT-binding region forms the fibril core. These tau fibrils could not be acetylated indicating lysine residues are not accessible to acetylation in fibrillized tau. To confirm tau acetylation in vitro, immunoblotting using a pan-anti-acetyl-lysine antibody revealed that both T40 and K18 were acetylated in the presence of CBP (FIG. 1B). To assess the significance of tau acetylation, the ability of acetylated tau-K18 (FIG. 1C) or full-length tau-T40 (FIG. 5) proteins to promote MT assembly was determined using a light-scattering technique (Hong, et al., 1998, Science 282:1914-1917). As expected, native K18 promoted tubulin assembly into MTs as determined by absorbance at 350 nm. In contrast, acetylated K18 failed to promote MT assembly, similar to the negative control K18 containing the K311D mutation, previously shown to impair tau-MT interactions (Crowe, et al., 2007, Biochem. Biophys. Res. Commun. 358:1-6; Li & Lee, 2006, Biochemistry 45:15692-15701).

In addition to promoting MT assembly, the MT repeat domain is the core region required for tau fibrillization into AD paired helical filaments (PHFs) and straight filaments found in other tauopathies (Goedert, et al., 1996, Nature 383:550-553; Perez, et al., 1996, J. Neurochem. 67:1183-1190). Given the robust tau acetylation within the MT-binding region, it was determined whether acetylation of tau-K18 (FIG. 1D) or tau-T40 (FIG. 5) affects tau fibrillization. Using a heparin-induced tau fibrillization assay quantified by Thioflavin-T (ThT) binding, native K18 did not fibrillize during the 4-h incubation period, whereas acetylated K18 fibrillized rapidly by 3 h indicating acetylation enhanced the K18 fibrillization rate (FIG. 1D). Similar enhancement of tau-T40 aggregation was also observed (FIG. 5). Sedimentation analysis of acetylated K18 confirmed this finding and showed significant fibril accumulation in pellet fractions from 1 to 3 h, whereas native K18 was predominantly in the supernatant fraction (FIG. 1E). As expected, the negative control K18-K311D mutant did not fibrillize using both ThT and sedimentation assays. Confirming these observations, negative staining electron microscopy (EM) showed that acetylated K18 but not native K18 formed abundant filaments after 4 h (FIG. 1F). Thus, acetylation of tau proteins in vitro not only impaired the ability of tau to promote MT assembly but also enhanced tau fibrillization, similar to tau harboring MT-binding region mutations in several FTDP-17 kindreds (Barghorn, et al., 2000, Biochemistry 39:11714-11721; von Bergen, et al., 2001, J. Biol. Chem. 276:48165-48174).

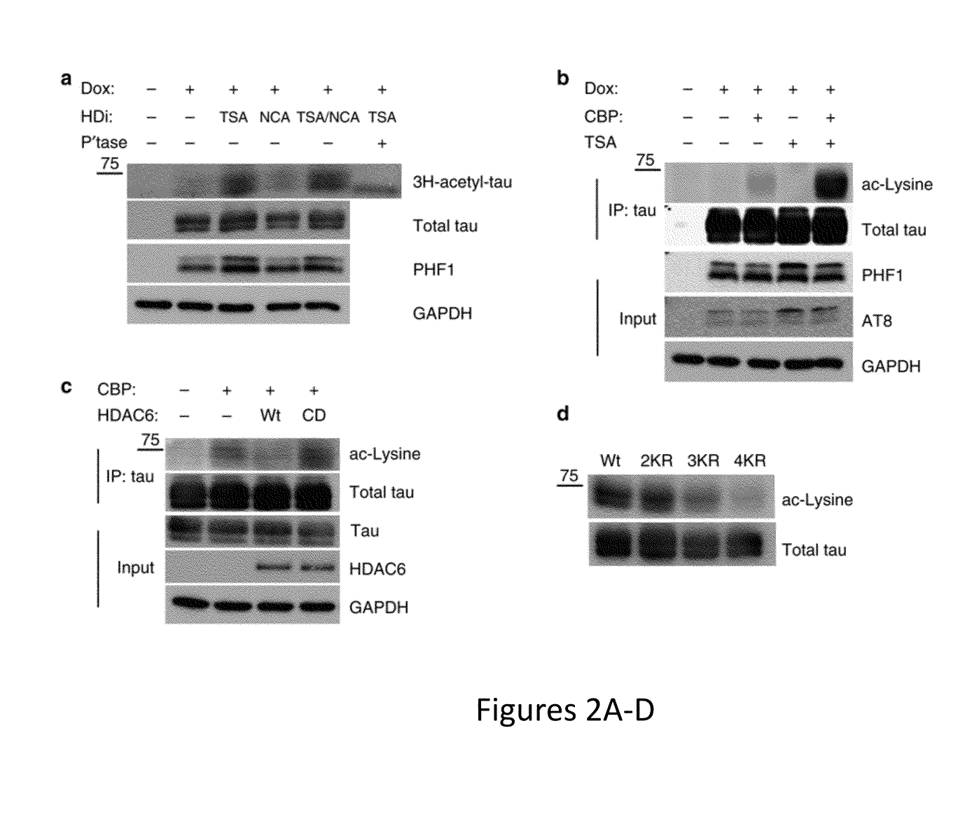

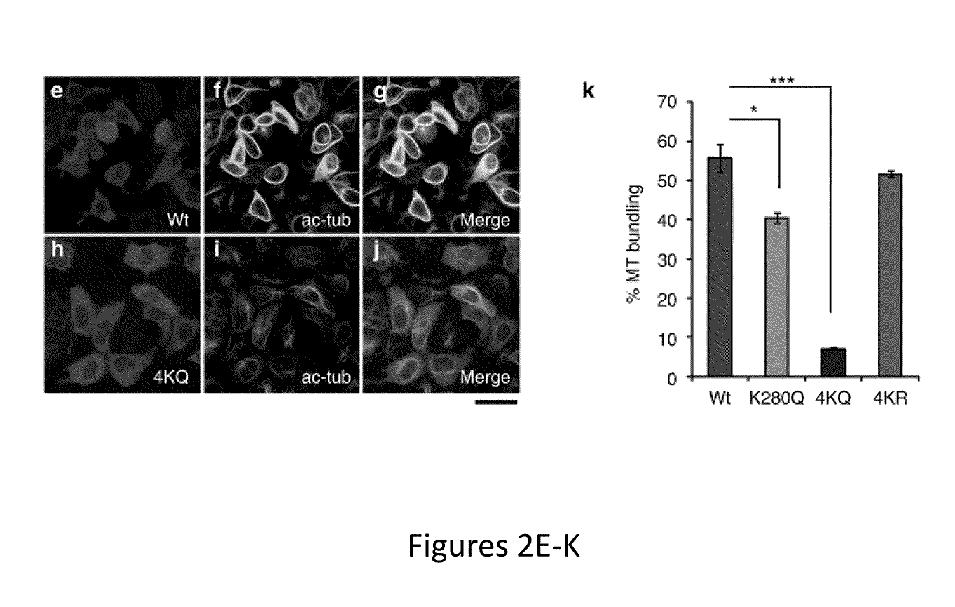

To characterize tau acetylation in cells, doxycycline-inducible HEK-293 cells stably expressing full-length tau-T40 (HEK-T40 cells) were labeled with [³H]-acetate followed by immunoprecipitation, SDS-PAGE and autoradiography (FIG. 2A). Although low-level tau acetylation was observed in untreated HEK-T40 cells, treatment with the pan histone deacetylase (HDAC) inhibitor trichostatin A (TSA), but not the Sir2 class inhibitor nicotinamide (Haigis & Sinclair, 2010, Annu Rev. Pathol. 5:253-295), resulted in a dramatic increase in acetylated tau levels. To further confirm tau acetylation, tau was immunoprecipitated from HEK-T40 cells transiently transfected with CBP, and acetylated tau was detected using an anti-acetyl-lysine antibody (FIG. 2b). CBP transfection alone promoted detectable tau acetylation, however, CBP expression in combination with TSA treatment led to a dramatic accumulation of acetylated tau. Notably, acetylated tau can be modified by HDAC activity as co-expression of CBP with wild-type (WT) HDAC6, but not a catalytically dead HDAC6 mutant, resulted in tau deacetylation (FIG. 2C). Thus, tau is dynamically regulated by reversible acetylation and deacetylation reactions.

### Identification and Characterization of Acetylated Lysine Residues in Tau

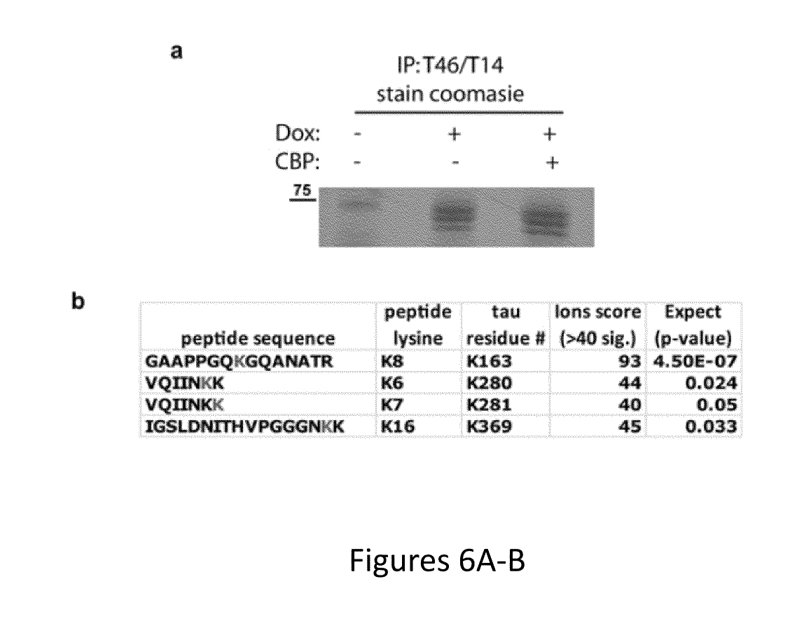

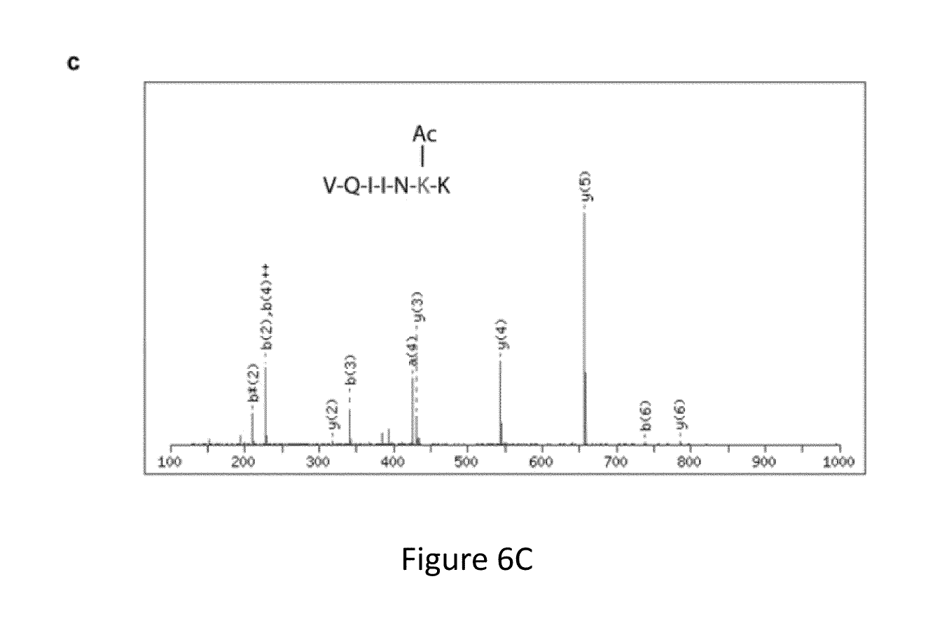

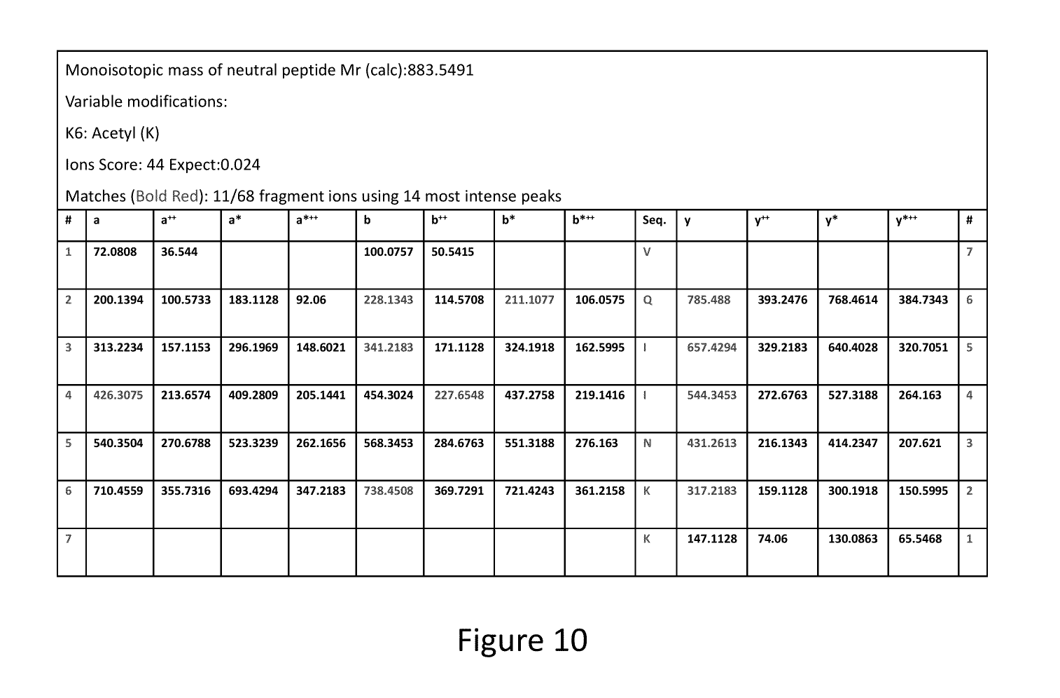

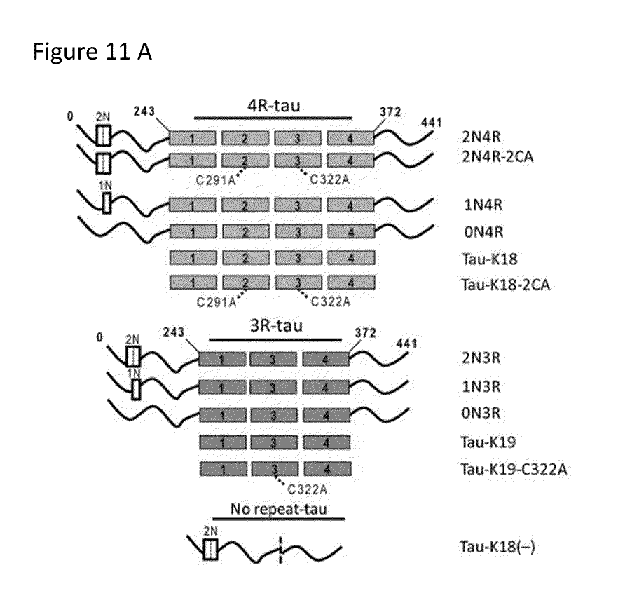

To identify acetylated lysine residues in tau, mass spectrometry analysis using nanoLC/nanospray/MS/MS was performed on recombinant T40 acetylated in vitro. Multiple lysine residues within the repeat region and adjacent proline-rich region showed significant acetylation (Table 3, P value <0.05 significance score). To validate in vitro acetylated sites, CBP-transfected HEK-T40 cells were treated with TSA and immunoprecipitated tau protein bands were excised for mass spectrometry analysis (FIG. 6 and FIG. 10). Significant acetylation was detected on the double lysine K280/K281 within the peptide V-Q-I-I-N-K-K (SEQ ID NO. 3) in the second MT-binding repeat as well as K163 and K369 within the proline-rich region and MT-binding region, respectively (see Table 1). Western analysis of lysine→arginine mutants demonstrated that mutation of all four lysine residues (tau-4KR) dramatically reduced tau acetylation in cells, indicating that K163/280/281/369 represent the major sites of tau acetylation (FIG. 2D).

To evaluate the functional significance of tau acetylation, K→Q acetylation mimetic mutants were generated and expressed in HEK-293 cells. Consistent with previous reports (Vogelsberg-Ragaglia, et al., 2000, Mol. Biol. Cell. 11:4093-4104), WT tau overexpression stabilized MTs and promotes MT bundling, as detected by immunofluorescence (IF) using an anti-acetyl-tubulin antibody (FIG. 2E through FIG. 2G). However, expression of a tau-4KQ acetylation mimetic (K163/280/281/369Q), but not a tau-4KR non-mimetic mutant, showed a dramatic reduction in MT bundling activity (WT ˜55% versus 4KQ ˜7% versus 4KR ˜50% bundling), consistent with loss of tau function induced by acetylation (FIG. 2H through FIG. 2J). Interestingly, MT bundling observed with the single K280Q mimetic was significantly reduced (˜40% bundling), indicating acetylation of a single-lysine residue, that is, K280, is critical in regulating tau function (FIG. 2K). These results suggest that tau acetylation inhibits tau-mediated stabilization of MTs.

TABLE 1
Tau acetylation sites identified by
mass spectrometry.

| | | |
|---|---|---|
| Lysine | Peptide | P value |

Acetylated lysine residues are highlighted
in bold text with corresponding P values.

| | | | |
|---|---|---|---|
| K163 | GAAPPGQKGQANATR | (SEQ ID NO. 4) | 4.50E− |
| | | | 0.7 |
| K280 | VQIINKK | (SEQ ID NO. 3) | 0.024 |
| K281 | VQIINKK | (SEQ ID NO. 3) | 0.05 |
| K369 | IGSLDNITHVPGGGNKK | (SEQ ID NO. 5) | 0.033 |

### Tau Acetylation on K280 Detects Tau Pathology in Tg Mouse Models

Genetic studies have shown that deletion of K280 (ΔK280) is associated with FTDP-17 familial tauopathy (Rizzu, et al., 1999, Am. J. Hum. Genet. 64:414-421). Although the disease mechanism associated with ΔK280 is not fully understood, in vitro analysis of ΔK280 demonstrated impaired MT assembly (Goode & Feinstein, 1994, J. Cell Biol. 124:769-782) and enhanced PHF aggregation (von Bergen, et al., 2001, J. Biol. Chem. 276:48165-48174; Mukrasch, et al., 2005, J. Biol. Chem. 280:24978-24986). Thus, K280 acetylation functionally mimics ΔK280, potentially by inhibition of electrostatic tau-MT interactions. To monitor K280 acetylation in brain tissue, a polyclonal antibody was generated against an acetylated K280 tau peptide. Double affinity purification yielded a site-specific acetylated K280 antibody (hereafter referred to as anti-ac-K280) that detected acetylated WT tau, but not a ΔK280 mutant tau demonstrated by in vitro and cell-based acetylation assays (FIG. 7). Ac-K280 positive cells were detected in the presence of active CBP, but not a catalytically inactive CBP mutant (CBP-LD). Moreover, expression of an HDAC6 small interfering RNA led to increased levels of ac-K280, further confirming the specificity of the anti-ac-K280 antibody and providing additional evidence that HDAC6 is a tau deacetylase (FIG. 7).

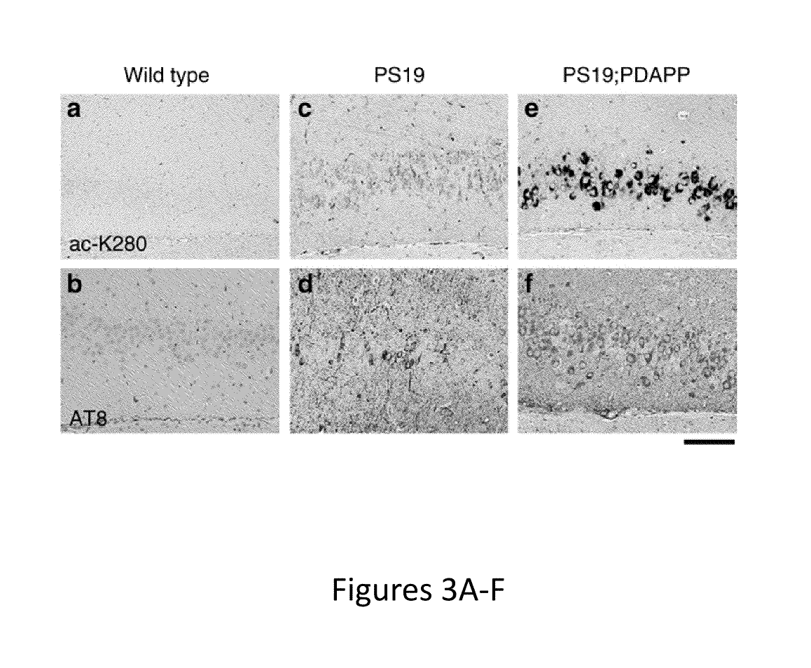

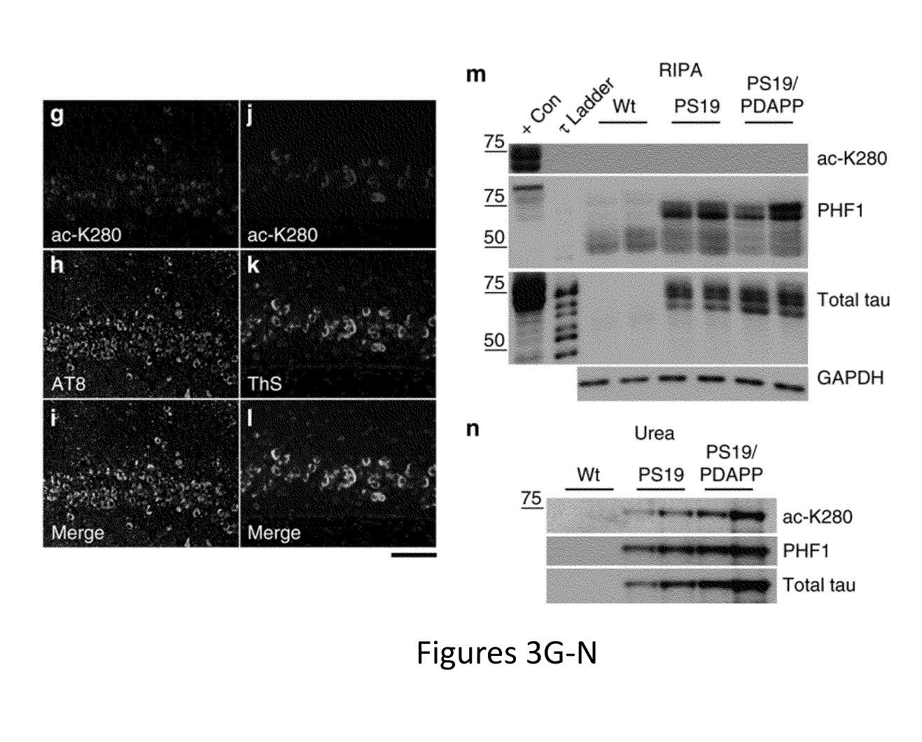

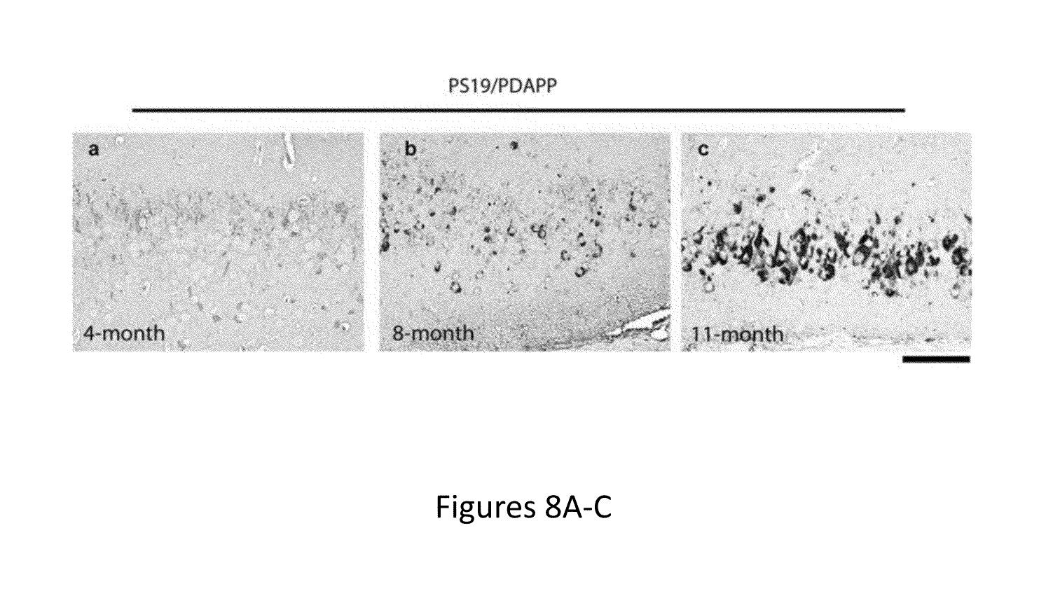

Using anti-ac-K280, tau acetylation in mouse brain was probed by immunohistochemistry (IHC) using two tau Tg mouse models that recapitulate tau pathology and NFT formation: PS19 monogenic mice expressing P301S mutant tau (Yoshiyama, et al., 2007, Neuron 53:337-351) and PS19; PDAPP bigenic mice, which additionally express the APP V717F mutant transgene that accelerates NFT formation and enhances tau amyloidogenesis (Hurtado, et al., 2010, Am. J. Pathol. 177:1977-1988). Monogenic PS19 tau Tg mice revealed moderate neuronal ac-K280 immunoreactivity in both cortex and hippocampus when compared with non-Tg mice, although abundant hyperphosphorylated tau was detected by AT8, a phospho-tau specific antibody (FIG. 3A through FIG. 3D). However, bigenic PS19; PDAPP mice showed profound age-dependent increase in ac-K280 tau pathology (see FIG. 8), particularly in the hippocampus, in which the intensity of ac-K280 staining was increased relative to monogenic PS19 mice (FIG. 3E and FIG. 3F). Moreover, significant co-localization of ac-K280 immunoreactivity was observed with AT8 in tau inclusions of PS19; PDAPP bigenic mice using double-labeling IF (FIG. 3G through FIG. 3I). Ac-K280-positive tau inclusions were also detected by Thioflavin-S (ThS), which demonstrates that tau amyloid lesions are prominently acetylated in these mice (FIG. 3J through FIG. 3L).

To determine the biochemical properties of acetylated tau, sequential extraction of normal and pathological tau from WT, PS19 and PS19/PDAPP mouse cortices was conducted using buffers of increasing strength. RIPA and urea solubilized brain extracts from mouse cortices were analyzed by western blotting using anti-ac-K280, PHF-1 and total tau (T14/T46) antibodies. RIPA extracted cortical lysates contained the majority of the soluble tau pool, indicated by total tau levels (FIG. 3M). In contrast, anti-ac-K280 reactivity was undetectable in RIPA-solubilized extracts, but instead accumulated in the insoluble urea fraction (FIG. 3N). Consistent with the IHC analysis, immunoblotting also revealed increased ac-K280 levels in PS19; PDAPP bigenic compared with PS19 monogenic mice. Thus, tau acetylation is associated with biochemically insoluble, ThS-positive tau aggregates in tau Tg mouse models.

### Tau Acetylation on K280 is Found in a Wide Range of Human Tauopathies

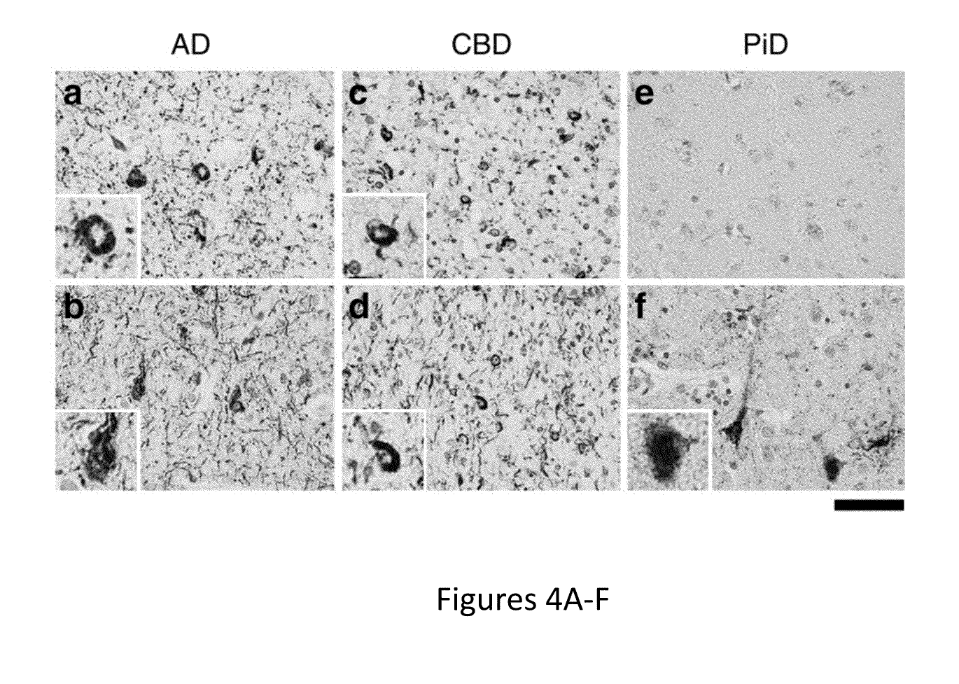

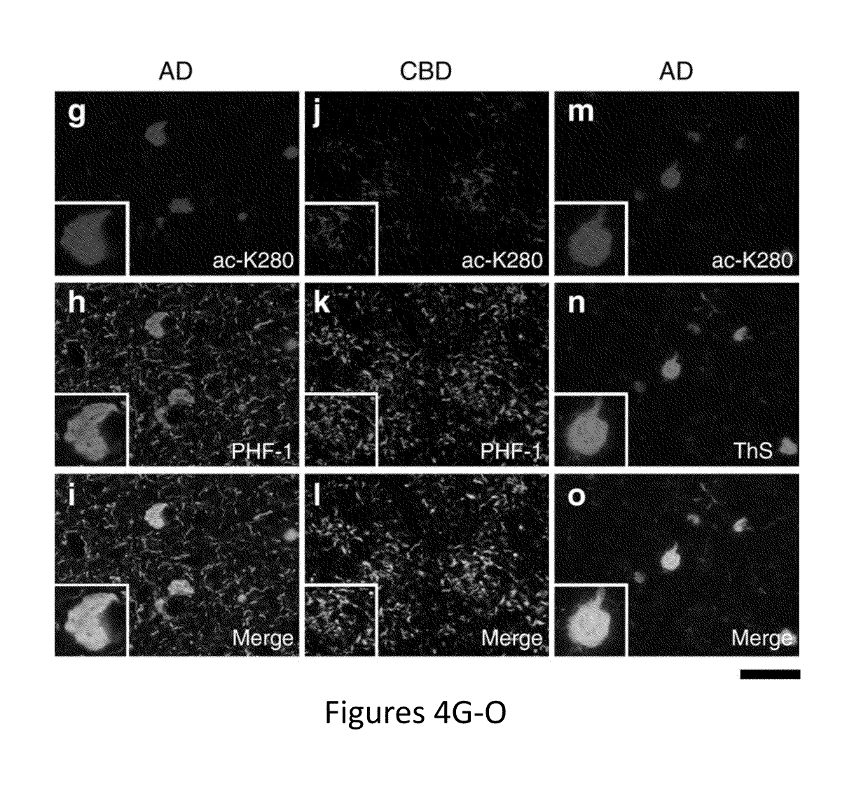

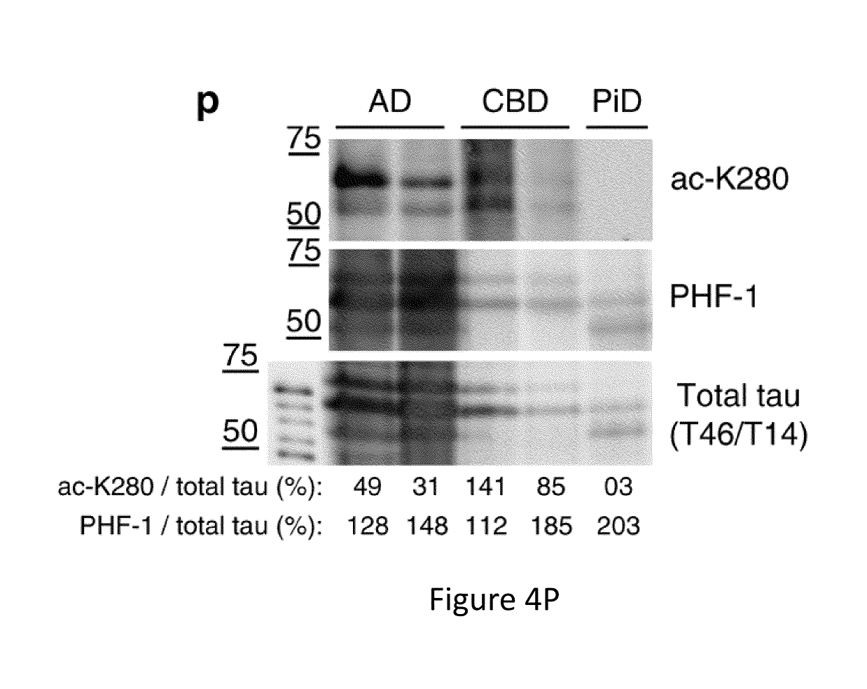

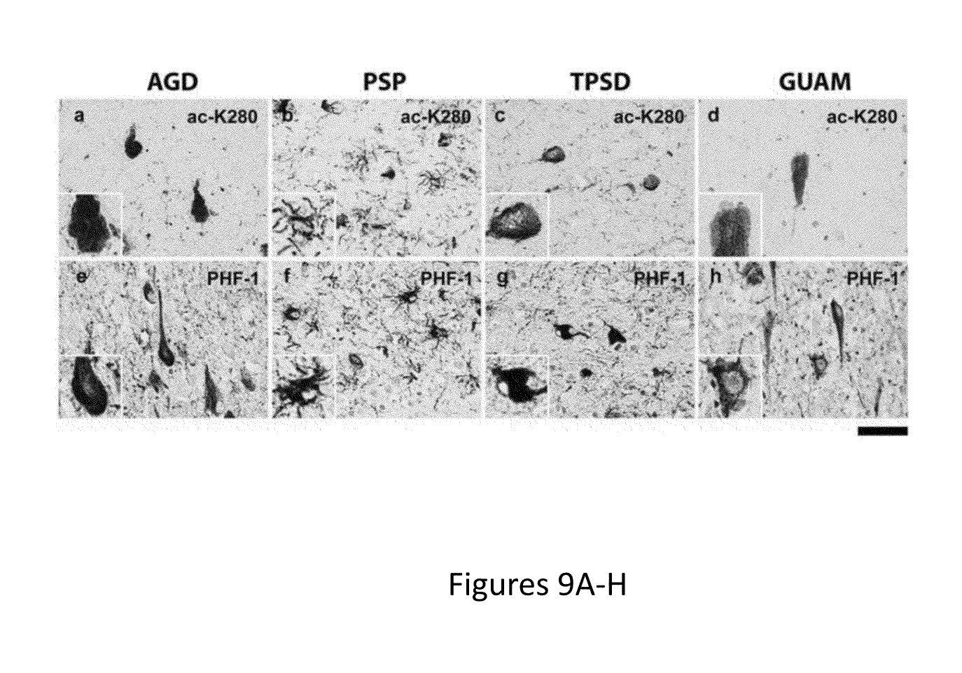

To investigate tau K280 acetylation in human neurodegenerative tauopathies, IHC was conducted on AD, corticobasal degeneration (CBD) and Pick's disease (PiD) cases (see Table 2 for human cases used in this study). These tauopathies share abundant filamentous tau pathology in affected brain regions, but differ in their patterns of insoluble tau isoforms with equal ratios of 3R/4R tau in AD, predominantly 4R-tau in CBD, and predominantly 3R-tau in PiD. Cortical brain sections of tauopathies were analysed using anti-ac-K280, which recognizes only 4R-tau isoforms as 3R-tau lacks K280 because of exon 10 alternative mRNA splicing. Remarkably, anti-ac-K280-positive NFTs and neuropil threads were prominently observed in AD cortex and resemble pathology detected by PHF-1 (FIG. 4A and FIG. 4B). CBD has extensive grey and white matter tau pathology of neuronal and glial tau inclusions, and anti-ac-K280 immunostained these tau pathologies similar to PHF-1 (FIG. 4C and FIG. 4D). Importantly, analysis of PiD inclusions containing primarily 3R-tau isoforms revealed no detectable anti-ac-K280 immunoreactivity (FIG. 4E and FIG. 4F), further validating anti-ac-K280 specificity. To determine whether acetylated tau is present within tau tangles, double IF microscopy revealed substantial co-localization of anti-ac-K280 and PHF-1 in all 4R-tau-specific tauopathies, including AD and CBD (FIG. 4G through FIG. 4I, and FIG. 4J through FIG. 4L, respectively). Moreover, ThS-positive NFTs present in AD brains also co-localized with anti-ac-K280 immunoreactive tau inclusions (FIG. 4M through FIG. 4O). To determine the scope of acetylated tau lesions in human disease, these observations were extended to a wide spectrum of tauopathies with 4R-tau pathology (see case demographics in Table 2). Indeed, anti-ac-K280-positive tau aggregates were observed in all other tauopathies examined including progressive supranuclear palsy, argyrophilic grain disease, tangle predominant senile dementia and Guam parkinsonism-dementia complex (FIG. 9). Thus, K280 acetylation is a feature found in a variety of human 4R or 3R/4R tauopathies including AD, but not 3R-tauopathies such as PiD.

To demonstrate acetylated tau is present in PHFs of human tauopathies, PHFs were extracted from cortical regions of AD, CBD and PiD brains (Bramblett, et al., 1993, Neuron 10:1089-1099; Hong, et al., 1998, Science 282:1914-1917) and immunoblotted using anti-ac-K280, PHF-1 and T14/T46 (total tau) antibodies (FIG. 4P). Prominent PHF-tau isoforms were detected with PHF-1 and T14/T46 antibodies, confirming isolation of insoluble and hyperphosphorylated 4R/3R-tau (AD), 4R-tau (CBD) or 3R-tau (PiD) species. Consistent with the above IHC results, anti-ac-K280 detected 4R tau isoforms in AD and CBD cases, but not the 3R-tau isoforms in PiD. Furthermore, acetylation of normal soluble tau was never observed. These results are consistent with more prominent acetylation occurring specifically on pathologically insoluble, aggregated PHF-tau species.

TABLE 2
Demographics of human tauopathy subjects used in this study.

| | | | Age | | | | | Ac- |
|---|---|---|---|---|---|---|---|---|
| Case | | | at | Year | Duration | PMI | | K280 |
| no. | Diagnosis | Sex | death | onset | (years) | (h) | Dementia | positive |
| 1 | Normal control | Female | 65 | NA | NA | 19 | No | − |
| 2 | Normal control | Male | 74 | NA | NA | 7.5 | No | − |
| 3 | Alzheimer's | Female | 90 | 1998 | 3 | 9 | Yes | +++ |
| | disease | | | | | | | |
| 4 | Alzheimer's | Female | 83 | 1997 | 6 | 4 | Yes | +++ |
| | disease | | | | | | | |
| 5 | Alzheimer's | Male | 73 | 1999 | 9 | 9 | Yes | ++ |
| | disease | | | | | | | |
| 6 | Alzheimer's | Male | 66 | 2000 | 8 | 4 | Yes | +++ |
| | disease | | | | | | | |
| 7 | Alzheimer's | Female | 80 | 1997 | 12 | 6 | Yes | ++ |
| | disease | | | | | | | |
| 8 | Argyrophilic | Male | 84 | 1984 | 13 | 13 | Yes | +++ |
| | grain disease | | | | | | | |
| 9 | Argyrophilic | Male | 74 | 1986 | 19 | 19 | Yes | + |
| | grain disease | | | | | | | |
| 10 | Corticobasal | Male | 56 | 2000 | 6 | 15 | NA | +++ |
| | degeneration | | | | | | | |
| 11 | Corticobasal | Male | 86 | 2000 | 6 | 11 | Yes | ++ |
| | degeneration | | | | | | | |
| 12 | Corticobasal | Male | 75 | 2002 | 4 | 12 | No | + |
| | degeneration | | | | | | | |
| 13 | Pick's disease | Female | 62 | NA | NA | 5.5 | Yes | − |
| 14 | Pick's disease | Male | 57 | 1998 | 9 | 11 | Yes | − |
| 15 | Pick's disease | Female | 84 | 1995 | 14 | 24 | Yes | − |
| 16 | Progressive | Female | 71 | 1995 | 10 | 14 | No | + |
| | supranuclear | | | | | | | |
| | palsy | | | | | | | |
| 17 | Progressive | Female | 63 | 2002 | 5 | 6.5 | No | +++ |
| | supranuclear | | | | | | | |
| | palsy | | | | | | | |
| 18 | Progressive | Male | 80 | 2002 | 5 | 19 | NA | + |
| | supranuclear | | | | | | | |
| | palsy | | | | | | | |
| 19 | Tangle | Female | 92 | 1982 | 14 | 12 | Yes | +++ |
| | predominant | | | | | | | |
| | senile dementia | | | | | | | |
| 20 | Tangle | Male | 79 | 1988 | 8 | 16 | Yes | +++ |
| | predominant | | | | | | | |
| | senile dementia | | | | | | | |
| 21 | Tangle | Male | 76 | 1994 | NA | 19 | Yes | + |
| | predominant | | | | | | | |
| | senile dementia | | | | | | | |
| 22 | Guam | Female | 47 | NA | 10 | NA | NA | + |
| | parkinsonism- | | | | | | | |
| | dementia | | | | | | | |
| | complex | | | | | | | |

Abbreviations:
duration, duration of disease;
NA, not applicable;
PMI, postmortem intervals;
(−), no ac-K280 pathology;
(+), rare occurrence of ac-K280 pathology;
(++), moderate occurrence of ac-K280 pathology;
(+++) frequent occurrence of ac-K280 pathology.
Cases listed here were used for both IHC and biochemical analyses.

TABLE 3
Mass spectrometry analysis identified tau acetylation
sites from in vitro acetylated tau-T40.
Recombinant CBP protein was incubated with 1 μg T40 and
0.4 mM acetyl-CoA in 30 μl of reaction buffer (50 mM
Tris-HCl pH 8.0, 10% glycerol, 1 mM DTT, 100 mM EDTA,
1 mM phenylmethylsulfonyl fluoride) for 1 hr at 37° C.
Acetylation was analyzed by SDS-PAGE and Coomassie
staining followed by gel excision for mass spectrometry
using LTQ XL* Linear Ion Trap Mass Spectrometer (Thermo
Scientific). Data was acquired using Xcalibur software
(Thermo Scientific) and analyzed using Mascot software
(Matrix Science).

| | peptide | tau | Ions score | Expect |
|---|---|---|---|---|
| peptide sequence | lysine | residue# | (>40 sig.) | (p-value) |
| TKIATPR | K2 | K150 | 34 | 0.12 |
| (SEQ ID NO: 6) | | | | |
| GAAPPGQKGQANATR | K8 | K163 | 106 | 5.30 E−09 |
| (SEQ ID NO. 4) | | | | |
| IPAKTPPAPK | K4 | K174 | 47 | 0.0049 |
| (SEQ ID NO. 7) | | | | |
| TPPKSPSSAK | K4 | K234 | 46 | 0.0066 |
| (SEQ ID NO. 8) | | | | |
| TPPKSPSSAKSR | K10 | K240 | 47 | 0.0047 |
| (SEQ ID NO. 9) | | | | |
| SKIGSTENLK | K2 | K259 | 44 | 0.011 |
| (SEQ ID NO. 10) | | | | |
| HQPGGGKVQIINK | K7 | K274 | 76 | 6.20 E−06 |
| (SEQ ID NO. 11) | | | | |
| VQIINKK | K6 | K280 | 46 | 0.0059 |
| (SEQ ID NO. 3) | | | | |
| VQIINKK | K7 | K281 | 44 | 0.0099 |
| (SEQ ID NO. 3) | | | | |
| KLDLSNVQSK | K10 | K290 | 61 | 0.00021 |
| (SEQ ID NO. 12) | | | | |
| HVPGGGSVQIVYKPVDLSK | K13 | K311 | 58 | 0.00029 |
| (SEQ ID NO. 13) | | | | |
| IGSLDNITHVPGGGNKK | K16 | K369 | 73 | 1.00 E−05 |
| (SEQ ID NO. 5) | | | | |
| TDHGAEIVYKSPVVSGDTSPR | K10 | K395 | 56 | 0.00038 |
| (SEQ ID NO. 14) | | | | |

### Role of Tau Acetylation and Use as a Biomarker

The results presented herein demonstrate tau acetylation as a post-translational modification that may regulate normal tau function and suggests a role in pathological tau aggregation in AD and related tauopathies. Increased tau acetylation on K280 could impair tau interactions with MTs and provide increased pools of cytosolic tau available for pathological PHF aggregation. Consistent with this, K280, located in the inter-repeat region, was identified previously as one of three lysine residues most critical in modulating tau-MT interactions (Goode & Feinstein, 1994, J. Cell Biol. 124:769-782). Moreover, K280 acetylation was not observed in tau fibrils assembled from recombinant T40 because this residue is buried within the fibril core. Thus, the present data support the notion that K280 acetylation occurs before tau fibrillization into PHFs and therefore is likely a pathological event. Indeed, K280 acetylation was detected in all 4R- and 3R/4R-tauopathies that were examined herein, but was absent in 3R predominant PiD brains or soluble tau fractions of normal control samples.

Acetylation of tau aggregates was associated with hyperphosphorylated, ThS-positive tau inclusions in both Tg mouse models and human tauopathies. This implies that negative regulation of tau function could occur via phosphorylation and acetylation events alone or in combination. Tau is extensively phosphorylated on at least 25 distinct serines/threonines residues, most of which are outside the MT-binding repeat region. In contrast, tau acetylation in cells was specifically detected on four lysines, three of which are located within the MT-binding repeats. Thus, tau acetylation and phosphorylation may represent specific modifications required to dynamically regulate tau function. While not wishing to be bound by any particular theory, one possibility is that increased tau phosphorylation could facilitate tau acetylation within the MT repeat domain to further impair tau binding to MTs, suggesting complex regulation of tau-MT dynamics. A more comprehensive analysis of acetylated and phosphorylated sites in diseased brains could provide insight into the potential cooperative regulation of tau post-translational modifications during disease pathogenesis.

K280 resides in the double lysine motif, 275-VQIINKK-281 (SEQ ID NO. 3), which is critical for MT binding. Indeed, this motif is conserved in other tau family members including MAP2 and MAP4, indicating reversible acetylation could represent a conserved mechanism to regulate the MT-binding activity of other MAPs in addition to tau. Although protein acetylation has been extensively studied in the context of histones and gene transcription, proteomics approaches have identified acetylated proteins in the cytoplasm and other organelles (Choudhary, et al., 2009, Science 325:834-840). Several acetyl-transferases and deacetylases (HDACs) are localized to the cytoplasm and MTs (Creppe, et al., 2009, Cell 136:551-564; Hubbert, et al., 2002, Nature 417:455-458), suggesting acetylation may represent a general mechanism to regulate cytoskeletal dynamics and coordinate cytoplasmic signaling events.

The present IHC and biochemical studies suggest that K280 acetylation may be more disease specific than phosphorylated tau in tauopathies. Thus, monitoring acetylated tau levels may represent a potential biomarker for the diagnosis of AD and related neurodegenerative tauopathies. Moreover, the identification of tau acetylation in tauopathies provides a framework for both biomarker development and for therapeutic targeting of tauopathies via the acetylation/deacetylation machinery.

### Example 2

### The Microtubule-Associated Tau Protein has Intrinsic Acetyltransferase Activity

The data presented in Example 1 establish that tau is extensively post-translationally modified by lysine acetylation, which impairs normal tau function and promotes pathological aggregation. Identifying the enzymes that mediate tau acetylation could provide targets for future therapies aimed at reducing acetylated tau burden. The results of the experiments described in the present example establish that mammalian tau proteins possess intrinsic enzymatic activity capable of catalyzing self-acetylation. Functional mapping of tau acetyltransferase activity followed by biochemical analysis reveals that tau utilizes catalytic cysteine residues in the microtubule binding domain to facilitate tau lysine acetylation, suggesting a mechanism similar to that employed by MYST-family acetyltransferases. The identification of tau as an acetyltransferase provides a framework to further understand tau pathogenesis and highlights tau enzymatic activity as a potential therapeutic target.

The experiments presented herein were designed to uncover the dominant mechanism(s) that mediates tau acetylation, which opens up new therapeutic avenues to reduce tau aggregation and potentially ameliorate disease pathogenesis. In these experiments, tau itself is identified as a bona fide acetyltransferase with sequence and functional similarities to the MYST family of acetyltransferases. Biochemical and kinetic studies indicate that tau catalyzes robust self-acetylation (auto-acetylation) mediated by a pair of catalytic cysteine residues residing within the MT-binding domain. These studies suggest that prolonged activation of tau acetyltransferase activity represents a novel pathway that mediates tau pathogenesis, and highlights tau enzymatic function as a potential therapeutic target for Alzheimer's disease and related tauopathies characterized by robust accumulation of acetylated tau pathology.

The materials and methods use in the experiments disclosed herein are now described.

Recombinant Tau In Vitro Methods

Acetyltransferase assays using purified tau proteins were performed as previously described and subjected to autoradiography or immunoblotting using the following anti-tau antibodies: anti-tau polyclonal (Dako, A0024, 1:10,000), anti-acetyl lysine (Cell Signaling, #9441, 1:1000), anti-tau monoclonal (T46, 1:1000), anti-mouse tau (T49, 1:1000), anti-acetylated tau (Ac-K280, 1:1000), anti-acetylated tau (Ac-K369, 1:1000). For tau acetyltransferase inhibition studies, tau proteins were pre-incubated with 0.5 mM N-ethyl-maleimide (NEM), 4 mM iodoacetamide (IA), or increasing concentrations of either tubulin (5-10 μM) or pre-formed microtubules (5 μM). Mass spectrometry (nanoLC nanospray MS-MS) analysis was performed at the Penn proteomics core facility, which identified tau auto-acetylated lysine residues. In-gel acetyltransferase assays were performed using a variation of the in-gel HAT protocol previously described by Brownell et al. (1995, Proc Natl Acad Sci USA 92:6364-8). Briefly, tau proteins (5-50 μg total protein) were electrophoresed on a 15% SDS gel followed by extensive washing in Buffer 1 (50 mM Tris, pH 8.0, 20% isopropanol, 0.1 mM EDTA, 1 mM DTT). Gels were equilibrated in Buffer 4 (50 mM Tris pH 8.0, 10% glycerol, 0.1 mM EDTA, 1 mM DTT) followed by incubation for 2 hr with Buffer 4 containing 5 μCi [³H]-acetyl-CoA (1-10 Ci mmol⁻¹, Perkin Elmer). Gels were then washed in 5% trichloroacetic acid several times to reduce background, dried down, and exposed to film for 2 weeks. For tau trans-acetylation western blotting, 2N4R-tau and 2N4R-tau-2CA mutant (0.3 μM) were incubated with increasing concentrations of enzymatically active tau-K18 or deficient tau-K18-2CA (2-7 μM) for subsequent analysis of inter-molecular tau acetylation. Reaction products were readily distinguished between 2N4R-tau (Mr ˜65 kD) and tau-K18 (Mr ˜15 kD) by immunoblotting using acetylated tau (Ac-K280) and total tau (E10) antibodies. Total tau immunoreactivity using polyclonal anti-tau E10 was reduced upon tau acetylation.

Cell and Tissue-Based Acetylation Assays

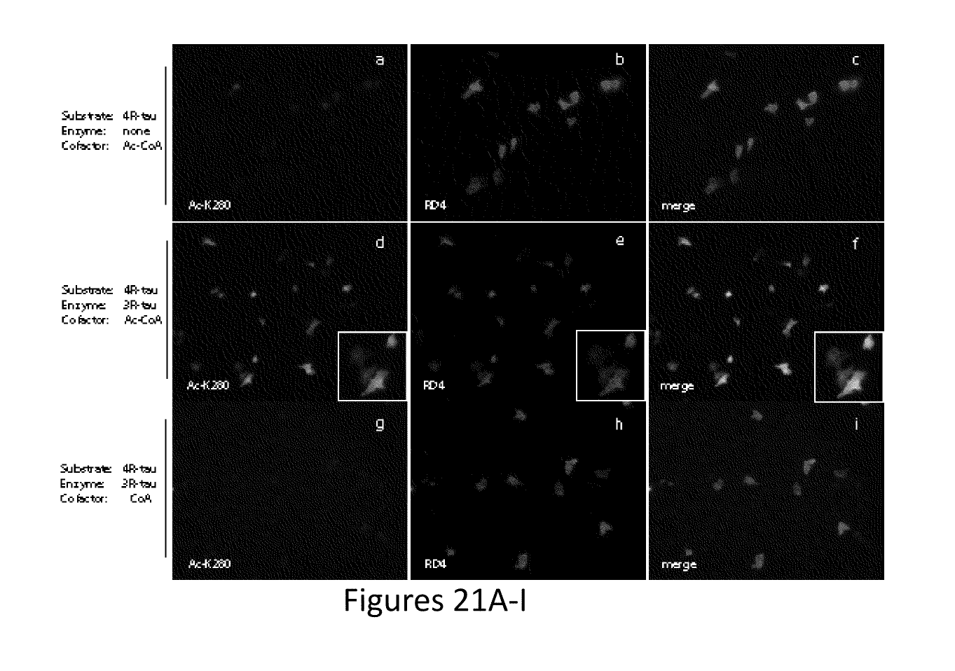

Cell-based tau-T40 (2N4R) acetyltransferase assays were performed using immunoprecipitated tau proteins (tau mAb T46+T14) bound to protein A and G beads followed by addition of [¹⁴C]-Acetyl-CoA for 2 hr. Acetylated tau proteins bound to beads were analyzed by SDS-PAGE and Coomassie staining following by phosphorimaging using Storm software. For acetyltransferase assays using brain lysates, boiled mouse lysates or salt-extracted human lysates were dialyzed into PBS and supplemented with Tris pH 8.0 to a final concentration of 50 mM. CoA or Acetyl-CoA was added to 1 mM and incubated for 2-hr at 37° C. followed by immunoblotting using the indicated anti-tau antibodies. Cell-based K18 acetylation was performed using K18-P301L (K18-PL), mutants lacking cysteines (K18-PL-C291-322A), or mutants containing phospho-mimic mutations (K18-PL-S262-356E). Transfected cells were treated overnight with 10 μM 3-methyladenine (3MA) and 50 μM sodium arsenite, where indicated, followed by sequential biochemical extraction of soluble (RIPA-solubilized) and insoluble (UREA-solubilized) fractions (Cohen et al., 2012, EMBO J 31, 1241-52). Immunoblotting of cell lysates was performed using the following primary antibodies in addition to those indicated above for recombinant methods: total tubulin (Sigma, DM1A, 1:2000), anti-acetylated tubulin (Sigma, T6793, 1:1000), acetylated histone H3K18 (Active Motif, #39755, 1:1000), acetylated histone H3K9 (Millipore, 07-352, 1:1000), phospho-tau 12E8 (Elan Pharmaceuticals, 1:1000), GAPDH (Advanced Immunochemical, 2-RGM2, 1:3000), M2-FLAG (Sigma, F1804, 1:2000). In-trans immunofluorescence (trans-IF) was performed using primary hippocampal neurons isolated from CD1 embryos (Charles River, Wilmington, Mass.) following the NIH Guide for the Care and Use of Experimental Animals and were approved by the University of Pennsylvania Institutional Animal Care and Use Committee. Neurons were grown on cover slips for 21 days in culture followed by fixation in 4% paraformaldehyde and permeabilization in 0.2% Triton for 15 min. Cover slips were incubated with 0.5 mg ml⁻¹ tau-K19 or tau-K19-C322A mutant proteins in the presence of 1 mM CoA or Acetyl-CoA for 2 hr followed by extensive washing in PBS and immunostaining analysis using Ac-K280 and total mouse anti-tau (T49) antibodies. A similar analysis was performed by incubating purified ON3R-tau with 2N4R-tau transfected QBI-293 cells (FIG. 21).

Antibody Generation

Polyclonal anti-acetyl tau Lys369 antibodies were generated similar to Lys280 as described herein using the tau peptide C-GGNKKIE [SEQ ID NO: 15] (United Peptide Corp., Bethesda, Md.) containing acetylated K369 to immunize rabbits (Pocono Rabbit Farm and Laboratory Inc., Canadensis, Pa., USA). Double affinity purification was performed using native and acetylated peptides sequentially using Sulfolink columns (Pierce Biotechnology). Site specificity of Ac-K369 was confirmed in vitro and in cells using tau proteins lacking residue K369.

Tau Auto-Acetylation Filter Binding Assays

Kinetic Analysis.

Reactions were assembled in 50 μl of reaction buffer containing 50 mM Tris pH 8.0, 100 μM EDTA, and 10% glycerol. Unless specifically noted, reactions contained 25 μM of either WT tau-K18 or tau-K18-2CA mutant. The reactions were initiated by the addition of [³H]-acetyl-CoA (2.35 Ci mmol⁻¹, Perkin Elmer) at concentrations ranging from 2.5 μM to 1 mM, incubated at 37° C., and allowed to proceed for 1 hr (unless otherwise noted). Reactions were stopped by the addition of 10 μl of 3 mM CoA, and 25 μl of the reaction was then spotted to a P81 filter paper (Whatman), washed in 10 mM HEPES pH 7.5 three times, and dried using acetone. Incorporated [³H]-acetyl-CoA was then measured using the Perkin Elmer Tri-Carb 2800TR Liquid Scintillation Analyzer, and the molar amount of acetyl groups incorporated into tau were calculated from a standard curve of the radiolabeled acetyl-CoA.

Inhibition Analysis.

Reactions were performed in the buffer described above and at 25 μM tau. Reactions included between 1 and 4000 μM of the inhibitors: CoA, acetonyl-CoA, or pantothenic acid. All reactions were initiated by the addition of 450 μM [³H]-acetyl-CoA (2.35 Ci mmol⁻¹, Perkin Elmer), incubated for 1 hr at 37° C., and stopped by spotting 25 μl of the reaction to the P81 filter paper. Radiolabeled incorporation was measured as stated above. All data were analyzed using GraphPad Prism to determine kinetic parameters and IC₅₀ values.

The results of the experiments presented herein are now described.

Tau Proteins Possess Auto-Acetyltransferase Activity

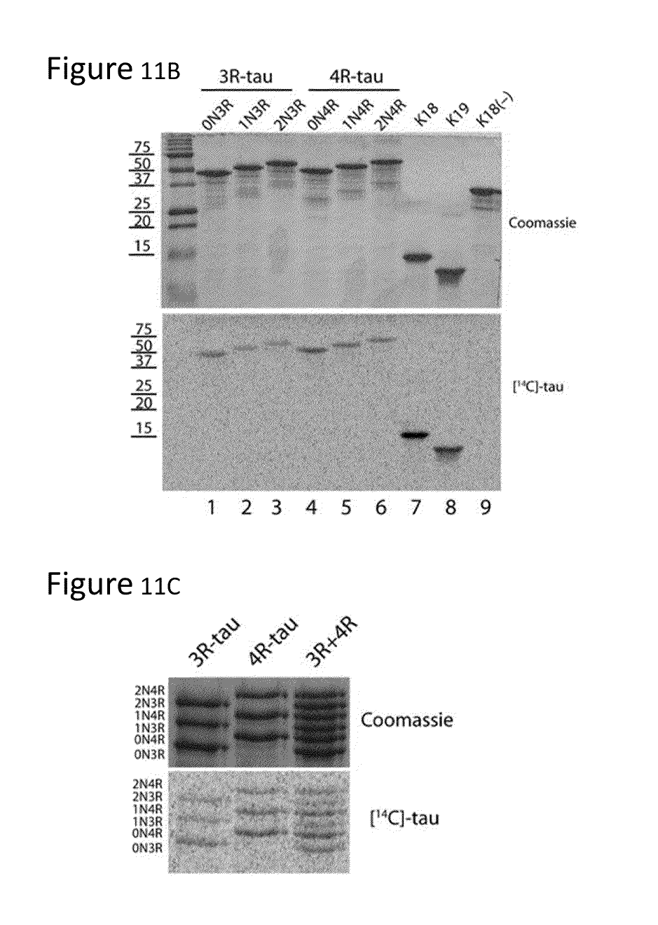

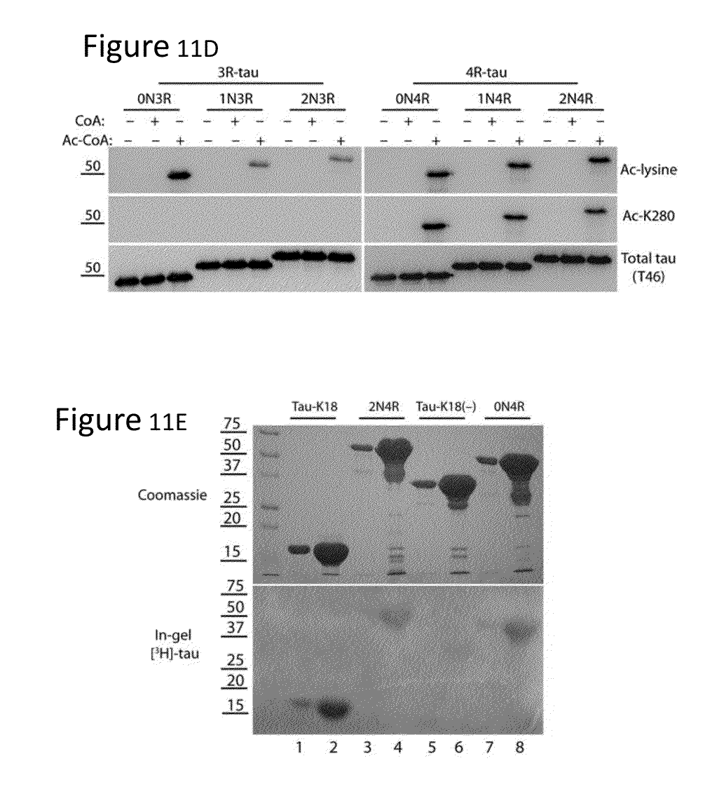

To characterize a putative tau acetyltransferase activity, full-length tau proteins containing either 3 or 4 MT-binding repeats (i.e. 3R-tau and 4R-tau), tau fragments containing only 3 or 4 repeats (i.e. tau-K19 and tau-K18), or tau protein lacking the repeats (tau-K18(−)) were purified. Acetyltransferase activity was then assessed in the presence of [¹⁴C]-acetyl-CoA (see FIG. 11a for a schematic of all tau proteins used in this study). As shown in FIG. 11b, all 3R-tau and 4R-tau proteins possessed robust auto-acetylation activity, which was prominently observed with repeat-containing tau-K19 and tau-K18 fragments (FIG. 11b). Notably, the tau-K18(−) protein lacking repeat regions had no detectable activity (FIG. 11b), suggesting that enzymatic activity resides within the MT-binding regions. Acetyltransferase activity was similarly detected upon co-incubation of 3R-tau or 4R-tau isoforms separately, or all 6 tau isoforms co-incubated together (FIG. 11c).

To confirm auto-acetylation of lysine residues, tau proteins were incubated with CoA or acetyl-CoA followed by immunoblotting using pan anti-acetyl-lysine and anti-acetyl-tau-K280 (Ac-K280) antibodies (See Example 1; Irwin et al., 2012, Brain 135:807-18). Consistent with the autoradiography results, all 6 tau isoforms as well as the tau-K18 fragment showed robust auto-acetylation as revealed by anti-acetyl-lysine immunoblotting (FIG. 11d). As expected, the Ac-K280 antibody detected only auto-acetylated 4R-tau isoforms since residue K280 within the 2^{nd} repeat is present in 4R-tau but not 3R-tau. To identify the full extent of auto-acetylated lysine residues within the repeat region, mass spectrometry was conducted on auto-acetylated tau-K18 and a subset of acetylated lysines was identified that overlap with previously identified CBP-mediated acetylated lysines (Example 1; Min et al., 2010, Neuron 67:953-66). Thus, both 3R- and 4R-tau proteins showed robust acetyltransferase activity.

To rule out the possibility that a contaminating acetyltransferase accounts for the observed acetyltransferase activity in these highly purified recombinant tau proteins, an in-gel assay that was previously used to assess histone acetyltransferase (HAT) activity was conducted (Brownell et al., 1995, Proc Natl Acad Sci USA 92:6364-8; Brownell et al., 1999, Methods Mol Biol 119:343-53). As shown in FIG. 11e, a gel containing two different concentrations of the active tau-K18 fragment, ON4R- or 2N4R-tau proteins, or inactive tau-K18(−) was incubated with radio-labeled [³H]-acetyl-CoA followed by autoradiography. In-gel tau acetyltransferase activity co-migrated prominently with tau-K18 and to a lesser extent with both full-length tau proteins, while tau-K18(−) showed minimal detectable acetyltransferase activity (FIG. 11e). Therefore, the in-gel assay supports the interpretation that tau auto-acetyltransferase activity is mediated by the MT-binding repeat regions.

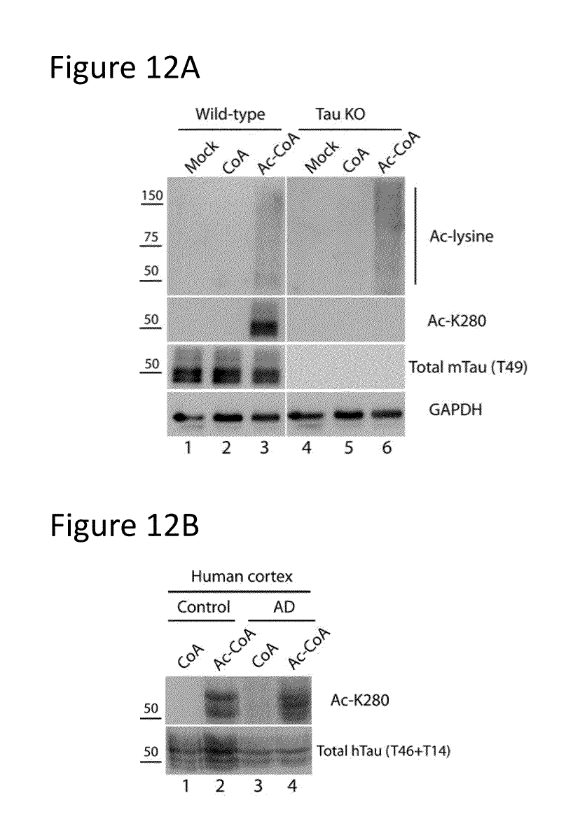

To evaluate if brain-derived tau can undergo de novo auto-acetylation, extracts from cortical brain homogenates of wild-type (WT) and tau knock-out (KO) mice were boiled, which inactivated any potentially contaminating acetyltransferases and simultaneously enriched for heat-stable tau proteins. WT or tau KO brain extracts were incubated with CoA or acetyl-CoA and tau acetylation was evaluated by immunoblotting using acetylation specific antibodies (FIG. 12a). Strikingly, incubation of WT extracts with acetyl-CoA, but not CoA, led to a dramatic accumulation of acetylated mouse tau as detected with anti-Ac-K280 and a modest increase in total acetylated proteins (FIG. 12a). As expected, acetyl-CoA dependent tau acetylation was not detected in tau-KO brain extracts although a modest increase in total acetylated proteins was also observed (FIG. 12a). Soluble tau proteins derived from high-speed supernatants from frontal cortex homogenates of control and Alzheimer disease brain were evaluated, which were similarly capable of de novo auto-acetylation as detected by anti-Ac-K280 without detectable disease-specific differences (FIG. 12b).

Tau Acetyltransferase Activity is Mediated by Cysteines

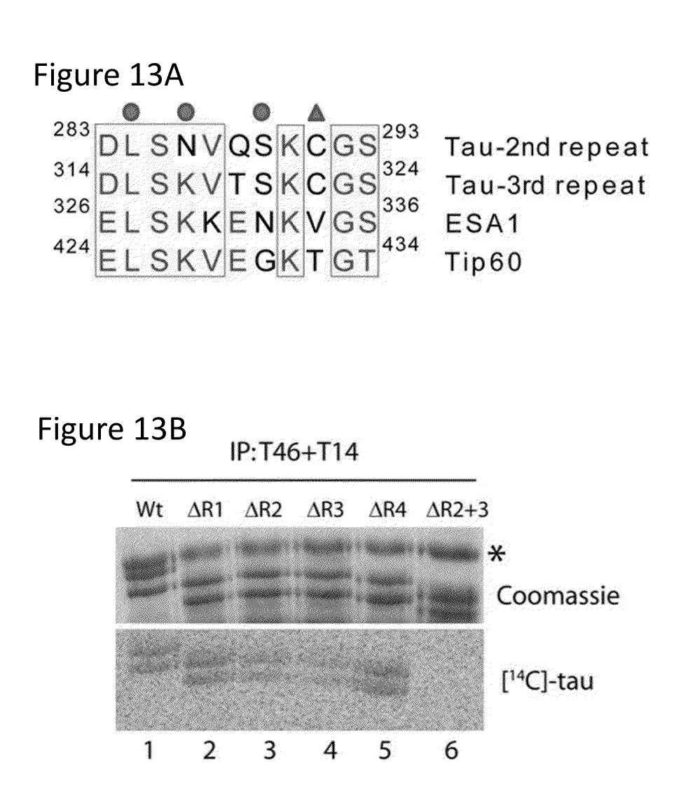

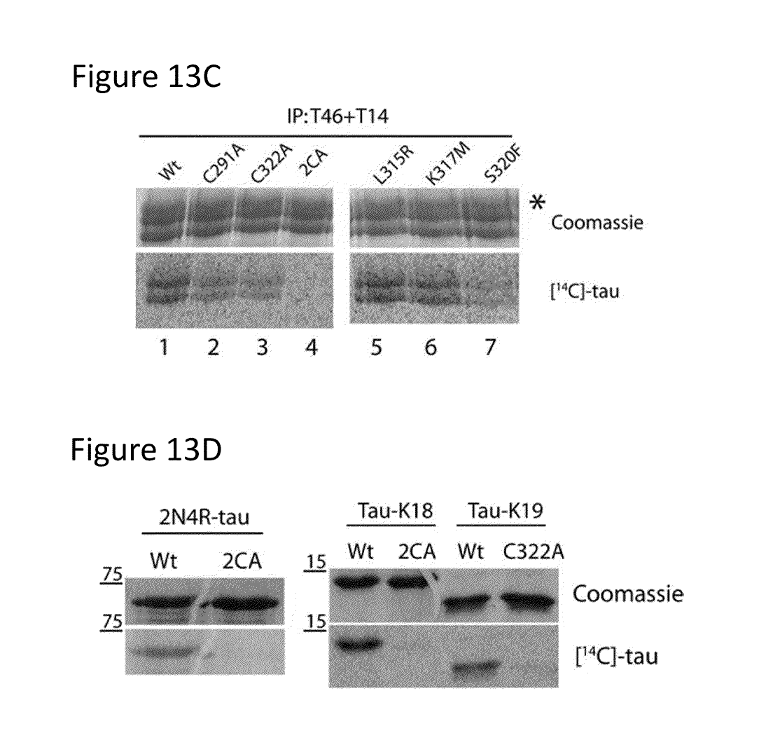

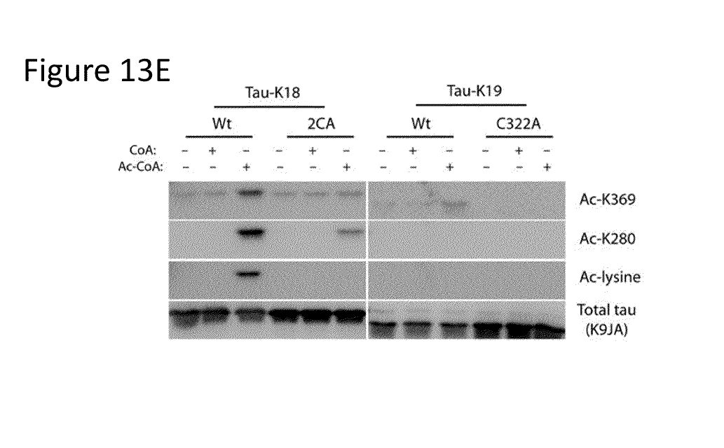

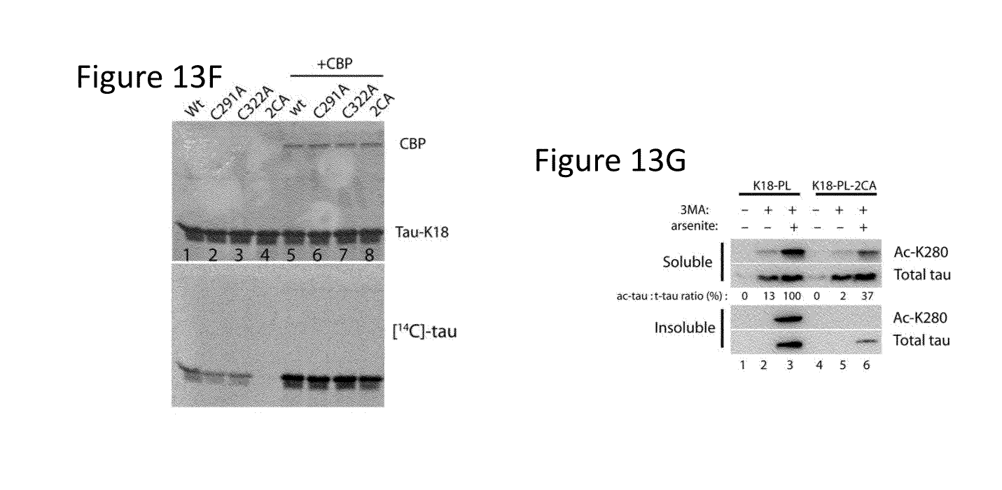

Sequence similarities between tau and known acetyltransferases were assessed. Several variants of the well characterized motif-A were identified within the repeat region (G-X-G), which could participate in direct acetyl-CoA binding and catalysis (Kawahara et al., 2008, Mol Microbiol 69:1054-68; Sterner and Berger, 2000, Microbiol Mol Biol Rev 64:435-59; Sternglanz and Schindelin, 1999, Proc Natl Acad Sci USA 96:8807-8). Additionally, two regions were identified in the 2^{nd} and 3^{rd} repeats with ˜75% sequence homology to the MYST-family acetyltransferases ESA1 and Tip60 (refs. 12,13, see boxed regions in FIG. 13a). The minimal MYST region shown represents part of a larger conserved core acetyltransferase domain, suggesting that a similar region in tau could facilitate auto-acetylation. To investigate this possibility, tau acetyltransferase activity was mapped using a cell-based in vitro acetylation assay in which HEK293 cells were transfected with a series of tau expression plasmids lacking individual repeats (ΔR1-R4) or lacking both repeats 2 and 3 in combination (ΔR2+3) (FIG. 13b). Over-expressed tau proteins were immunoprecipitated from cell lysates and incubated with [¹⁴C]-acetyl-CoA. Deletion of R2 or R3, but not R1 or R4, led to a modest reduction in tau acetyltransferase activity (FIG. 13b). Consistent with two catalytic regions, deleting both R2 and R3 in combination completely abrogated tau acetyltransferase activity (FIG. 13b), thereby mapping tau enzymatic activity to repeats R2 and R3 that contain sequence homology to acetyl-CoA binding regions of MYST acetyltransferases.

Previous structural and functional studies demonstrated that MYST-family acetyltransferases can utilize an active site acetyl-cysteine intermediate to facilitate acetyl group transfer to terminal lysines (Yan et al., 2002, Nat Struct Biol 9:862-9; Decker et al., 2008, Genetics 178:1209-20; Yang et al., 2012, PLoS One 7:e32886). Sequence analysis within tau repeats R2 and R3 revealed an R2-cysteine (Cys291) and a comparable R3-cysteine (Cys322) that are notably absent in repeats R1 and R4 (FIG. 13a, see blue triangle), suggesting tau cysteines may represent catalytic residues that mediate acetyltransferase activity. To evaluate this possibility, Cys291 and Cys322 were mutated to alanines individually or in combination and tau proteins were analyzed by immunoprecipitation followed by acetyltransferase assays. Individual cysteine mutants retained tau enzymatic activity but mutation of both cysteine residues abolished auto-acetylation activity (FIG. 13c). Analysis of several pathogenic FTDP-17 tau mutations within this region demonstrated that the S320F mutation, within close proximity to Cys322, showed a modest reduction in auto-acetylation activity (FIG. 13c). A panel of full-length and repeat-containing tau proteins lacking cysteines (2N4R-tau-2CA, tau-K18-2CA, or tau-K19-1CA) showed abrogated acetyltransferase activity by both autoradiography (FIG. 13d) and immunoblotting using acetylated tau-specific antibodies (FIG. 13e). Although cysteine-deficient tau had impaired auto-activity, the tau-K18-2CA mutant was readily acetylated by active recombinant CBP (FIG. 13f), suggesting that direct lysine acetylation mediated by other acetyltransferases is not generally impaired in the absence of tau cysteines.

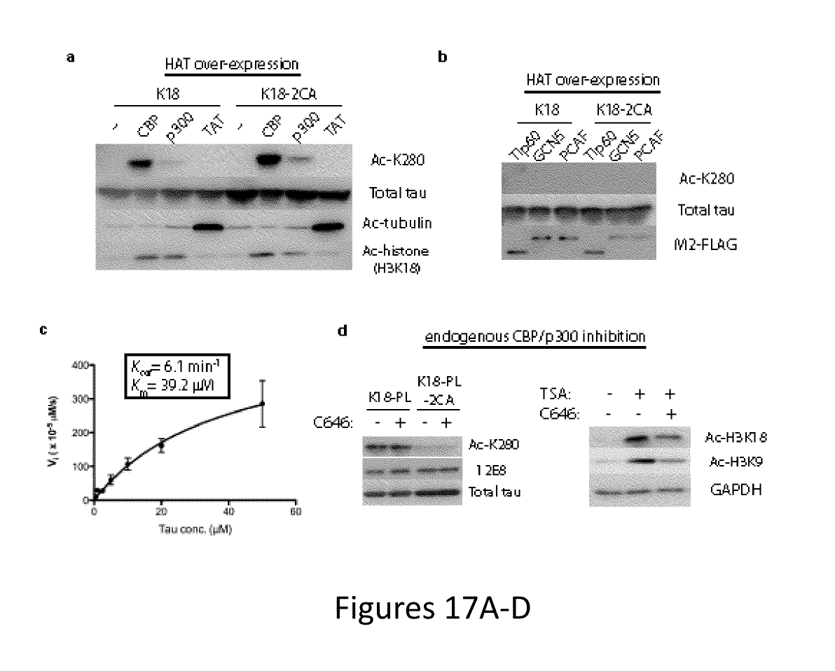

To evaluate tau acetyltransferase activity in cells, a cell-based model of tau acetylation in the absence of ectopically expressed CBP was developed. Given that Lys280-acetylated tau is only detectable under pathological conditions in diseased brain (Example 1), a genetic and pharmacological approach to increase the cellular pool of acetylated tau was devised. Cells expressing a tau-K18 fragment harboring the pathogenic P301L mutation (K18-PL) were treated with the autophagy inhibitor 3-methyladenine (3MA), which impairs K18-PL degradation (Wang et al., 2009, Hum Mol Genet 18:4153-70), as well as the oxidative stressor sodium arsenite, previously shown to increase tau phosphorylation (Giasson et al., 2002, Biochemistry 41:15376-87). In combination, 3MA-arsenite treatment resulted in prominent pathological tau hallmarks, including the accumulation of acetylated and biochemically insoluble K18-PL protein (FIG. 13g). Importantly, the increased K18-PL acetylation and shift in solubility was dramatically abrogated by an enzyme-deficient K18-PL lacking Cys291 and Cys322 (FIG. 13g). Thus, tau cysteines are required for auto-acetylation and insoluble tau accumulation in a cell-based model of tau pathogenesis. Supporting a minimal role for CBP in K18-PL acetylation, pharmacological inhibition of endogenous CBP with the compound C646 had no effect on K18-PL acetylation levels (FIG. 17d).

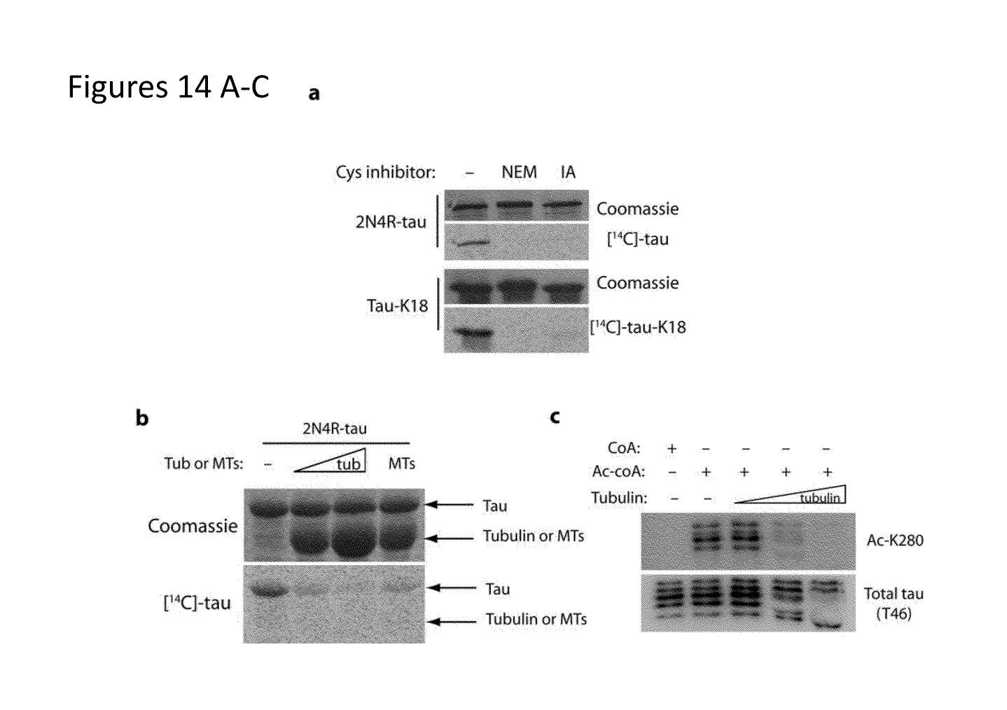

A pharmacological approach to confirm the importance of Cys291 and Cys322 in mediating tau acetyltransferase activity was used. Pretreatment of 2N4R-tau and tau-K18 with either N-ethyl-maleimide (NEM) or iodoacetamide (IA), compounds that bind free cysteine thiol groups, resulted in the abrogation of 2N4R-tau or tau-K18 auto-acetylation activity (FIG. 14a). Since cysteine-containing R2 and R3 repeats bind tubulin, the question of whether the presence of tubulin affected tau enzymatic activity was assessed. Indeed, increasing concentrations of either monomeric tubulin or pre-formed microtubules dramatically reduced tau acetyltransferase activity (FIGS. 14b and 14c), suggesting that only free unbound tau proteins, but not those associated with tubulin, are capable of auto-acetylation. These data suggest that robust tau acetyltransferase activity occurs under conditions that favor reduced tau binding to tubulin, thus exposing free cysteine residues for catalysis.

Kinetic Analysis of Tau Acetyltransferase Activity

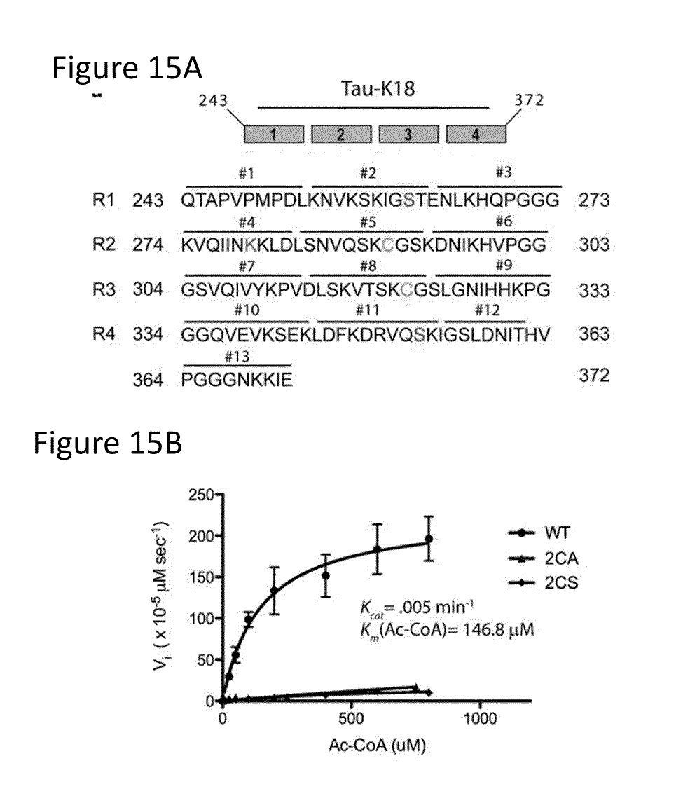

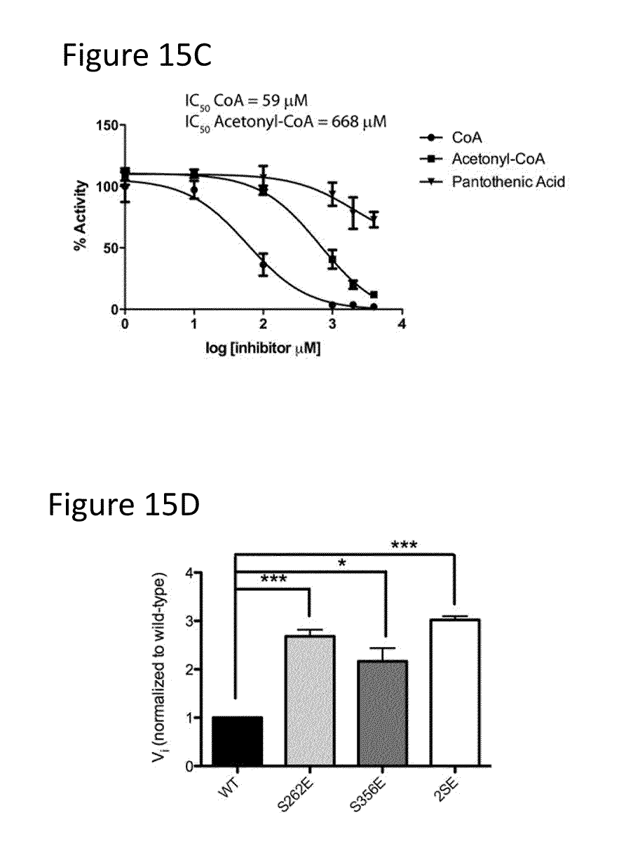

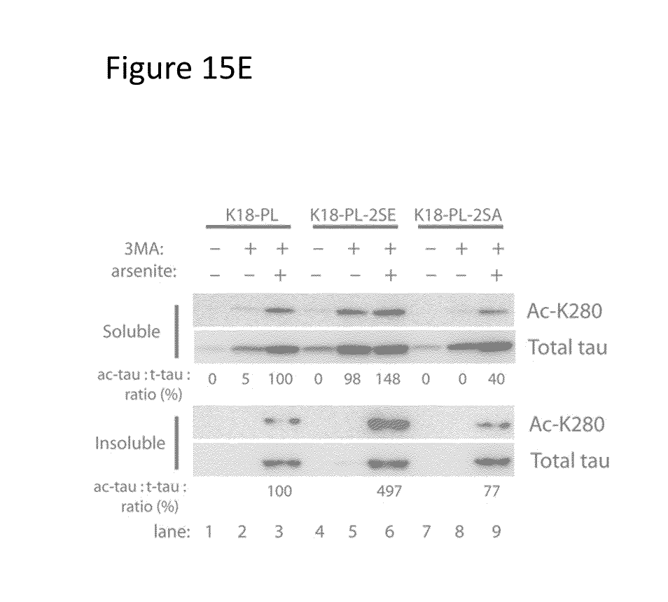

To quantify tau acetyltransferase activity, filter binding assays were conducted using recombinant wild-type tau-K18 or cysteine mutants (tau-K18-2CA and tau-K18-2CS) containing either alanine or serine substitutions (see FIG. 15a for tau-K18 sequence). A saturation plot of reaction velocity against acetyl-CoA concentration revealed that tau-K18 acetyltransferase activity follows classic Michaelis-Menten behavior, while 2CA and 2CS mutant activity was impaired, as expected (FIG. 15b). Although the calculated turnover rate for tau-K18 (K_{cat}=0.005 min⁻¹) is quite low in comparison to known nuclear acetyltransferases, it is comparable to the recently reported cytoplasmic α-tubulin acetyltransferase (αTAT-1, K_{cat}=0.037 min⁻¹) (Shida et al., 2010, Proc Natl Acad Sci USA 107:21517-22), indicating distinct kinetic differences between nuclear and cytoplasmic acetyltransferases (see Table 4 for kinetic comparisons among nuclear and cytoplasmic acetyltransferases). Consistent with acetyl-CoA binding to tau, inhibition of tau acetyltransferase activity was readily observed by cofactor competition assays, in which titration of increasing amounts of CoA or the structurally related acetyl-CoA analog, acetonyl-CoA, inhibited acetyl-CoA-dependent tau-K18 acetyltransferase activity (FIG. 15c), with IC₅₀ values of 59 μM and 668 μM for CoA and acetonyl-CoA, respectively. As a negative control, an acetyl-CoA precursor, pantothenic acid, did not show significant inhibition of tau activity.

TABLE 4
Comparison of enzyme kinetics among acetyltransferases.

| | Substrate | Kcat | Km [protein] | Km [AcCoA] | Reference |
|---|---|---|---|---|---|
| p300 | H4-21 | 27.6/min | 1.210.54 uM | 1.2410.19 uM | J Biol Chem. 2001 Sep 7; 276\|36\|: 33721-9 |
| ESA1 | H3p19 | 7.32/min | 0.92210.1543 uM | 9.113611.48 uM | Nal. Struct Biol. 2002 Nov; 9\|11\|: 862-9 |
| Rtt109 + Vps75 | H3 | 22.5/min | 8.510.7 uM | 8.012.0 uM | Nal. Struct Mal Biol. 2008 Sep; 15\|9\|: 998 |
| Tau | ND | ND | ND | ND | |
| TAT | microtubules | 0.037/min | 1.6 uM 10.36 | 2.210.2 uM | Proc Natl Acad Sci USA. 2010 Dec |
| | | | | | 14; 107\|50\|: 21517-22 |

| | Substrate | Kcat [auto] | Km AcCoA [auto] | Reference |
|---|---|---|---|---|
| p300 | auto | ND | 3310.8 uM\|AcCoA\| | J Biol Chem. 2000 Jul 21; 275\|29\|: 21953-9 |
| ESA1 | auto | ND | 9.4 uM \|AcCoA\| | EMBO J. 2011 Oct 21; 31\|1\|: 58-70 |
| Rtt109 + Vps75 | auto | .12/min | 912 uM | J Biol Chem. 2011 Jul 15; 296\|28\|: 24694-701 |
| Tau | auto | .005/min | 146.8 uM | this study |
| TAT | ND | ND | ND | |

Kinetics parameters for nuclear and cytoplasmic acetyltransferases are listed. Kinetic values for known substrates including histones and microtubules are shown in the top panel, while auto-acetylation kinetics are shown in the bottom panel. Auto indicates auto-acetylated substrate, and ND indicates the value was not determined.

In addition to catalytic cysteines, the tau domains and regulatory elements that are critical for substrate binding or catalysis were analyzed. A panel of tau-K18 mutant proteins containing sequential 10-amino acid deletions was generated and kinetic analysis to determine Kₘ values relative to wild-type tau-K18 was conducted. While many of the tau deletion proteins analyzed retained enzymatic function comparable to wild-type (Kₘ=147 μM), mutants #3 and #10, located within repeats 1 and 4, respectively, had ˜20-40 fold elevated Kₘ values (Table 5, see asterisks), which indicated that distinct structural regions spanning tau-K18 are likely required for substrate binding or catalysis.

TABLE 5
Kinetic analysis of tau-K18 proteins containing sequential deletions

| | Kₘ (μM) |
|---|---|
| 1 | 332 |
| 2 | 370 |
| *3 | 2758 |
| 4 | 1215 |
| 5 | N.D. |
| 6 | 1150 |
| 7 | 716 |
| 8 | 141 |
| 9 | 319 |
| *10 | 5404 |
| 11 | 421 |
| 12 | 250 |
| 13 | 273 |
| WT | 147 |

WT, wild-type,
N.D., not determined

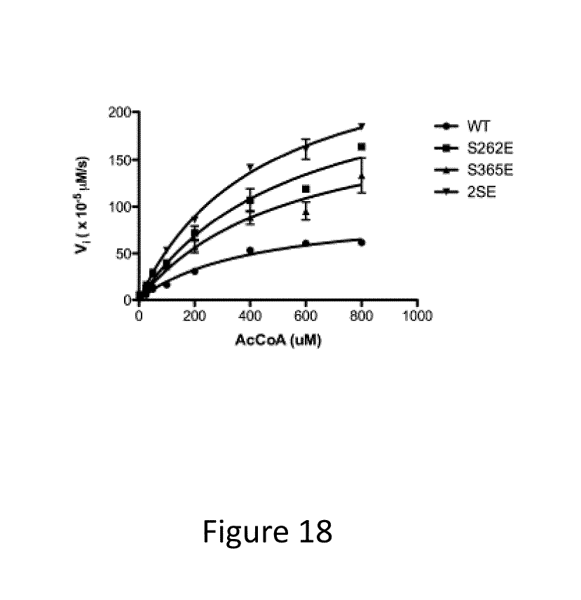

Given that pathological tau aggregates in diseased brain are both hyper-phosphorylated and acetylated, the question of whether tau phosphorylation regulates tau acetyltransferase activity was asked. Tau is phosphorylated on several KXGS motifs within the repeats, most notably Ser262 and Ser356 comprising the 12E8 epitope (Drewes et al., 1997, Cell 89:297-308), thus providing regulatory cross-talk that could activate tau acetyltransferase activity. To examine this possibility in vitro, phospho-mimic tau-K18 proteins containing single S262E or S356E serine-to-glutamic acid mutations or a double S262E S356E mutation (2SE) were generated and acetyltransferase assays were conducted. Unexpectedly, increased acetyltransferase activity was observed with all phospho-mimic proteins, including the 2SE mutant that showed a ˜3-fold activation (FIG. 15d and FIG. 18). These results were confirmed in a cell-based model, in which expression of a phospho-mimic K18-PL mutant (K18-PL-2SE), but not a control mutant containing serine-to-alanine substitutions (K18-PL-2SA), resulted in elevated acetylated tau levels in response to either 3MA alone or 3MA-arsenite in combination (FIG. 15e). Thus, pseudo-phosphorylation within the MT-binding domain is sufficient to increase tau-K18 acetyltransferase activity.

Mechanism of Tau-Mediated Acetyltransferase Activity

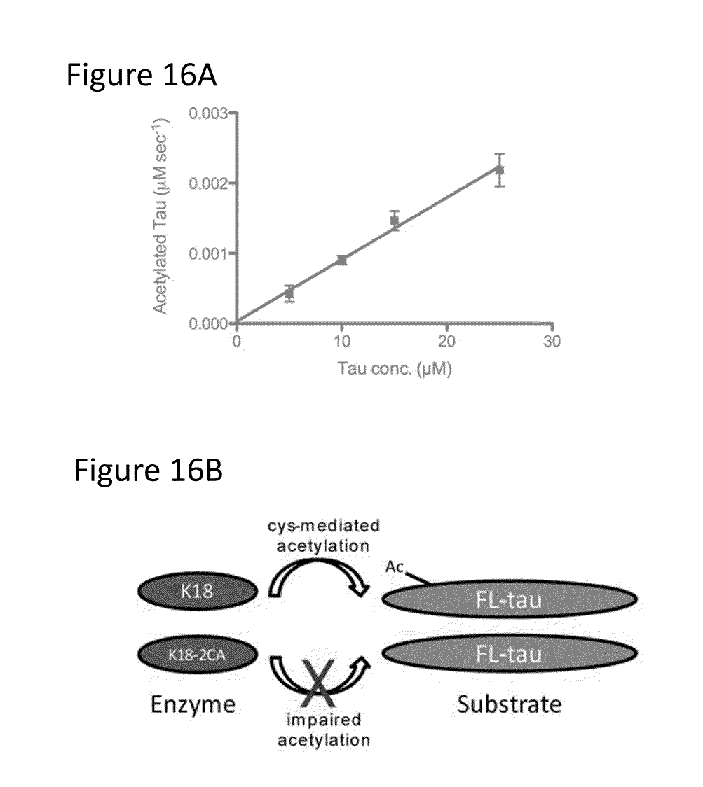

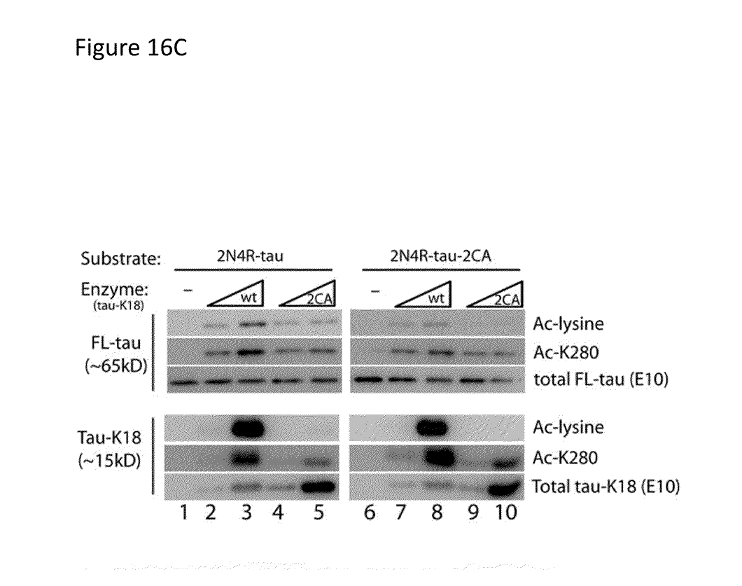

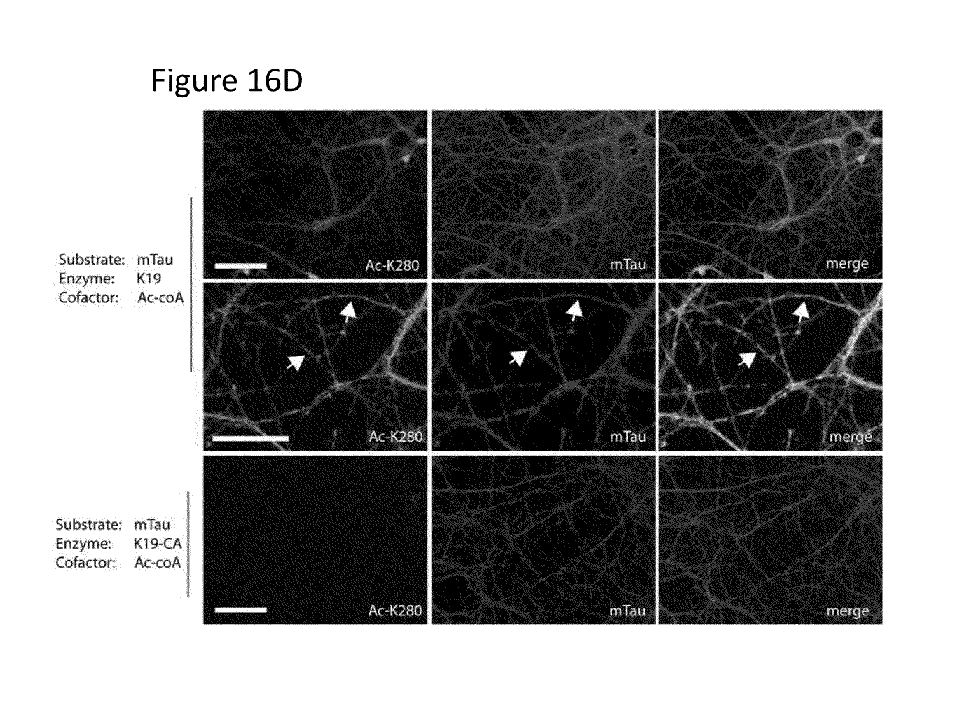

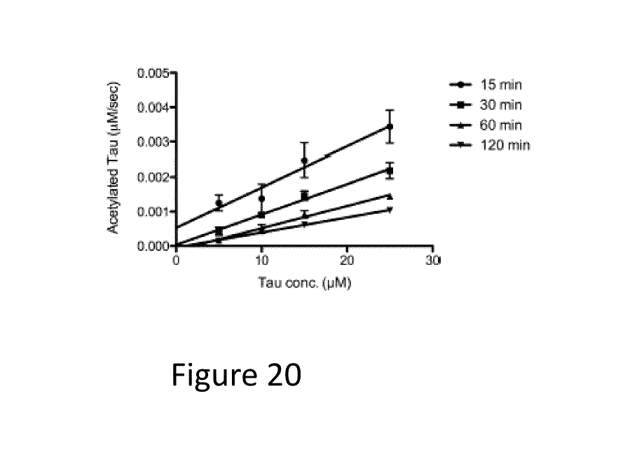

To examine putative tau substrates, whether tau directly acetylated microtubules, similar to that described for the tubulin acetyltransferase αTAT-1 (Shida et al., 2010, Proc Natl Acad Sci USA 107:21517-22; Akella et al., 2010, Nature 467:218-22). However, using a PTK2 cell culture model, tau was unable to mediate microtubule acetylation in contrast with α-TAT-1 (FIG. 16). Furthermore, tau was unable to restore microtubule acetylation in Hela cells lacking αTAT-1, further supporting a minimal role, if any, for direct tau-mediated acetylation of microtubules. The focus was therefore on tau itself as a major auto-acetylated substrate. Experiments were conducted to determine whether tau auto-acetylation occurred via an intra- or inter-molecular reaction mechanism. To distinguish between these possibilities, filter binding assays were conducted to determine the initial rate of tau acetylation as a function of tau concentration. A linear correlation of the reaction rate (μM sec⁻¹) vs. tau concentration (μM) was observed at all timepoints examined (FIG. 16a and FIG. 20), supporting a predominantly first-order intra-molecular reaction mechanism. However, these results did not exclude potential inter-molecular interactions as a contributing factor to tau auto-acetylation. Therefore, an enzyme-substrate acetylation assay was used in which increasing concentrations of enzymatically active tau-K18 or enzyme-deficient tau-K18-2CA mutant were incubated with full-length 2N4R-tau substrate (FIG. 16b). Auto-acetylation of 2N4R-tau (Mr˜65 kD) was not detected with anti-acetyl-lysine and Ac-K280 antibodies at the low substrate concentrations used (FIG. 16c). However, increasing amounts of tau-K18 (Mr˜15 kD) increased the acetylation of both 2N4R-tau and 2N4R-tau-2CA substrates, an effect that was largely impaired in the presence of the enzyme-deficient tau-K18-2CA (FIG. 16c). Taken together, these results are consistent with tau auto-acetylation occurring preferentially in cis (intra-molecularly), but also partly via an in trans mechanism (inter-molecularly).

To evaluate whether tau auto-acetylation can occur in neurons, an in-trans immunofluorescence assay was developed using cultured primary hippocampal neurons. Since mouse tau is not normally acetylated on Lys280 in primary neurons, the question of whether the addition of purified tau-K19 or enzyme-deficient tau-K19-C322A proteins to fixed and permeabilized neurons is sufficient to promote endogenous mouse tau acetylation was examined. Immunofluorescence analysis using anti-Ac-K280, which detects acetylated mouse tau but not the exogenously added 3R-tau enzyme lacking residue Lys280, showed that active tau-K19, but not the enzyme-deficient tau-K19-C322A mutant dramatically increased Ac-K280 immunoreactivity that co-localized with endogenous mouse tau (FIG. 16d). A similar in trans immunofluorescence analysis using transfected HEK293 cells further demonstrated that exogenously added 3R-tau was sufficient to acetylate ectopically expressed 4R-tau present in fixed and permeabilized cells (FIG. 21). Thus, an active tau acetyltransferase is capable of specifically acetylating cellular tau substrates in trans, further supporting a tau auto-acetylation activity.

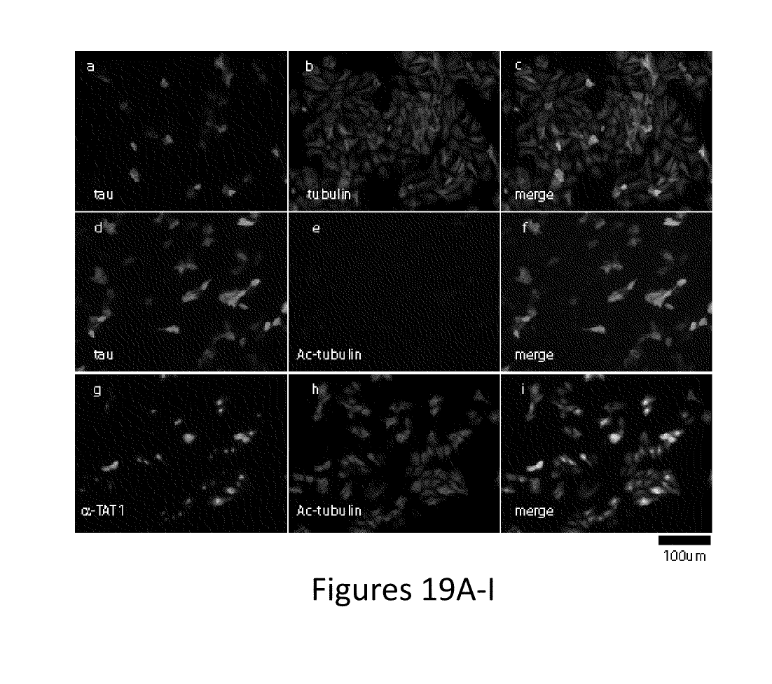

In the present Example, tau has been identified as a bona fide acetyltransferase with sequence and functional similarities to members of the MYST family of lysine acetyltransferases. Using biochemical methods, cell-based assays, and kinetic analysis, evidence is provided herein that tau utilizes essential cysteine residues to catalyze tau auto-acetylation via both intra- and inter-molecular acetylation mechanisms. Several experiments in vitro and in cell-based assays (FIG. 19) precluded a dominant role for tau as a tubulin acetyltransferase. It remains plausible that tau acetylates unknown cytoplasmic or microtubule-associated substrates, the scope of which is slowly emerging with the development of novel proteomic approaches to identify and characterize globally acetylated substrates (Choudhary et al., 2009, Science 325:834-40).

Without wishing to be bound by theory, it is hypothesized that tau itself represents a major target of its own acetyltransferase activity as part of an auto-inhibitory signaling mechanism to prevent tau-MT interactions. Supporting this hypothesis, acetylated tau showed reduced ability to bind and stabilize microtubules in vitro and in cell-based models. Under physiological conditions in normal brain or primary cultured neurons, it has not been possible to detect acetylated tau, suggesting that the majority of tau is normally deacetylated at this residue and bound to MTs. Indeed, it is estimated that tau proteins are ˜99% bound to microtubules in mature neurons (Congdon et al., 2008, J Biol Chem 283:13806-16), and therefore physiological binding to MTs would inhibit tau acetyltransferase activity, likely due to blocked catalytic cysteine residues that actively engage MTs. Tau binding to the cytoskeleton may therefore serve as an off-switch to inhibit tau acetyltransferase activity, which is supported by impaired tau auto-acetylation in the presence of tubulin or microtubules (FIG. 14). In contrast, robust accumulation of acetylated tau aggregates in 4R and 3R-4R-tauopathies is readily observed under distinctly pathological conditions in which tau is detached from microtubules and aggregated to form mature tau lesions. It is further hypothesize that microtubule detachment and activation of tau auto-acetyltransferase activity could represent a pathological event, in which continual self-acetylation gradually shifts the tau-MT binding equilibrium towards cytosolic tau accumulation, providing an increased pool of aggregation-prone tau species. Indeed, tau acetylation has the dual effect of both inhibiting tau-MT interactions and facilitating the formation of β-structure within the repeat regions. The presence of free catalytic cysteine residues as well as a readily available pool of acetyl Co-A would trigger auto-acetylation on lysines within the repeat regions, thereby promoting tau aggregation and NFT formation. Consistent with this model, the levels of acetyl Co-A are elevated in Alzheimer brain (Corrigan et al., 1998, Int J Biochem Cell Biol 30:197-207), supporting increased tau acetyltransferase activity during disease pathogenesis.

The data presented herein indicate that regulatory cross-talk exists between phosphorylation and acetylation in regulating tau activity, a phenomenon that has been similarly demonstrated with several other acetyltransferases including Tip60 and ATF-2 (Kawasaki et al., 2000, Nature 405:195-200; Lemercier et al., 2003, J Biol Chem 278:4713-8). Tau phosphorylation within the microtubule-binding repeats, particularly at Ser262 comprising the 12E8 epitope, has a pronounced inhibitory effect on the affinity of tau for microtubules (Drewes et al., 1995, J Biol Chem 270:7679-88; Mandelkow et al., 1995, Neurobiol Aging 16, 355-62; discussion 362-3). Therefore, the question was asked whether phosphorylation could enhance tau auto-acetylation activity. Indeed, using in vitro or cell-based approaches, individual phospho-mimic mutants (S262E or S2356E) or more prominently, the double mutant (S262E S356E), was sufficient to enhance tau acetyltransferase activity in a cysteine-dependent manner (FIG. 15). These findings suggest that tau phosphorylation within the repeats initiates a conformational change that enhances tau acetyltransferase activity, potentially via increased cofactor binding and catalysis. Supporting this possibility, a phosphorylation-induced conformational change was previously demonstrated using NMR spectroscopy, in which pseudo-phosphorylation on KXGS motifs within the repeats was capable of structurally modifying repeats 1 and 2 resulting in conformational alterations that reduce tau binding to MTs (Fischer et al., 2009, Biochemistry 48:10047-55). Overall, the identification of tau acetyltransferase activity provides a new framework to understand tau pathogenesis, which could provide therapeutic avenues to target tau enzymatic activity in Alzheimer's disease and related tauopathies.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A method of diagnosing a neurodegenerative disorder in a subject, the method comprising determining the level of at least one biomarker in a biological sample obtained from the subject, wherein an elevated level of the biomarker in the sample compared to the level of the biomarker in a control sample is an indication of a neurodegenerative disorder, and further wherein the at least one biomarker comprises acetylated tau.

2. The method of claim 1, wherein the neurodegenerative disorder is at least one disorder selected from the group consisting of Alzheimer's Disease (AD), corticobasal degeneration (CBD), Pick's disease (PiD), progressive supranuclear palsy, argyrophilic grain disease, tangle predominant senile dementia, Guam parkinsonism-dementia complex, frontotemporal dementia, frontotemporal lobar degeneration, FTDP-17, Lytico-Bodig disease, and Parkinson's disease.

3. The method of claim 1, wherein acetylated tau is acetylated on at least one lysine residue selected from the group consisting of K150, K163, K174, K234, K240, K259, K274, K280, K281, K290, K311, K369, and K395.

4. The method of claim 1, wherein the biomarker is detected by a method selected from the group consisting of immunohistochemistry, immunocytochemistry, immunofluorescence, immunoprecipitation, western blotting, and ELISA.

5. A method of diagnosing a neurodegenerative disorder in a subject, the method comprising determining the level of at least one biomarker in a biological sample obtained from the subject, wherein the biomarker differentially discriminates between neurodegenerative disorders, and wherein the biomarker is acetylated tau.

6. The method of claim 5, wherein the biomarker differentially discriminates between neurodegenerative disorders associated with 4R-tau and neurodegenerative disorders not associated with 4R-tau.

7. The method of claim 5, wherein acetylated tau is acetylated on at least one lysine residue selected from the group consisting of K150, K163, K174, K234, K240, K259, K274, K280, K281, K290, K311, K369, and K395.

8. The method of claim 5, wherein the biomarker is detected by at least one method selected from the group consisting of immunohistochemistry, immunofluorescence, western blotting, and ELISA.

9. A composition that specifically recognizes a biomarker associated with a neurodegenerative disorder, wherein the biomarker is acetylated tau.

10. The composition of claim 9, wherein acetylated tau is acetylated on at least one lysine residue selected from the group consisting of K150, K163, K174, K234, K240, K259, K274, K280, K281, K290, K311, K369, and K395.

11. The composition of claim 9, wherein the neurodegenerative disorder Alzheimer's Disease (AD), corticobasal degeneration (CBD), Pick's disease (PiD), progressive supranuclear palsy, argyrophilic grain disease, tangle predominant senile dementia, Guam parkinsonism-dementia complex, frontotemporal dementia, frontotemporal lobar degeneration, FTDP-17, Lytico-Bodig disease, and Parkinson's disease.

12. The composition of claim 9, wherein the composition comprises an antibody or fragment thereof.

13. A composition for treating a neurodegenerative disorder in a subject, wherein the composition reduces the level of tau acetylation).

14. The composition of claim 13, wherein the composition enhances the activity of at least one deacetylase.

15. The composition of claim 13, wherein the composition reduces the activity of at least one acetyltransferase.

16. The composition of claim 13, wherein the composition is selected from the group consisting of a peptide, a nucleic acid, an antibody, and a small molecule.

17. A method of treating a neurodegenerative disorder in a subject, said method comprising administering to the subject an effective amount of a composition, wherein the composition reduces the level of tau acetylation.

18. The method of claim 17, wherein the composition enhances the activity of at least one deacetylase.

19. The method of claim 17, wherein the composition reduces the activity of at least one acetyl-transferase.

20. The method of claim 17, wherein the composition is selected from the group consisting of a peptide, a nucleic acid, an antibody, and a small molecule.


SEQUENCE LISTING (15 sequences)

SEQ ID NO: 1 - RNA, 25 nt, Artificial sequence
  Chemically synthesized
aaaguuggaacucucacggugcagc

SEQ ID NO: 2 - protein, 11 aa, Artificial sequence
  Chemically synthesized
QIINKKLDLSN

SEQ ID NO: 3 - protein, 7 aa, Artificial sequence
  Chemically synthesized
VQIINKK

SEQ ID NO: 4 - protein, 15 aa, Artificial sequence
  Chemically synthesized
GAAPPGQKGQANATR

SEQ ID NO: 5 - protein, 17 aa, Artificial sequence
  Chemically synthesized
IGSLDNITHVPGGGNKK

SEQ ID NO: 6 - protein, 7 aa, Artificial sequence
  Chemically synthesized
TKIATPR

SEQ ID NO: 7 - protein, 10 aa, Artificial sequence
  Chemically synthesized
IPAKTPPAPK

SEQ ID NO: 8 - protein, 10 aa, Artificial sequence
  Chemically synthesized
TPPKSPSSAK

SEQ ID NO: 9 - protein, 12 aa, Artificial sequence
  Chemically synthesized
TPPKSPSSAKSR

SEQ ID NO: 10 - protein, 10 aa, Artificial sequence
  Chemically synthesized
SKIGSTENLK

SEQ ID NO: 11 - protein, 13 aa, Artificial sequence
  Chemically synthesized
HQPGGGKVQIINK

SEQ ID NO: 12 - protein, 10 aa, Artificial sequence
  Chemically synthesized
KLDLSNVQSK

SEQ ID NO: 13 - protein, 19 aa, Artificial sequence
  Chemically synthesized
HVPGGGSVQIVYKPVDLSK

SEQ ID NO: 14 - protein, 21 aa, Artificial sequence
  Chemically synthesized
TDHGAEIVYKSPVVSGDTSPR

SEQ ID NO: 15 - protein, 7 aa, Artificial sequence
  Chemically synthesized
GGNKKIE